(19) United States
(12) Patent Application Publication
(10) Pub. No.: US 2017/0137806 A1
(43) Pub. Date: May 18, 2017

(54) HIGH THROUGHPUT TRANSCRIPTOME ANALYSIS

(71) Applicant: Yeda Research and Development Co. Ltd., Rehovot (IL)

(72) Inventors: Diego JAITIN, Rohovot (IL); Ido Amit, Rehovot (IL); Hadas Keren-Shaul, Rehovot (IL); Liran Valadarsky, Rehovot (IL)

(21) Appl. No.: 15/412,122

(22) Filed: Jan. 23, 2017

Related U.S. Application Data
(63) Continuation of application No. 14/795,039, filed on Jul. 9, 2015.
Application No. 14/795,039 is a continuation-in-part of application No. PCT/IB2014/058153, filed on Jan. 9, 2014.
(60) Provisional application No. 61/750,454, filed on Jan. 9, 2013.

(51) Int. Cl. C12N 15/10; C12Q 1/68

(52) U.S. Cl. CPC C12N 15/1065; C12Q 1/6806

(57) ABSTRACT

Kits and methods for single cell or multiple cell transcriptome analysis are provided. An adapter polynucleotide is disclosed which comprises a double-stranded DNA portion of 15 base pairs and no more than 100 base pairs with a 3′single stranded overhang of at least 3 bases and no more than 10 bases, wherein said double stranded DNA portion is at the 5′ end of the polynucleotide and wherein the sequence of said 3′ single stranded overhang is selected from the group consisting of SEQ ID NOs: 1-8 and 9, wherein the 5′ end of the strand of said double-stranded DNA which is devoid of said 3′ single stranded overhang comprises a free phosphate.

### RELATED APPLICATIONS

This application is a Continuation of U.S. Utility patent application Ser. No. 14/795,039 filed on Jul. 9, 2015.

U.S. Utility patent application Ser. No. 14/795,039 filed on Jul. 9, 2015 is a Continuation-in-Part (CIP) of PCT Patent Application No. PCT/IB2014/058153 having International filing date of Jan. 9, 2014, which claims the benefit of priority under 35 USC §119(e) of U.S. Provisional Patent Application No. 61/750,454 filed on Jan. 9, 2013.

The contents of the above applications are all incorporated by reference as if fully set forth herein in their entirety.

### SEQUENCE LISTING STATEMENT

The ASCII file, entitled 68975SequenceListing.txt, created on Jan. 23, 2017, comprising 25,234 bytes, submitted concurrently with the filing of this application is incorporated herein by reference.

### FIELD AND BACKGROUND OF THE INVENTION

The present invention, in some embodiments thereof, relates to a method of generating cDNA for high throughput transcriptome analysis and kits for same.

Changes in the cell state involve changes in gene expression, such as in the cellular response to extracellular cell division, differentiation or malignant transformation signals. Therefore, obtaining accurate snapshots of the cell transcriptome following a given cell stimulus, or a differentiation state, is essential to understand the cell response in health and disease.

The transcriptome can be profiled by high throughput techniques including SAGE, microarray, and sequencing of clones from cDNA libraries. For more than a decade, oligo-nucleotide microarrays have been the method of choice providing high throughput and affordable costs. However, microarray technology suffers from well-known limitations including insufficient sensitivity for quantifying lower abundant transcripts, narrow dynamic range and biases arising from non-specific hybridizations. Additionally, microarrays are limited to only measuring known/annotated transcripts and often suffer from inaccurate annotations. Sequencing-based methods such as SAGE rely upon cloning and sequencing cDNA fragments.

This approach allows quantification of mRNA abundance by counting the number of times cDNA fragments from a corresponding transcript are represented in a given sample, assuming that cDNA fragments sequenced contain sufficient information to identify a transcript. Sequencing-based approaches have a number of significant technical advantages over hybridization-based microarray methods. The output from sequence-based protocols is digital, rather than analog, obviating the need for complex algorithms for data normalization and summarization while allowing for more precise quantification and greater ease of comparison between results obtained from different samples. Consequently the dynamic range is essentially infinite, if one accumulates enough sequence tags. Sequence-based approaches do not require prior knowledge of the transcriptome and are therefore useful for discovery and annotation of novel transcripts as well as for analysis of poorly annotated genomes. However, until recently the application of sequencing technology in transcriptome profiling has been limited by high cost, by the need to amplify DNA through bacterial cloning, and by the traditional Sanger approach of sequencing by chain termination.

The next-generation sequencing (NGS) technology eliminates some of these barriers, enabling massive parallel sequencing at a high but reasonable cost for small studies. The technology essentially reduces the transcriptome to a series of randomly fragmented segments of a few hundred nucleotides in length. These molecules are amplified by a process that retains spatial clustering of the PCR products, and individual clusters are sequenced in parallel by one of several technologies. Current NGS platforms include the Roche 454 Genome Sequencer, Illumina's Genome Analyzer, and Applied Biosystems' SOLiD. These platforms can analyze tens to hundreds of millions of DNA fragments simultaneously, generate giga-bases of sequence information from a single run, and have revolutionized SAGE and cDNA sequencing technology. For example, the 3′ tag Digital Gene Expression (DGE) uses oligo-dT priming for first strand cDNA synthesis, generates libraries that are enriched in the 3′ untranslated regions of polyadenylated mRNAs, and produces 20-21 base cDNA tags.

Construction of full-length cDNA libraries based on template switching (TS) is known in the art and several high-throughput transcriptome analyses protocols have been devised based on the TS mechanism (Cloonan, N., et al. (2008) Nat. Methods, 5, 613-619; Plessy, C., et al. (2010) Nat. Methods, 7, 528-534; Islam, S., et al., (2011) Genome Res., 21, 1160-1167; Ko, J. H. and Lee, Y. (2006) J. Microbiol. Methods, 64, 297-304; Ramskold, D., et al. (2012), Nat. Biotechnol., 30, 777-782).

U.S. Pat. Nos. 5,962,271 and 5,962,272 teaches methods of generating cDNA polynucleotides based on template switching wherein single stranded oligonucleotides are allowed to hybridize to the cap structure of the 3′-end of a mRNA prior to or concomitant with first-strand cDNA synthesis.

U.S. Patent Application Publication No. 20110189679 teaches methods for generating cDNA from total RNA samples without purification of mRNA. In order to eliminate ribosomal RNA (rRNA) from the sample a tailed primer is used during reverse transcription wherein only a portion thereof hybridizes with the RNA molecules in the total RNA sample.

Additional background art includes Tang et al., Nucleic Acids Research, 2012, 1-12, PCT Publication Nos. WO2013130674 and WO2012148477.

### SUMMARY OF THE INVENTION

According to an aspect of some embodiments of the present invention there is provided an adapter polynucleotide comprising a double-stranded DNA portion with a 3′single stranded overhang, wherein the double stranded DNA portion comprises 15 base pairs and no more than 100 base pairs, wherein said 3′single stranded overhang comprises at least 3 bases and no more than 10 bases, wherein the double stranded DNA portion is at the 5′ end of the polynucleotide and wherein the sequence of the 3′single stranded overhang is selected from the group consisting of SEQ ID NOs: 1-8 and 9, wherein the 5′ end of the strand of the double-stranded DNA which is devoid of the 3′ single stranded overhang comprises a free phosphate.

According to an aspect of some embodiments of the present invention there is provided a library of adapter polynucleotides, wherein each member of the library comprises a double-stranded DNA portion with a 3′ single stranded overhang, wherein the double stranded DNA portion comprises 15 base pairs and no more than 100 base pairs, wherein the a 3′ single stranded overhang comprises at least 3 bases and no more than 10 bases, wherein the 5′ end of the strand of the double-stranded DNA which is devoid of the 3′ single stranded overhang comprises a free phosphate, wherein the double stranded DNA portion is at the 5′ end of the polynucleotide and wherein the sequence of the double stranded portion of the each member of the library is identical, wherein the sequence of the 3′ single stranded overhang of each member of the library is non-identical.

According to an aspect of some embodiments of the present invention there is provided a kit for synthesizing cDNA from an RNA sample comprising the library of adapter polynucleotides described herein and a reverse transcriptase comprising terminal Deoxynucleotidyl Transferase (TdT) activity.

According to an aspect of some embodiments of the present invention there is provided a kit for extending the length of a DNA molecule comprising the library of adapter polynucleotides described herein and a ligase enzyme.

According to an aspect of some embodiments of the present invention there is provided a method of extending the length of a DNA molecule comprising incubating a single stranded DNA molecule with:

(i) an adapter polynucleotide which comprises a double-stranded DNA portion with a 3′ single stranded overhang, wherein the double stranded DNA portion comprises 15 base pairs and no more than 100 base pairs, wherein the 3′ single stranded overhang comprises at least 3 bases and no more than 10 bases, wherein the double stranded DNA portion is at the 5′ end of the polynucleotide, wherein the 5′ end of the strand of the double-stranded DNA which is devoid of the 3′ single stranded overhang comprises a free phosphate and wherein the sequence of the 3′ single stranded overhang is selected such that it is capable of hybridizing to the 3′ end of the single stranded DNA molecule; and

(ii) a ligase enzyme,

under conditions which permit ligation of the adapter polynucleotide to the single stranded DNA molecule, thereby extending the length of a DNA molecule.

According to an aspect of some embodiments of the present invention there is provided a method for generating cDNA, comprising the steps of:

(a) combining an RNA sample with a polydT oligonucleotide under conditions sufficient to allow annealing of the polydT oligonucleotide to mRNA in the RNA sample to produce a polydT-mRNA complex;

(b) incubating the polydT-mRNA complex with a reverse transcriptase comprising terminal Deoxynucleotidyl Transferase (TdT) activity under conditions which permit template-dependent extension of the polydT to generate an mRNA-cDNA hybrid;

(c) contacting the mRNA-cDNA hybrid with Rnase H under conditions which allow generation of a single stranded cDNA molecule; and

(d) incubating the single stranded cDNA molecule with:
(i) an adapter polynucleotide which comprises a double-stranded DNA portion with a 3′ single stranded overhang, wherein the double stranded DNA portion comprises 15 base pairs and no more than 100 base pairs, wherein the 3′ single stranded overhang comprises at least 3 bases and no more than 10 bases, wherein the 5′ end of the strand of the double-stranded DNA which is devoid of the 3′ single stranded overhang comprises a free phosphate, and wherein the sequence of the 3′ single stranded overhang is selected such that it is capable of hybridizing to the 3′ end of the single stranded DNA molecule; and
(ii) a ligase enzyme, under conditions which permit ligation of the adapter polynucleotide to the single stranded cDNA molecule, thereby generating the cDNA.

According to some embodiments of the invention, the double-stranded DNA portion is between 15-30 base pairs.

According to an aspect of some embodiments of the present invention there is provided a kit for transcriptome analysis comprising:

(i) a first oligonucleotide comprising a polyT sequence at its terminal 3′ end, a RNA polymerase promoter sequence at its terminal 5′ end and a barcode sequence positioned between the polyT sequence and the RNA polymerase promoter sequence;

(ii) a second oligonucleotide being a single stranded DNA having a free phosphate at its 5′ end;

(iii) a third oligonucleotide being a single stranded DNA which is fully complementary to the second oligonucleotide.

According to an aspect of some embodiments of the present invention there is provided a method of preparing a cell for transcriptome sequencing comprising:

(a) incubating a plurality of RNA molecules with a reverse transcriptase enzyme and a first oligonucleotide comprising a polyT sequence at its terminal 3′ end, a RNA polymerase promoter sequence at its terminal 5′ end and a barcode sequence positioned between the polyT sequence and the RNA polymerase promoter sequence under conditions that allow synthesis of a single stranded DNA molecule from the RNA;

(b) synthesizing a complementary sequence to the single stranded DNA molecule so as to generate a double stranded DNA molecule;

(c) incubating the double stranded DNA molecule with a T7 RNA polymerase under conditions which allow synthesis of amplified RNA from the double stranded DNA molecule;

(d) fragmenting the amplified RNA into fragmented RNA molecules of about 200 nucleotides;

(e) incubating the fragmented RNA molecules with a ligase enzyme and a second oligonucleotide being a single stranded DNA and having a free phosphate at its 5′end under conditions that allow ligation of the second oligonucleotide to the fragmented RNA molecules so as to generate extended RNA molecules; and

(f) incubating the extended RNA molecules with a third oligonucleotide being a single stranded DNA and which is complementary to the second oligonucleotide, thereby preparing the cell for transcriptome sequencing.

According to an aspect of some embodiments of the present invention there is provided a method of amplifying a plurality of gene sequences in an RNA sample comprising:

(a) contacting the RNA sample with a polydT oligonucleotide and a reverse transcriptase under conditions that allow synthesis of single stranded DNA molecules from the RNA, wherein a 5′ end of the polydT oligonucleotide is coupled to a barcoding sequence which comprises a cell identifier and a unique molecular identifier, and wherein a 5′ end of the barcoding sequence is coupled to a predetermined DNA sequence; and

(b) performing a multiplex PCR reaction on the single stranded DNA molecules using primer pairs which amplify a plurality of sequences of interest, wherein a first primer of each of the primer pairs hybridizes to the single stranded DNA molecules at a position which corresponds to the predetermined DNA sequence and a second primer of each of the primer pairs hybridizes to the single stranded DNA molecules at a position which encodes a target gene of interest, wherein each of the second primers of the primer pairs are coupled to an identical DNA sequence, thereby amplifying the plurality of gene sequences in the RNA sample.

According to some embodiments of the invention, the kit further comprises a T4 RNA ligase and/or a reverse transcriptase.

According to some embodiments of the invention, the first, the second and the third oligonucleotide are each packaged in a separate container.

According to some embodiments of the invention, the second oligonucleotide has a C3 spacer at its 3′end.

According to some embodiments of the invention, the second and the third oligonucleotide are between 10-50 nucleotides in length.

According to some embodiments of the invention, the second and the third oligonucleotide are between 15 and 25 nucleotides in length.

According to some embodiments of the invention, the first oligonucleotide is no longer than 100 nucleotides.

According to some embodiments of the invention, the first oligonucleotide comprises a sequence as set forth in SEQ ID NO: 114.

According to some embodiments of the invention, the method is performed on a plurality of single cells, wherein the barcode sequence indicates the identity of the cell.

According to some embodiments of the invention, the method further comprises pooling the single stranded DNA molecules synthesized in step (a), the pooling being effected prior to step (b).

According to some embodiments of the invention, the 3′ single stranded overhang comprises the sequence as set forth in SEQ ID NO: 1.

According to some embodiments of the invention, the library comprises at least 50 members.

According to some embodiments of the invention, the sequence of the 3′ single stranded overhang of the each member of the library conforms to a representative sequence being selected from the group consisting of SEQ ID NOs: 1-8 and 9.

According to some embodiments of the invention, the sequence of the 3′ single stranded overhang of the each member of the library conforms to a representative sequence being selected from the group consisting of SEQ ID NOs: 1, 3-7 and 9.

According to some embodiments of the invention, the representative sequence is set forth in SEQ ID NO: 1.

According to some embodiments of the invention, the reverse transcriptase comprises Moloney Murine Leukemia Virus Reverse Transcriptase (MMLV-RT).

According to some embodiments of the invention, the kit further comprises at least one of the following components: (i) a ligase; (ii) a polydT oligonucleotide; (iii) a DNA polymerase; (iv) MgCl₂ (v) a PCR primer; and (vi) RNase H.

According to some embodiments of the invention, the 5′ end of the polydT oligonucleotide is coupled to a barcoding sequence.

According to some embodiments of the invention, the polydT oligonucleotide is attached to a solid support.

According to some embodiments of the invention, the 5′ terminus of the polydT oligonucleotide comprises an RNA polymerase promoter sequence.

According to some embodiments of the invention, the 3′ single stranded overhang is selected from the group consisting of SEQ ID NOs: 1-8 and 9.

According to some embodiments of the invention, the single stranded DNA molecule comprises a 3′ terminal CCC nucleic acid sequence.

According to some embodiments of the invention, the single stranded DNA molecule comprises a barcode.

According to some embodiments of the invention, the method further comprises amplifying the cDNA molecule following step (d).

According to some embodiments of the invention, the method further comprises selecting mRNA from the RNA sample prior to step (a).

According to some embodiments of the invention, the 5′ end of the polydT oligonucleotide is coupled to a barcoding sequence.

According to some embodiments of the invention, the polydT oligonucleotide is attached to a solid support.

According to some embodiments of the invention, the RNA sample is derived from a single biological cell.

According to some embodiments of the invention, the RNA sample is derived from a population of biological cells.

According to some embodiments of the invention, the method further comprises amplifying the quantity of RNA in the RNA sample prior to step (a).

According to some embodiments of the invention, the amplifying is effected by:

(a) contacting the RNA with a polydT oligonucleotide having a RNA polymerase promoter sequence at its terminal 5′ end under conditions sufficient to allow annealing of the polydT oligonucleotide to the RNA to produce a polydT-mRNA complex;

(b) incubating the polydT-mRNA complex with a reverse transcriptase devoid of terminal Deoxynucleotidyl Transferase (TdT) activity under conditions which permit template-dependent extension of the polydT to generate an mRNA-cDNA hybrid;

(c) synthesizing a double stranded DNA molecule from the mRNA-cDNA hybrid; and

(d) transcribing RNA from the double stranded DNA molecule.

According to some embodiments of the invention, the method further comprises performing a PCR reaction using a single primer pair which further amplifies the plurality of sequences of interest, wherein a first primer of the single primer pair hybridizes to the predetermined DNA sequence and a second primer of the single primer pair hybridizes to the identical DNA sequence.

According to some embodiments of the invention, the first primer of the single primer pair and the second primer of the single primer pair are coupled to sequencing adaptors.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a TapeStation™ readout of an exemplary cDNA library produced by template switching.

FIG. 2A is a TapeStation™ readout of an exemplary cDNA library produced according to embodiments of the present invention; and

FIG. 2B is a TapeStation™ readout of an exemplary cDNA library produced according to embodiments of the present invention.

FIG. 3A is a graph representing the dynamic range obtained when synthesizing a cDNA library according to embodiments of the present invention.

FIG. 3B is a graph representing the dynamic range obtained when synthesizing a cDNA library according to the Digital Gene Expression (DGE) method.

FIG. 4 is a schematic representation of the method of cDNA synthesis according to embodiments of the present invention.

FIG. 5 is a schematic representation of the method of cDNA synthesis performed on single cells according to embodiments of the present invention.

FIGS. 6A-F. Massively parallel single cell RNA-seq. (A) Schematic diagram of the massively parallel approach to single cell RNA-seq, involving the use of randomized molecular tags to initially label poly-A tailed RNA molecules, followed by pooling labeled samples and performing two rounds of amplification, generating sequencing ready material (see FIG. 10 for an expanded version). (B) The presently described random molecular tagging approach leads to low-penetrance but highly un-biased sampling of 500-5000 RNA molecules per cell. Consequently, the distribution of RNA counts per cell in homogeneous populations behaves remarkably as expected from a distribution of repeated independent sampling from a single cell pool of molecules. Shown here is the distribution of molecule counts per cell for the housekeeping gene Actb, with a mode at 3 and a variation in the range of 1 to 9. (C) The experimental pipeline depicted above generates hundreds to thousands of molecules per cell for up to 1,000 cells in a single sequencing experiment. Homogeneous subpopulations of single cells can then be pooled together to generate accurate estimates of transcriptional states across a large number of genes. Shown here are mean mRNA counts computed for independent sets of 10 to 40 cells, representing 1-4% of a 1000 cell sample. Confidence intervals based on a binomial sampling variance are depicted in red. (D) Cumulative distribution of molecule per cell on 891 CD11c⁺ cells. Median coverage is at 1360 distinct molecules. (E) Shown is the number of genes (Y-axis) covered by a minimum of cells (X-axis), indicating over 4000 genes were sequenced in at least 50 cells. (F) Sampling vs. biological variance in our dataset. The present inventors plot for each gene the variance in mRNA molecule counts normalized by the mean. Also shown are the empirical median (dashed line) and a 1-99% confidence band (gray) for the variance/mean ratio. Data from biologically homogeneous pDC sorted cells (left) represent tight scaling of variance with the mean. In contrast, the CD11c⁺ data (right) is characterized by an overall higher variance and many specific genes with high variance over mean ratios.

FIGS. 7A-F: Functional classification of immune cell types in a CD11c enriched splenic cell population. (A) Clustered cell correlation matrix reveals distinct subclasses of cells with similar transcriptional signatures. The color-coded matrix represents correlations between normalized single cell mRNA counts (for cells with 1500 molecules or more). Groups of strongly correlated cells that are used to initialize a probabilistic mixture model are numbered and marked with white frames. (B) Circular a-posteriori projection (CAP, see methods) summarizing the predictions of the probabilistic mixture model for the CD11c⁺ cells. Each class is positioned on the unit circle with relative spacing that reflects inter-class similarities. Each cell is projected into the two dimensional sphere based on the posterior probability of its association with the different classes. It will be noted that the dimensions of the CAP plot should not be interpreted linearly or as principle components. (C) Mean single cell mRNA counts for a select subset of the genes that strongly mark each of the inferred CD11c⁺ subpopulations. (D) For each CD11c⁺ subpopulation, the correlation of the pooled single cell mRNA count and a set of 34 ImmGen microarray-based gene expression profiles defining different hematopoietic cell types are shown. Bar plots depicting correlations coefficients are shown in gray, and for each subpopulation the most correlated group of cell types is colored specifically as indicated. (E) FACS analysis was used to validate the estimated frequencies of B cells and pDCs in the CD11c⁺ pool. Shown are independent experiments analyzing CD11c vs. CD19 (a B cell marker) and Bst2/PDCA-1 (a pDC marker). B and pDC subpopulation frequencies are shown above the gating frame (gray). (F) Boxplots show the distribution of selected marker genes distinguishing CD11c⁺ subpopulations.

FIGS. 8A-E. FACS sorted populations through the single cell RNA-seq prism. (A) Shown are CAP-plots depicting single cell RNA-seq datasets acquired from four independent sorting experiments enriching for pDC, B cells, NK cells and monocytes. Sorted cells are shown in red, with the background distribution of the CD11c⁺ pool indicated by a background gray scale density map. (B) Clustered cell correlation matrix of FACS sorted pDC cells is shown (upper panel). The pDC population is defined by dozens of specifically expressed genes (right panel), but according to the present analysis there are no significantly correlated subpopulations within it, as indicated by the cell correlation matrix. (C) In contrast, a FACS sorted B cells populations show a clear two-cluster structure in its cell correlation matrix. (D-E) Pooled single cell mRNA counts define the functional states within the two identified B cell subpopulations. ImmGen gene expression profiles for some of the genes discriminating the B cell subpopulations are shown using a black-red color-coding. Many of these discriminating genes are not bona-fide B-cell markers.

FIGS. 9A-E. Three classes of functional states, with different level of heterogeneity define the splenic DC population. (A) The CAP-plot of the CD11c⁺ pool, indicating the three classes associated with typical DC transcriptional state. (B) Clustered cell correlation matrix identifies three broad classes of gene expression within the DC population. (C) Single cell mRNA counts of DC marker genes are shown using color-coded (purple-red) boxes for each cell (X-axis). Cells from all three groups share (albeit variably) common characteristics of DCs. (D) Comparison of FACS and single cell RNA-based sorting was facilitated by FACS sorting and sequencing RNA from three DC subpopulations (CD8^{high} CD86⁺, CD8^{inter} CD86⁻ and CD4⁺ ESAM⁺; gating is shown by gray boxes in the corresponding FACS plots on the right. The FACS sorted single cell profiles were then projected on the DC three-class mixture model, and the fraction of cells mapping to each class was computed. Shown are bar-graphs depicting the estimated fraction of cells in each class for the three FACS populations. (E) Shown are pooled single cell mRNA mean counts (left) side by side with ImmGen gene expression data for three sorted DC classes (right). Genes that were specifically enriched in at least one of the three classes were selected for presentation.

FIG. 10. Schematic diagram presenting the process of converting single cell RNA samples to sequencing ready DNA libraries. Shown are ten experimental steps describing how RNA is tagged, pooled, amplified, fragmented, and how library construction is being performed. Colored lines represent RNA (blue) or DNA (black) molecules, or oligos and primers (see methods for a detailed description).

FIG. 11 is a schematic diagram illustrating cell capture plate preparation.

FIG. 12 is a schematic diagram illustrating RT reaction mix addition.

FIG. 13 is a schematic diagram illustrating Pooling 384-well to two rows in 96 wells.

FIG. 14 is a schematic diagram illustrating a method of sequencing selected RNA molecules according to embodiments of the present invention. Capture plates are prepared with a different RT primer in each well. One cell is sorted per well and, after RT, these cells are pooled into one tube. Multiple gene-specific primers and one common reverse primer are added into this tube for gene enrichment. After cleanup, another PCR is carried out to add sequencing adapters.

FIG. 15 is a scatter illustrating bulk expression levels of different genes in B lymphocytes and natural killer (NK) cells measured according to embodiments of the present invention.

### DESCRIPTION OF SPECIFIC EMBODIMENTS OF THE INVENTION

The present invention, in some embodiments thereof, relates to a method of generating cDNA for high throughput transcriptome analysis and kits for same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The dynamic and functionally diverse nature of cell populations within tissues and organs is a hallmark of multi-cellular organisms. Nevertheless, unbiased and comprehensive classification of tissues into well-defined and functionally coherent cell subpopulations is currently lacking. The present inventors have now developed a new approach to this classical problem based on massively parallel sequencing of RNA from cellular samples. Using a technology that combines randomized RNA labeling and extensive controls the present inventors enable sampling of mRNA molecules from a multitude of cellular samples in a single experiment.

In one embodiment, the present inventors label the 3′ end of cDNA molecules using a single stranded DNA primer. This method has proved to be particularly useful when the quantity of RNA present is small (for example for analysis of single cells). Single cell transcriptional profiles allow for unbiased high-resolution characterization of functional states within cell-type subpopulations.

In another embodiment, the present inventors label the 3′ end of cDNA molecules using a unique double stranded adapter.

This unique double stranded adapter may be used to extend any single stranded cDNA molecule.

The extension process takes advantage of the terminal transferase activity of reverse transcriptase enzymes which exhibits terminal deoxynucleotidyl transferase activity (e.g. Moloney murine leukemia virus (MMLV)). Such enzymes allow for the addition of non-templated nucleotides (predominantly cytidines) once it reaches the 5′ end of the RNA molecule, especially in the presence of manganese. This activity forms an overhang of on average three nucleotides at the 3′ end of the cDNA:RNA hybrid after reverse transcription of the RNA molecule and serves as a useful anchor for the 5′ site.

By using a double stranded adapter with an overhang for ligation matching the terminal transferase activity (for example GGGNNN—SEQ ID NO: 1) and a double stranded DNA ligase, the present inventors have shown that it is possible to ligate the single stranded cDNA to the adaptor in a mock double-stranded DNA ligation reaction. The double-stranded DNA portion of the adapter may encode sequences useful for a variety of functions. For example, the adapter may serve as a target site for primer attachment during a downstream PCR and/or sequencing reaction.

The present inventors further contemplate incorporation of a barcoded sequence at the 5′ end of the cDNA molecule with the aid of a barcoded polydT oligonucleotide during the reverse transcription reaction, allowing for the creation of a streamlined process for building barcoded cDNA libraries for rapid and accurate transcriptome analysis by deep sequencing.

Thus, according to one aspect of the present invention there is provided a method of extending the length of a DNA molecule comprising incubating a single-stranded DNA molecule with:

(i) an adapter polynucleotide which comprises a double-stranded DNA portion of 15 base pairs and no more than 100 base pairs with a 3′single stranded overhang of at least 3 bases and no more than 10 bases, wherein the double stranded DNA portion is at the 5′ end of the polynucleotide and wherein the sequence of the 3′single stranded overhang is selected such that it is capable of hybridizing to the 3′ end of the single stranded DNA molecule; and

(ii) a ligase enzyme,

under conditions which permit ligation of the adapter polynucleotide to the single stranded DNA molecule, thereby extending the length of a DNA molecule.

The single-stranded DNA molecules may be derived from any source including non-cellular sources comprising nucleic acid (e.g., a virus) or from a cell-based organism (e.g., member of archaea, bacteria, or eukarya domains). The single-stranded DNA molecules may be obtained from a subject, e.g., a plant, fungi, eubacteria, archaebacteria, protest, or animal. The subject may be an organism, either a single-celled or multi-cellular organism. The source may be cultured cells, which may be primary cells or cells from an established cell line, among others. The source may be a cellular sample isolated initially from a multi-cellular organism in any suitable form. In some embodiments, the source is an environmental sample, e.g., air, water, agricultural, or soil.

Isolation, extraction or derivation of DNA may be carried out by any suitable method. Isolating DNA from a biological sample generally includes treating a biological sample in such a manner that genomic DNA present in the sample is extracted and made available for analysis. Any isolation method that results in extracted genomic DNA may be used in the practice of the present invention. It will be understood that the particular method used to extract DNA will depend on the nature of the source.

Methods of DNA extraction are well-known in the art. A classical DNA isolation protocol is based on extraction using organic solvents such as a mixture of phenol and chloroform, followed by precipitation with ethanol (J. Sambrook et al., “Molecular Cloning: A Laboratory Manual”, 1989, 2.sup.nd Ed., Cold Spring Harbour Laboratory Press: New York, N.Y.). Other methods include: salting out DNA extraction (P. Sunnucks et al., Genetics, 1996, 144: 747-756; S. M. Aljanabi and I. Martinez, Nucl. Acids Res. 1997, 25: 4692-4693), trimethylammonium bromide salts DNA extraction (S. Gustincich et al., BioTechniques, 1991, 11: 298-302) and guanidinium thiocyanate DNA extraction (J. B. W. Hammond et al., Biochemistry, 1996, 240: 298-300).

There are also numerous versatile kits that can be used to extract DNA from tissues and bodily fluids and that are commercially available from, for example, BD Biosciences Clontech (Palo Alto, Calif.), Epicentre Technologies (Madison, Wis.), Gentra Systems, Inc. (Minneapolis, Minn.), MicroProbe Corp. (Bothell, Wash.), Organon Teknika (Durham, N.C.), and Qiagen Inc. (Valencia, Calif.). User Guides that describe in great detail the protocol to be followed are usually included in all these kits. Sensitivity, processing time and cost may be different from one kit to another. One of ordinary skill in the art can easily select the kit(s) most appropriate for a particular situation.

The sample may be processed before the method is carried out, for example DNA purification may be carried out following the extraction procedure. The DNA in the sample may be cleaved either physically or chemically (e.g. using a suitable enzyme). Processing of the sample may involve one or more of: filtration, distillation, centrifugation, extraction, concentration, dilution, purification, inactivation of interfering components, addition of reagents, and the like.

According to a particular embodiment, the single-stranded DNA molecules are generated by denaturing double stranded DNA molecules. The denaturation step generally comprises heating the double stranded to an elevated temperature and maintaining it at the elevated temperature for a period of time sufficient for any double-stranded nucleic acid present in the reaction mixture to dissociate. For denaturation, the temperature of the reaction mixture is usually raised to, and maintained at, a temperature ranging from about 85° C. to about 100° C., usually from about 90° C. to about 98° C., and more usually from about 93° C. to about 96° C. for a period of time ranging from about 3 to about 120 seconds, usually from about 5 to about 30 seconds.

According to another embodiment, the single-stranded DNA molecules are synthesized in vitro from an RNA sample. Thus, according to one embodiment, the single-stranded DNA molecule is cDNA. The RNA sample may comprise RNA from a population of cells or from a single cell. The RNA may comprise total RNA, mRNA, mitochondrial RNA, chloroplast RNA, DNA-RNA hybrids, viral RNA, cell free RNA, and mixtures thereof.

It will be appreciated that the RNA may be amplified in vitro using methods known in the art and as further described below.

According to a preferred embodiment, the RNA is amplified as described in Example 3, herein below or Example 5 herein below.

Optionally, a polyA tail can be added to the 3′ end of the RNA, e.g., via enzymatic addition of adenosine residues by a polyA polymerase, a terminal transferase, or an RNA ligase.

For synthesis of cDNA, template mRNA may be obtained directly from lysed cells or may be purified from a total RNA sample. The total RNA sample may be subjected to a force to encourage shearing of the RNA molecules such that the average size of each of the RNA molecules is between 100-300 nucleotides, e.g. about 200 nucleotides. To separate the heterogeneous population of mRNA from the majority of the RNA found in the cell, various technologies may be used which are based on the use of oligo(dT) oligonucleotides attached to a solid support. Examples of such oligo(dT) oligonucleotides include: oligo(dT) cellulose/spin columns, oligo(dT)/magnetic beads, and oligo(dT) oligonucleotide coated plates.

Generation of single stranded DNA from RNA requires synthesis of an intermediate RNA-DNA hybrid. For this, a primer is required that hybridizes to the 3′ end of the RNA. Annealing temperature and timing are determined both by the efficiency with which the primer is expected to anneal to a template and the degree of mismatch that is to be tolerated.

The annealing temperature is usually chosen to provide optimal efficiency and specificity, and generally ranges from about 50° C. to about 80° C., usually from about 55° C. to about 70° C., and more usually from about 60° C. to about 68° C. Annealing conditions are generally maintained for a period of time ranging from about 15 seconds to about 30 minutes, usually from about 30 seconds to about 5 minutes.

A “primer,” as used herein, refers to a nucleotide sequence, generally with a free 3′-OH group, that hybridizes with a template sequence (such as one or more target RNAs, or a primer extension product) and is capable of promoting polymerization of a polynucleotide complementary to the template. A “primer” can be, for example, an oligonucleotide. A primer may contain a non-hybridizing sequence that constitutes a tail on the primer. A primer may still be hybridizing even though its sequences are not completely complementary to the target.

The primers of the invention are usually oligonucleotide primers. A primer is generally an oligonucleotide that is employed in an extension by a polymerase along a polynucleotide template such as in, for example, PCR.

The oligonucleotide primer is often a synthetic polynucleotide that is single stranded, containing a sequence at its 3′-end that is capable of hybridizing with a sequence of the target polynucleotide. Normally, the 3′ region of the primer that hybridizes with the target nucleic acid has at least 80%, preferably 90%, more preferably 95%, most preferably 100%, complementarity to a sequence or primer binding site. “Complementary”, as used herein, refers to complementarity to all or only to a portion of a sequence. The number of nucleotides in the hybridizable sequence of a specific oligonucleotide primer should be such that stringency conditions used to hybridize the oligonucleotide primer will prevent excessive random non-specific hybridization. Usually, the number of nucleotides in the hybridizing portion of the oligonucleotide primer will be at least as great as the defined sequence on the target polynucleotide that the oligonucleotide primer hybridizes to, namely, at least 5, at least 6, at least 7, at least 8, at least 9, at least 10, at least 11, at least 12, at least 13, at least 14, at least 15, at least about 20, and generally from about 6 to about 10 or 6 to about 12 of 12 to about 200 nucleotides, usually about 20 to about 50 nucleotides. In general, the target polynucleotide is larger than the oligonucleotide primer or primers as described previously.

According to a specific embodiment, the primer comprises a polydT oligonucleotide sequence.

Preferably the polydT sequence comprises at least 5 nucleotides. According to another is between about 5 to 50 nucleotides, more preferably between about 5-25 nucleotides, and even more preferably between about 12 to 14 nucleotides.

The present invention further contemplates that the primer comprises a barcode sequence (i.e. identification sequence). The barcode sequence is useful during multiplex reactions when a number of samples are pooled in a single reaction. The barcode sequence may be used to identify a particular molecule, sample or library. The barcode sequence is attached to the 5′ end of the polydT oligonucleotide. The barcode sequence may be between 3-400 nucleotides, more preferably between 3-200 and even more preferably between 3-100 nucleotides. Thus, the barcode sequence may be 6 nucleotides, 7 nucleotides, 8, nucleotides, nine nucleotides or ten nucleotides. Examples of barcoding sequences are provided in Table 1 herein below.

TABLE 1

| Barcode name | sequence |
|---|---|
| barcode_1 | TGGTAG (SEQ ID NO: 17) |
| barcode_2 | AGCATG (SEQ ID NO: 18) |
| barcode_3 | ATGTGC (SEQ ID NO: 19) |
| barcode_4 | CGAGCA (SEQ ID NO: 20) |
| barcode_5 | ATTGCT (SEQ ID NO: 21) |
| barcode_6 | GCAACT (SEQ ID NO: 22) |
| barcode_7 | AACTGG (SEQ ID NO: 23) |
| barcode_8 | GCTCAA (SEQ ID NO: 24) |
| barcode_9 | GTTGGT (SEQ ID NO: 25) |
| barcode_10 | TGGACC (SEQ ID NO: 26) |
| barcode_11 | TTATAC (SEQ ID NO: 27) |
| barcode_12 | AGCGAA (SEQ ID NO: 28) |
| barcode_13 | CAAGTT (SEQ ID NO: 29) |
| barcode_14 | GATGTG (SEQ ID NO: 30) |
| barcode_15 | TTCCGA (SEQ ID NO: 31) |
| barcode_16 | ATCCTT (SEQ ID NO: 32) |
| barcode_17 | GTGTGC (SEQ ID NO: 33) |
| barcode_18 | GCCAGA (SEQ ID NO: 34) |
| barcode_19 | GCTATG (SEQ ID NO: 35) |
| barcode_20 | CTCCTG (SEQ ID NO: 36) |
| barcode_21 | CCGACA (SEQ ID NO: 37) |
| barcode_22 | CATAAT (SEQ ID NO: 38) |
| barcode_23 | GGTAGG (SEQ ID NO: 39) |
| barcode_24 | TAAGTA (SEQ ID NO: 40) |
| barcode_25 | TTCTTC (SEQ ID NO: 41) |
| barcode_26 | GATCCT (SEQ ID NO: 42) |
| barcode_27 | ACTGTC (SEQ ID NO: 43) |
| barcode_28 | CATAGG (SEQ ID NO: 44) |
| barcode_29 | AGGCGA (SEQ ID NO: 45) |
| barcode_30 | CGCTAT (SEQ ID NO: 46) |
| barcode_31 | GGCGAC (SEQ ID NO: 47) |
| barcode_32 | TAGAAT (SEQ ID NO: 48) |
| barcode_33 | GTAACG (SEQ ID NO: 49) |
| barcode_34 | TCAGAC (SEQ ID NO: 50) |
| barcode_35 | GCGTAA (SEQ ID NO: 51) |
| barcode_36 | ATTCAA (SEQ ID NO: 52) |
| barcode_37 | TGTCTT (SEQ ID NO: 53) |
| barcode_38 | TTGCTG (SEQ ID NO: 54) |
| barcode_39 | GCTGGA (SEQ ID NO: 55) |
| barcode_40 | CTCTGG (SEQ ID NO: 56) |
| barcode_41 | CAAGGA (SEQ ID NO: 57) |
| barcode_42 | TAACCT (SEQ ID NO: 58) |
| barcode_43 | GATGAC (SEQ ID NO: 59) |
| barcode_44 | CGAAGG (SEQ ID NO: 60) |
| barcode_45 | CCGAGA (SEQ ID NO: 61) |
| barcode_46 | GACAAT (SEQ ID NO: 62) |
| barcode_47 | AGGTTC (SEQ ID NO: 63) |
| barcode_48 | TCATTA (SEQ ID NO: 64) |
| barcode_49 | TGCGTT (SEQ ID NO: 65) |
| vbarcode_50 | GAGTTG (SEQ ID NO: 66) |
| barcode_51 | TTACAG (SEQ ID NO: 67) |
| barcode_52 | TGCTTA (SEQ ID NO: 68) |
| barcode_53 | AACATT (SEQ ID NO: 69) |
| barcode_54 | CCGCTG (SEQ ID NO: 70) |
| barcode_55 | CTGGTC (SEQ ID NO: 71) |
| barcode_56 | TGGATA (SEQ ID NO: 72) |
| barcode_57 | ACCTGT (SEQ ID NO: 73) |
| barcode_58 | TGTTGG (SEQ ID NO: 74) |
| barcode_59 | GACGGC (SEQ ID NO: 75) |
| barcode_60 | CAGATA (SEQ ID NO: 76) |
| barcode_61 | CTTAGT (SEQ ID NO: 77) |
| barcode_62 | AAGGCG (SEQ ID NO: 78) |
| barcode_63 | CTAGGC (SEQ ID NO: 79) |
| barcode_64 | GCAGCA (SEQ ID NO: 80) |
| barcode_65 | TTACCT (SEQ ID NO: 81) |
| barcode_66 | AGTTAG (SEQ ID NO: 82) |
| barcode_67 | TGTTAC (SEQ ID NO: 83) |
| barcode_68 | ATTACA (SEQ ID NO: 84) |
| barcode_69 | GATAAT (SEQ ID NO: 85) |
| barcode_70 | GCATAG (SEQ ID NO: 86) |
| barcode_71 | GTGGAC (SEQ ID NO: 87) |
| barcode_72 | AGACAA (SEQ ID NO: 88) |
| barcode_73 | ATTGTT (SEQ ID NO: 89) |
| barcode_74 | AGGAAT (SEQ ID NO: 90) |
| barcode_75 | TCCTTC (SEQ ID NO: 91) |
| barcode_76 | TAGCGA (SEQ ID NO: 92) |
| barcode_77 | AACTGT (SEQ ID NO: 93) |
| barcode_78 | CTATTG (SEQ ID NO: 94) |
| barcode_79 | ACGGTC (SEQ ID NO: 95) |
| barcode_80 | TGCAGA (SEQ ID NO: 96) |
| barcode_81 | TACAGT (SEQ ID NO: 97) |
| barcode_82 | TGCTGG (SEQ ID NO: 98) |
| barcode_83 | TAGGTC (SEQ ID NO: 99) |
| barcode_84 | CTTGCA (SEQ ID NO: 100) |
| barcode_85 | CATGCT (SEQ ID NO: 101) |
| barcode_86 | ATAGCG (SEQ ID NO: 102) |
| barcode_87 | GATATC (SEQ ID NO: 103) |
| barcode_88 | GTTACA (SEQ ID NO: 104) |
| barcode_89 | CGACCT (SEQ ID NO: 105) |
| barcode_90 | CCGCAG (SEQ ID NO: 106) |
| barcode_91 | GGCTGC (SEQ ID NO: 107) |
| barcode_92 | GATTAA (SEQ ID NO: 108) |
| barcode_93 | GCACCT (SEQ ID NO: 109) |
| barcode_94 | CCACAG (SEQ ID NO: 110) |
| barcode_95 | TGCGGC (SEQ ID NO: 10) |
| barcode_96 | ATATAA (SEQ ID NO: 11) |

The primer used for reverse transcription may comprise a tag at its 5′ end. The tag at the 5′ end of the primer that is annealed to the 3′ end of the RNA or to the polyA tail can optionally include one or more ligand, blocking group, phosphorylated nucleotide, phosphorothioated nucleotide, biotinylated nucleotide, digoxigenin-labeled nucleotide, methylated nucleotide, uracil, sequence capable of forming a hairpin structure, oligonucleotide hybridization site, restriction endonuclease recognition site, promoter sequence, and/or cis regulatory sequence.

The primers may include additional sequences (e.g. at the 5′ flanking the barcode) as described in detail in Example 1 below. These sequences may include nucleotides that are necessary for a sequencing process in a downstream reaction. Exemplary sequences that may be added include for example those set forth in SEQ ID NOs: 111 and 112.

Methods of synthesizing primers (e.g. oligonucleotides) are known in the art and are further described herein below.

Following annealing of a primer (e.g. polydT primer) to the RNA sample, an RNA-DNA hybrid may be synthesized by reverse transcription using an RNA-dependent DNA polymerase. Suitable RNA-dependent DNA polymerases for use in the methods and compositions of the invention include reverse transcriptases (RTs). RTs are well known in the art. Examples of RTs include, but are not limited to, Moloney murine leukemia virus (M-MLV) reverse transcriptase, human immunodeficiency virus (HIV) reverse transcriptase, rous sarcoma virus (RSV) reverse transcriptase, avian myeloblastosis virus (AMV) reverse transcriptase, rous associated virus (RAV) reverse transcriptase, and myeloblastosis associated virus (MAV) reverse transcriptase or other avian sarcoma-leukosis virus (ASLV) reverse transcriptases, and modified RTs derived therefrom. See e.g. U.S. Pat. No. 7,056,716. Many reverse transcriptases, such as those from avian myeloblastosis virus (AMV-RT), and Moloney murine leukemia virus (MMLV-RT) comprise more than one activity (for example, polymerase activity and ribonuclease activity) and can function in the formation of the double stranded cDNA molecules. However, in some instances, it is preferable to employ a RT which lacks or has substantially reduced RNase H activity.

RTs devoid of RNase H activity are known in the art, including those comprising a mutation of the wild type reverse transcriptase where the mutation eliminates the RNase H activity. Examples of RTs having reduced RNase H activity are described in US20100203597. In these cases, the addition of an RNase H from other sources, such as that isolated from E. coli, can be employed for the formation of the single stranded cDNA. Combinations of RTs are also contemplated, including combinations of different non-mutant RTs, combinations of different mutant RTs, and combinations of one or more non-mutant RT with one or more mutant RT.

According to a preferred embodiment, the reverse transcriptase comprises terminal Deoxynucleotidyl Transferase (TdT) activity. Examples of such reverse transcriptases include for example Moloney murine leukemia virus (M-MLV) reverse transcriptase (such as Superscript II from Invitrogen, SMARTScribe from Clontech, M-MuLV RNase H minus from New England Biolabs).

Additional components required in a reverse transcription reaction include dNTPS (dATP, dCTP, dGTP and dTTP) and optionally a reducing agent such as Dithiothreitol (DTT) and MnCl₂.

It will be appreciated that when a reverse transcriptase is used that comprises terminal Deoxynucleotidyl Transferase (TdT) activity, following addition of RNaseH, a single stranded DNA molecule is generated that has non-templated nucleotides (predominantly cytidines) at its 3′ end.

Thus, according to one embodiment, the single stranded DNA which is to be extended according to this aspect of the present invention comprises a CCC sequence at its 3′ end.

According to another embodiment, the single-stranded DNA molecule comprises a polydT sequence (and optionally a barcoding sequence) at its 5′ end.

The present invention contemplates that the single-stranded DNA molecules are typically at least 20 nucleotides long, more preferably at least 50 nucleotides long, more preferably at least 100 nucleotides. According to a particular embodiment, the single-stranded DNA molecules are about 200 nucleotides long. According to a particular embodiment, the single-stranded DNA molecules are about 250 nucleotides long. According to a particular embodiment, the single-stranded DNA molecules are about 300 nucleotides long. According to still another embodiment, the single-stranded DNA molecules are no longer than 500 nucleotides long. According to still another embodiment, the single-stranded DNA molecules are no longer than 1000 nucleotides long.

As mentioned, the method of this aspect of the present invention comprises incubating the ssDNA molecule together with an adapter polynucleotide and a ligase enzyme (e.g. T4 or T3 ligase) under conditions (e.g. temperature, buffer, salt, ionic strength, and pH conditions) that allow ligation of the adapter polynucleotide to the single stranded DNA molecule.

The adapter polynucleotide comprises a double-stranded DNA portion of 15 base pairs and no more than 100 base pairs with a 3′single stranded overhang of at least 1 base and no more than 10 bases, wherein the double stranded DNA portion is at the 5′ end of the polynucleotide and wherein the sequence of the 3′single stranded overhang is selected such that it is capable of hybridizing to the 3′ end of the single stranded DNA molecule.

According to a particular embodiment, the 3′ single stranded overhang comprises a GGG nucleic acid sequence.

The 3′ single stranded overhang may comprise an RNA or DNA sequence.

Exemplary contemplated sequences of the 3′ single stranded overhang of the adapter polynucleotide are set forth below:

(SEQ ID NO: 1)
GGGNNN
(SEQ ID NO: 2)
TTT
(SEQ ID NO: 3)
TTTNNN
(SEQ ID NO: 4)
NGG
(SEQ ID NO: 5)
NGGNNN
(SEQ ID NO: 6)
NTT
(SEQ ID NO: 7)
NTTNNN
(SEQ ID NO: 8)
TGG
(SEQ ID NO: 9)
TGGNNN

wherein N is any one of the four nucleotides.

According to one embodiment, the 3′ single stranded overhang of the adapter polynucleotide comprises a random sequence—e.g. a three nucleotide random sequence, a four nucleotide random sequence, a five nucleotide random sequence, a five nucleotide random sequence or a six nucleotide random sequence.

A random sequence is one that is not designed based on a particular or specific sequence in a sample, but rather is based on a statistical expectation (or an empirical observation) that the random sequence is hybridizable (under a given set of conditions) to one or more single stranded DNA sequences in the sample.

In order for ligation to occur, it will be appreciated that the adapter molecule of the present invention comprises a phosphate group in the 5′ end of the strand to be ligated, and an overhang at the complementary strand of at least one nucleotide to allow pairing to occur prior to ligation. According to one embodiment, the overhang is at least three nucleotides long, matching the T4/T3 ligase footprint requirements. According to a particular embodiment, the overhang comprises at least two consecutive guanine nucleotides. According to another embodiment, the overhang comprises at least three consecutive guanine nucleotides.

Since the precise sequence of the 3′ end of the single stranded DNA molecule may not be known, the present invention contemplates incubating the DNA molecule with a plurality of non-identical adapters, each having a different sequence at its overhanging sequence, so as to increase the chance of hybridization.

Thus, according to another aspect of the present invention there is provided a library of adapter polynucleotides, wherein each member of the library comprises a double-stranded DNA portion of 15 base pairs and no more than 100 base pairs with a 3′ single stranded overhang of at least 1 base and no more than 10 bases, wherein the double stranded DNA portion is at the 5′ end of the polynucleotide and wherein the sequence of the double stranded portion of the each member of the library is identical and the sequence of the 3′ single stranded overhang of each member of the library is non-identical.

As used herein, the term “library” when relating to a “library of adapter polynucleotides” refers to a mixture of adapter polynucleotides wherein at least two members, at least 5 members or at least 10 members of the mixture have a non-identical sequence at the 3′ single stranded overhang.

Thus, according to a particular embodiment, all the members of the library have single stranded overhangs that conform to the representative sequence as set forth in SEQ ID NO: 1.

Thus, according to a particular embodiment, all the members of the library have single stranded overhangs that conform to the representative sequence as set forth in SEQ ID NO: 3.

Thus, according to a particular embodiment, all the members of the library have single stranded overhangs that conform to the representative sequence as set forth in SEQ ID NO: 4.

Thus, according to a particular embodiment, all the members of the library have single stranded overhangs that conform to the representative sequence as set forth in SEQ ID NO: 5.

Thus, according to a particular embodiment, all the members of the library have single stranded overhangs that conform to the representative sequence as set forth in SEQ ID NO: 6.

Thus, according to a particular embodiment, all the members of the library have single stranded overhangs that conform to the representative sequence as set forth in SEQ ID NO: 7.

Thus, according to a particular embodiment, all the members of the library have single stranded overhangs that conform to the representative sequence as set forth in SEQ ID NO: 9.

Thus, according to a particular embodiment, all the members of the library have single stranded overhangs that have a 3 nucleotide random sequence.

Thus, according to a particular embodiment, all the members of the library have single stranded overhangs that have a 6 nucleotide random sequence.

The library may comprise members that conform to a combination of representative sequences. Further, the library may comprise additional members—e.g. those that conform to SEQ ID NOs: 2 and 8.

The library of this aspect of the present invention may comprise about 5 members, 10 members, 15 members, 20 members, 25 members, 30 members, 35 members, 40 members, 45 members, 50 members, 55 members, 60 members, 65 members, 70 members, 75 members, 80 members, 85 members, 90 members, 95 members 100 members or more.

The sequence of the double stranded portion of the adapters typically is selected such that they are capable of aiding in a downstream reaction, such as a PCR reaction and/or a sequencing reaction, as further described herein below. Thus, the sequence of the double stranded portion may be capable of hybridizing to a sequencing device or a particular PCR primer. For example, the present invention contemplates that the double stranded DNA portion of the adapter comprises a sequence such that binds to a sequencing platform (flow cell) via an anchor probe binding site (otherwise referred to as a flow cell binding site) whereby it is amplified in situ on a glass slide, such as in the Illumina Genome Analyzer System based on technology described in WO 98/44151, hereby incorporated by reference.

Polynucleotides of the invention (e.g. primers, oligonucleotides and adapters) may be prepared by any of a variety of methods (see, for example, J. Sambrook et al., “Molecular Cloning: A Laboratory Manual”, 1989, 2.sup.nd Ed., Cold Spring Harbour Laboratory Press: New York, N.Y.; “PCR Protocols: A Guide to Methods and Applications”, 1990, M. A. Innis (Ed.), Academic Press: New York, N.Y.; P. Tijssen “Hybridization with Nucleic Acid Probes—Laboratory Techniques in Biochemistry and Molecular Biology (Parts I and II)”, 1993, Elsevier Science; “PCR Strategies”, 1995, M. A. Innis (Ed.), Academic Press: New York, N.Y.; and “Short Protocols in Molecular Biology”, 2002, F. M. Ausubel (Ed.), 5.sup.th Ed., John Wiley & Sons: Secaucus, N.J.). For example, oligonucleotides may be prepared using any of a variety of chemical techniques well-known in the art, including, for example, chemical synthesis and polymerization based on a template as described, for example, in S. A. Narang et al., Meth. Enzymol. 1979, 68: 90-98; E. L. Brown et al., Meth. Enzymol. 1979, 68: 109-151; E. S. Belousov et al., Nucleic Acids Res. 1997, 25: 3440-3444; D. Guschin et al., Anal. Biochem. 1997, 250: 203-211; M. J. Blommers et al., Biochemistry, 1994, 33: 7886-7896; and K. Frenkel et al., Free Radic. Biol. Med. 1995, 19: 373-380; and U.S. Pat. No. 4,458,066.

For example, oligonucleotides may be prepared using an automated, solid-phase procedure based on the phosphoramidite approach. In such a method, each nucleotide is individually added to the 5′-end of the growing oligonucleotide chain, which is attached at the 3′-end to a solid support. The added nucleotides are in the form of trivalent 3′-phosphoramidites that are protected from polymerization by a dimethoxytrityl (or DMT) group at the 5′-position. After base-induced phosphoramidite coupling, mild oxidation to give a pentavalent phosphotriester intermediate and DMT removal provides a new site for oligonucleotide elongation. The oligonucleotides are then cleaved off the solid support, and the phosphodiester and exocyclic amino groups are deprotected with ammonium hydroxide. These syntheses may be performed on oligo synthesizers such as those commercially available from Perkin Elmer/Applied Biosystems, Inc. (Foster City, Calif.), DuPont (Wilmington, Del.) or Milligen (Bedford, Mass.). Alternatively, oligonucleotides can be custom made and ordered from a variety of commercial sources well-known in the art, including, for example, the Midland Certified Reagent Company (Midland, Tex.), ExpressGen, Inc. (Chicago, Ill.), Operon Technologies, Inc. (Huntsville, Ala.), and many others.

Purification of the oligonucleotides of the invention, where necessary or desirable, may be carried out by any of a variety of methods well-known in the art. Purification of oligonucleotides is typically performed either by native acrylamide gel electrophoresis, by anion-exchange HPLC as described, for example, by J. D. Pearson and F. E. Regnier (J. Chrom., 1983, 255: 137-149) or by reverse phase HPLC (G. D. McFarland and P. N. Borer, Nucleic Acids Res., 1979, 7: 1067-1080).

The sequence of oligonucleotides can be verified using any suitable sequencing method including, but not limited to, chemical degradation (A. M. Maxam and W. Gilbert, Methods of Enzymology, 1980, 65: 499-560), matrix-assisted laser desorption ionization time-of-flight (MALDI-TOF) mass spectrometry (U. Pieles et al., Nucleic Acids Res., 1993, 21: 3191-3196), mass spectrometry following a combination of alkaline phosphatase and exonuclease digestions (H. Wu and H. Aboleneen, Anal. Biochem., 2001, 290: 347-352), and the like.

As mentioned above, modified oligonucleotides may be prepared using any of several means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc), or charged linkages (e.g., phosphorothioates, phosphorodithioates, etc). Oligonucleotides may contain one or more additional covalently linked moieties, such as, for example, proteins (e.g., nucleases, toxins, antibodies, signal peptides, poly-L-lysine, etc), intercalators (e.g., acridine, psoralen, etc), chelators (e.g., metals, radioactive metals, iron, oxidative metals, etc), and alkylators. The oligonucleotide may also be derivatized by formation of a methyl or ethyl phosphotriester or an alkyl phosphoramidate linkage. Furthermore, the oligonucleotide sequences of the present invention may also be modified with a label as detailed herein above.

Once the adapter polynucleotide of the present invention is ligated to the single stranded DNA (i.e. further to extension of the single stranded DNA), amplification reactions may be performed.

As used herein, the term “amplification” refers to a process that increases the representation of a population of specific nucleic acid sequences in a sample by producing multiple (i.e., at least 2) copies of the desired sequences. Methods for nucleic acid amplification are known in the art and include, but are not limited to, polymerase chain reaction (PCR) and ligase chain reaction (LCR). In a typical PCR amplification reaction, a nucleic acid sequence of interest is often amplified at least fifty thousand fold in amount over its amount in the starting sample. A “copy” or “amplicon” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable but not complementary to the template), and/or sequence errors that occur during amplification.

A typical amplification reaction is carried out by contacting a forward and reverse primer (a primer pair) to the adapter-extended DNA described herein together with any additional amplification reaction reagents under conditions which allow amplification of the target sequence.

The terms “forward primer” and “forward amplification primer” are used herein interchangeably, and refer to a primer that hybridizes (or anneals) to the target (template strand).

The terms “reverse primer” and “reverse amplification primer” are used herein interchangeably, and refer to a primer that hybridizes (or anneals) to the complementary target strand. The forward primer hybridizes with the target sequence 5′ with respect to the reverse primer.

The term “amplification conditions”, as used herein, refers to conditions that promote annealing and/or extension of primer sequences. Such conditions are well-known in the art and depend on the amplification method selected. Thus, for example, in a PCR reaction, amplification conditions generally comprise thermal cycling, i.e., cycling of the reaction mixture between two or more temperatures. In isothermal amplification reactions, amplification occurs without thermal cycling although an initial temperature increase may be required to initiate the reaction. Amplification conditions encompass all reaction conditions including, but not limited to, temperature and temperature cycling, buffer, salt, ionic strength, and pH, and the like.

As used herein, the term “amplification reaction reagents”, refers to reagents used in nucleic acid amplification reactions and may include, but are not limited to, buffers, reagents, enzymes having reverse transcriptase and/or polymerase activity or exonuclease activity, enzyme cofactors such as magnesium or manganese, salts, nicotinamide adenine dinuclease (NAD) and deoxynucleoside triphosphates (dNTPs), such as deoxyadenosine triphosphate, deoxyguanosine triphosphate, deoxycytidine triphosphate and thymidine triphosphate. Amplification reaction reagents may readily be selected by one skilled in the art depending on the amplification method used.

According to this aspect of the present invention, the amplifying may be effected using techniques such as polymerase chain reaction (PCR), which includes, but is not limited to Allele-specific PCR, Assembly PCR or Polymerase Cycling Assembly (PCA), Asymmetric PCR, Helicase-dependent amplification, Hot-start PCR, Intersequence-specific PCR (ISSR), Inverse PCR, Ligation-mediated PCR, Methylation-specific PCR (MSP), Miniprimer PCR, Multiplex Ligation-dependent Probe Amplification, Multiplex-PCR, Nested PCR, Overlap-extension PCR, Quantitative PCR (Q-PCR), Reverse Transcription PCR (RT-PCR), Solid Phase PCR: encompasses multiple meanings, including Polony Amplification (where PCR colonies are derived in a gel matrix, for example), Bridge PCR (primers are covalently linked to a solid-support surface), conventional Solid Phase PCR (where Asymmetric PCR is applied in the presence of solid support bearing primer with sequence matching one of the aqueous primers) and Enhanced Solid Phase PCR (where conventional Solid Phase PCR can be improved by employing high Tm and nested solid support primer with optional application of a thermal ‘step’ to favor solid support priming), Thermal asymmetric interlaced PCR (TAIL-PCR), Touchdown PCR (Step-down PCR), PAN-AC and Universal Fast Walking.

The PCR (or polymerase chain reaction) technique is well-known in the art and has been disclosed, for example, in K. B. Mullis and F. A. Faloona, Methods Enzymol., 1987, 155: 350-355 and U.S. Pat. Nos. 4,683,202; 4,683,195; and 4,800,159 (each of which is incorporated herein by reference in its entirety). In its simplest form, PCR is an in vitro method for the enzymatic synthesis of specific DNA sequences, using two oligonucleotide primers that hybridize to opposite strands and flank the region of interest in the target DNA. A plurality of reaction cycles, each cycle comprising: a denaturation step, an annealing step, and a polymerization step, results in the exponential accumulation of a specific DNA fragment (“PCR Protocols: A Guide to Methods and Applications”, M. A. Innis (Ed.), 1990, Academic Press: New York; “PCR Strategies”, M. A. Innis (Ed.), 1995, Academic Press: New York; “Polymerase chain reaction: basic principles and automation in PCR: A Practical Approach”, McPherson et al. (Eds.), 1991, IRL Press: Oxford; R. K. Saiki et al., Nature, 1986, 324: 163-166). The termini of the amplified fragments are defined as the 5′ ends of the primers. Examples of DNA polymerases capable of producing amplification products in PCR reactions include, but are not limited to: E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus (United States Biochemicals), Bacillus stereothermophilus (Bio-Rad), or Thermococcus litoralis (“Vent” polymerase, New England Biolabs).

The duration and temperature of each step of a PCR cycle, as well as the number of cycles, are generally adjusted according to the stringency requirements in effect. Annealing temperature and timing are determined both by the efficiency with which a primer is expected to anneal to a template and the degree of mismatch that is to be tolerated. The ability to optimize the reaction cycle conditions is well within the knowledge of one of ordinary skill in the art. Although the number of reaction cycles may vary depending on the detection analysis being performed, it usually is at least 15, more usually at least 20, and may be as high as 60 or higher. However, in many situations, the number of reaction cycles typically ranges from about 20 to about 40.

The above cycles of denaturation, annealing, and polymerization may be performed using an automated device typically known as a thermal cycler or thermocycler. Thermal cyclers that may be employed are described in U.S. Pat. Nos. 5,612,473; 5,602,756; 5,538,871; and 5,475,610 (each of which is incorporated herein by reference in its entirety). Thermal cyclers are commercially available, for example, from Perkin Elmer-Applied Biosystems (Norwalk, Conn.), BioRad (Hercules, Calif.), Roche Applied Science (Indianapolis, Ind.), and Stratagene (La Jolla, Calif.).

Amplification products obtained using primers of the present invention may be detected using agarose gel electrophoresis and visualization by ethidium bromide staining and exposure to ultraviolet (UV) light or by sequence analysis of the amplification product.

According to one embodiment, the amplification and quantification of the amplification product may be effected in real-time (qRT-PCR).

As mentioned herein above the method of synthesizing cDNA may be performed on an amplified RNA sample. This may be particular relevant when the RNA sample is derived from a single cell.

According to one embodiment, the RNA is amplified using the following steps:

(a) contacting the RNA with a polydT oligonucleotide having a RNA polymerase promoter sequence at its terminal 5′ end under conditions sufficient to allow annealing of the polydT oligonucleotide to the RNA to produce a polydT-mRNA complex;

(b) incubating the polydT-mRNA complex with a reverse transcriptase devoid of terminal Deoxynucleotidyl Transferase (TdT) activity under conditions which permit template-dependent extension of the polydT to generate an mRNA-cDNA hybrid;

(c) synthesizing a double stranded DNA molecule from the mRNA-cDNA hybrid; and

(d) transcribing RNA from the double stranded DNA molecule.

The polydT oligonucleotide of this embodiment may optionally comprise a barcoding sequence and/or an adapter sequence required for sequencing, as described herein above.

RNA polymerase promoter sequences are known in the art and include for example T7 RNA polymerase promoter sequence—e.g. SEQ ID NO: 113 (CGATTGAGGCCGGTAATACGACTCACTATAGGGGC).

Reverse transcriptases devoid of terminal Deoxynucleotidyl Transferase (TdT) activity are also known in the art and include for example AffinityScript from Agilent or Superscript III from Invitrogen.

The polydT oligonucleotide may be attached to a solid support (e.g. beads) so that the cDNA which is synthesized may be purified.

Following synthesis of the second strand of the cDNA, RNA may be synthesized by incubating with a corresponding RNA polymerase.

An important aspect of the invention is that the methods and compositions disclosed herein can be efficiently and cost-effectively utilized for downstream analyses, such as next-generation sequencing or hybridization platforms, with minimal loss of biological material of interest.

According to another embodiment the RNA is amplified and then labeled according to the following protocol. This protocol is particularly suitable for analyzing a plurality of samples. The protocol comprises the following steps:

(a) incubating a plurality of RNA molecules with a reverse transcriptase enzyme and a first oligonucleotide comprising a polydT sequence at its terminal 3′ end, a RNA polymerase promoter sequence at its terminal 5′ end and a barcode sequence positioned between the polydT sequence and the RNA polymerase promoter sequence under conditions that allow synthesis of a single stranded DNA molecule from the RNA;

(b) synthesizing a complementary sequence to the single stranded DNA molecule so as to generate a double stranded DNA molecule;

(c) incubating the double stranded DNA molecule with a T7 RNA polymerase under conditions which allow synthesis of amplified RNA from the double stranded DNA molecule;

(d) fragmenting the amplified RNA into fragmented RNA molecules of about 200 nucleotides;

(e) incubating the fragmented RNA molecules with a ligase enzyme and a second oligonucleotide being a single stranded DNA and having a free phosphate at its 5′end under conditions that allow ligation of the second oligonucleotide to the fragmented RNA molecules so as to generate extended RNA molecules; and

(f) incubating the extended RNA molecules with a third oligonucleotide being a single stranded DNA and which is complementary to the second oligonucleotide, thereby preparing the cell for transcriptome sequencing.

Step (a):

The components of this step have already been described herein above. An exemplary sequence of the first oligonucleotide is set forth in SEQ ID NO: 114. Typically, the first oligonucleotide is no longer than 200 nucleotides, more preferably no longer than about 100 nucleotides. This step essentially involves the synthesis of bar-coded, single stranded DNA from each RNA molecule, such that the source of the molecule (i.e. from what sample it is derived) is now branded on the molecule itself. Once the single stranded DNA molecules are labeled, it is then possible to pool individual samples and carry out the protocol on multiple samples in a single container. Following synthesis of the labeled single stranded DNA (and optional pooling), the sample may optionally be treated with an enzyme to remove excess primers, such as exonuclease I. Other options of purifying the single stranded DNA are also contemplated including for example the use of paramagnetic microparticles.

Step (b):

Second strand synthesis of cDNA may be effected by incubating the sample in the presence of nucleotide triphosphates and a DNA polymerase. Commercial kits are available for this step which include additional enzymes such as RNAse H (to remove the RNA strand) and buffers. This reaction may optionally be performed in the presence of a DNA ligase. Following second strand synthesis, the product may be purified using methods known in the art including for example the use of paramagnetic microparticles.

Step (c):

In vitro transcription is carried out using RNA polymerase. Commercially available kits may be used such as the T7 High Yield RNA polymerase IVT kit (New England Biolabs).

Step (d):

Prior to fragmentation of the amplified RNA, the DNA may be removed using a DNA enzyme. The RNA may be purified as well prior to fragmentation. Fragmentation of the RNA may be carried out as known in the art. Fragmentation kits are commercially available such as the Ambion fragmentation kit.

Step (e):

The amplified RNA is now labeled on its 3′ end. For this a ligase reaction is performed which essentially ligates single stranded DNA to the RNA. The single stranded DNA has a having a free phosphate at its 5′end and optionally a blocking moiety at its 3′end in order to prevent head to tail ligation. Examples of blocking moieties include C3 spacer or a biotin moiety. Typically, the ssDNA is between 10-50 nucleotides in length and more preferably between 15 and 25. An exemplary sequence of the ssDNA is set forth in SEQ ID NO: 115.

Step (f):

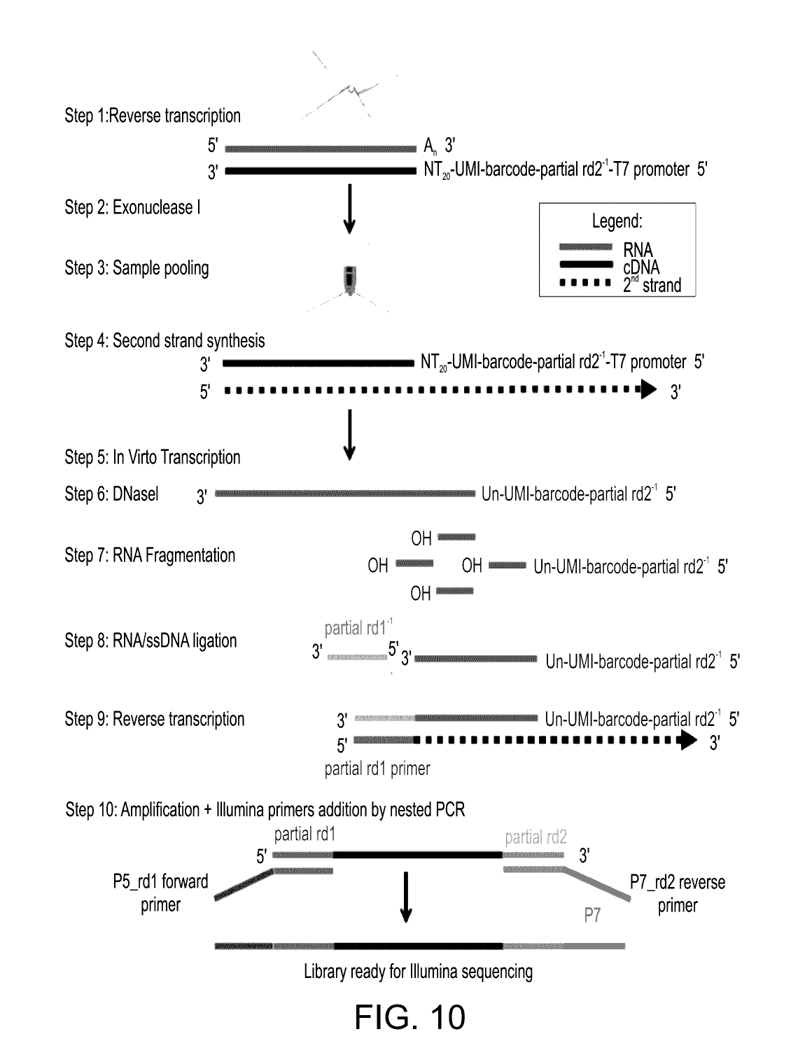

Reverse transcription is then performed using a primer that is complementary to the primer used in the preceding step. An exemplary sequence of this primer is set forth in SEQ ID NO: 116. The library may then be completed and amplified through a nested PCR reaction as illustrated in FIG. 10.

The methods of the invention are useful, for example, for efficient sequencing of a polynucleotide sequence of interest. Specifically the methods of the invention are useful for massively parallel sequencing of a product comprising a plurality of DNA polynucleotides, each having its own barcode as described herein above.

In one embodiment, the invention provides for a method for whole transcriptome sequencing.

Known methods for sequencing include, for example, those described in: Sanger, F. et al., Proc. Natl. Acad. Sci. U.S.A. 75, 5463-5467 (1977); Maxam, A. M. & Gilbert, W. Proc Natl Acad Sci USA 74, 560-564 (1977); Ronaghi, M. et al., Science 281, 363, 365 (1998); Lysov, 1. et al., Dokl Akad Nauk SSSR 303, 1508-1511 (1988); Bains W. & Smith G. C. J. Theor Biol 135, 303-307 (1988); Drnanac, R. et al., Genomics 4, 114-128 (1989); Khrapko, K. R. et al., FEBS Lett 256.118-122 (1989); Pevzner P. A. J Biomol Struct Dyn 7, 63-73 (1989); and Southern, E. M. et al., Genomics 13, 1008-1017 (1992). Pyrophosphate-based sequencing reaction as described, e.g., in U.S. Pat. Nos. 6,274,320, 6,258,568 and 6,210,891, may also be used. In some cases, the methods above require that the nucleic acid attached to the solid surface be single stranded. In such cases, the unbound strand may be melted away using any number of commonly known methods such as addition of NaOH, application of low ionic (e.g., salt) strength solution, enzymatic degradation or displacement of the second strand, or heat processing. Where the solid surface comprises a plurality of beads, following this strand removal step, the beads can be pelleted and the supernatant discarded. The beads can then be resuspended in a buffer, and a sequencing primer or other non-amplification primer can be added. The primer is annealed to the single stranded amplification product. This can be accomplished by using an appropriate annealing buffer and temperature conditions, e.g., as according to standard procedures in the art.

The methods of the invention are useful, for example, for sequencing of an RNA sequence of interest. The sequencing process can be carried out by processing and amplifying a target RNA containing the sequence of interest by any of the methods described herein. Addition of nucleotides during primer extension can be analyzed using methods known in the art, for example, incorporation of a terminator nucleotide, sequencing by synthesis (e.g. pyrosequencing), or sequencing by ligation.

In embodiments wherein the end product is in the form of DNA primer extension products, in addition to the nucleotides, such as natural deoxyribonucleotide triphosphates (dNTPs), that are used in the amplification methods, appropriate nucleotide triphosphate analogs, which may be labeled or unlabeled, that upon incorporation into a primer extension product effect termination of primer extension, may be added to the reaction mixture. Preferably, the dNTP analogs are added after a sufficient amount of reaction time has elapsed since the initiation of the amplification reaction such that a desired amount of second primer extension product or fragment extension product has been generated. Said amount of the time can be determined empirically by one skilled in the art.

Suitable dNTP analogs include those commonly used in other sequencing methods and are well known in the art. Examples of dNTP analogs include dideoxyribonucleotides. Examples of rNTP analogs (such as RNA polymerase terminators) include 3′-dNTP. Sasaki et al., Biochemistry (1998) 95:3455-3460. These analogs may be labeled, for example, with fluorochromes or radioisotopes. The labels may also be labels which are suitable for mass spectroscopy. The label may also be a small molecule which is a member of a specific binding pair, and can be detected following binding of the other member of the specific binding pair, such as biotin and streptavidin, respectively, with the last member of the binding pair conjugated to an enzyme that catalyzes the generation of a detectable signal that could be detected by methods such as colorimetry, fluorometry or chemiluminescence. All of the above examples are well known in the art. These are incorporated into the primer extension product or RNA transcripts by the polymerase and serve to stop further extension along a template sequence. The resulting truncated polymerization products are labeled. The accumulated truncated products vary in length, according to the site of incorporation of each of the analogs, which represent the various sequence locations of a complementary nucleotide on the template sequence.

Analysis of the reaction products for elucidation of sequence information can be carried out using any of various methods known in the art. Such methods include gel electrophoresis and detection of the labeled bands using appropriate scanner, sequencing gel electrophoresis and detection of the radiolabeled band directly by phosphorescence, capillary electrophoresis adapted with a detector specific for the labels used in the reaction, and the like. The label can also be a ligand for a binding protein which is used for detection of the label in combination with an enzyme conjugated to the binding protein, such as biotin-labeled chain terminator and streptavidin conjugated to an enzyme. The label is detected by the enzymatic activity of the enzyme, which generates a detectable signal. As with other sequencing methods known in the art, the sequencing reactions for the various nucleotide types (A, C, G, T or U) are carried out either in a single reaction vessel, or in separate reaction vessels (each representing one of the various nucleotide types). The choice of method to be used is dependent on practical considerations readily apparent to one skilled in the art, such as the nucleotide tri phosphate analogs and/or label used. Thus, for example, when each of the analogs is differentially labeled, the sequencing reaction can be carried out in a single vessel. The considerations for choice of reagent and reaction conditions for optimal performance of sequencing analysis according to the methods of the invention are similar to those for other previously described sequencing methods. The reagent and reaction conditions should be as described above for the nucleic acid amplification methods of the invention.

Other examples of template dependent sequencing methods include sequence by synthesis processes, where individual nucleotides are identified iteratively, as they are added to the growing primer extension product.

Pyrosequencing is an example of a sequence by synthesis process that identifies the incorporation of a nucleotide by assaying the resulting synthesis mixture for the presence of by-products of the sequencing reaction, namely pyrophosphate. In particular, a primer/template/polymerase complex is contacted with a single type of nucleotide. If that nucleotide is incorporated, the polymerization reaction cleaves the nucleoside triphosphate between the alpha and beta phosphates of the triphosphate chain, releasing pyrophosphate. The presence of released pyrophosphate is then identified using a chemiluminescent enzyme reporter system that converts the pyrophosphate, with AMP, into ATP, then measures ATP using a luciferase enzyme to produce measurable light signals. Where light is detected, the base is incorporated, where no light is detected, the base is not incorporated. Following appropriate washing steps, the various bases are cyclically contacted with the complex to sequentially identify subsequent bases in the template sequence. See, e.g., U.S. Pat. No. 6,210,891, incorporated herein by reference in its entirety for all purposes).

In related processes, the primer/template/polymerase complex is immobilized upon a substrate and the complex is contacted with labeled nucleotides. The immobilization of the complex may be through the primer sequence, the template sequence and/or the polymerase enzyme, and may be covalent or noncovalent. In general, preferred aspects, particularly in accordance with the invention provide for immobilization of the complex via a linkage between the polymerase or the primer and the substrate surface. A variety of types of linkages are useful for this attachment, including, e.g., provision of biotinylated surface components, using e.g., biotin-PEG-silane linkage chemistries, followed by biotinylation of the molecule to be immobilized, and subsequent linkage through, e.g., a streptavidin bridge. Other synthetic coupling chemistries, as well as non-specific protein adsorption can also be employed for immobilization. In alternate configurations, the nucleotides are provided with and without removable terminator groups. Upon incorporation, the label is coupled with the complex and is thus detectable. In the case of terminator bearing nucleotides, all four different nucleotides, bearing individually identifiable labels, are contacted with the complex. Incorporation of the labeled nucleotide arrests extension, by virtue of the presence of the terminator, and adds the label to the complex. The label and terminator are then removed from the incorporated nucleotide, and following appropriate washing steps, the process is repeated. In the case of non-terminated nucleotides, a single type of labeled nucleotide is added to the complex to determine whether it will be incorporated, as with pyrosequencing. Following removal of the label group on the nucleotide and appropriate washing steps, the various different nucleotides are cycled through the reaction mixture in the same process. See, e.g., U.S. Pat. No. 6,833,246, incorporated herein by reference in its entirety for all purposes). For example, the Illumina Genome Analyzer System is based on technology described in WO 98/44151, hereby incorporated by reference, wherein DNA molecules are bound to a sequencing platform (flow cell) via an anchor probe binding site (otherwise referred to as a flow cell binding site) and amplified in situ on a glass slide. The DNA molecules are then annealed to a sequencing primer and sequenced in parallel base-by-base using a reversible terminator approach. Typically, the Illumina Genome Analyzer System utilizes flow-cells with 8 channels, generating sequencing reads of 18 to 36 bases in length, generating >1.3 Gbp of high quality data per run.

In yet a further sequence by synthesis process, the incorporation of differently labeled nucleotides is observed in real time as template dependent synthesis is carried out. In particular, an individual immobilized primer/template/polymerase complex is observed as fluorescently labeled nucleotides are incorporated, permitting real time identification of each added base as it is added. In this process, label groups are attached to a portion of the nucleotide that is cleaved during incorporation. For example, by attaching the label group to a portion of the phosphate chain removed during incorporation, i.e., a .beta., .gamma., or other terminal phosphate group on a nucleoside polyphosphate, the label is not incorporated into the nascent strand, and instead, natural DNA is produced. Observation of individual molecules typically involves the optical confinement of the complex within a very small illumination volume. By optically confining the complex, one creates a monitored region in which randomly diffusing nucleotides are present for a very short period of time, while incorporated nucleotides are retained within the observation volume for longer as they are being incorporated. These results in a characteristic signal associated with the incorporation event, which is also characterized by a signal profile that is characteristic of the base being added. In related aspects, interacting label components, such as fluorescent resonant energy transfer (FRET) dye pairs, are provided upon the polymerase or other portion of the complex and the incorporating nucleotide, such that the incorporation event puts the labeling components in interactive proximity, and a characteristic signal results, that is again, also characteristic of the base being incorporated (See, e.g., U.S. Pat. Nos. 6,056,661, 6,917,726, 7,033,764, 7,052,847, 7,056,676, 7,170,050, 7,361,466, 7,416,844 and Published U.S. Patent Application No. 2007-0134128, the full disclosures of which are hereby incorporated herein by reference in their entirety for all purposes). In some embodiments, the nucleic acids in the sample can be sequenced by ligation. This method uses a DNA ligase enzyme to identify the target sequence, for example, as used in the polony method and in the SOLiD technology (Applied Biosystems, now Invitrogen). In general, a pool of all possible oligonucleotides of a fixed length is provided, labeled according to the sequenced position. Oligonucleotides are annealed and ligated; the preferential ligation by DNA ligase for matching sequences results in a signal corresponding to the complementary sequence at that position.

Kits

Any of the compositions described herein may be comprised in a kit. In a non-limiting example the kit comprises the following components, each component being in a suitable container: one or more adapter polynucleotides, a reverse transcriptase comprising terminal Deoxynucleotidyl Transferase (TdT) activity and optionally reagents for additional reactions such as: (i) a ligase; (ii) a polydT oligonucleotide; (iii) a DNA polymerase; (iv) MgCl₂ (v) a PCR primer; and/or (vi) RNAse H.

As mentioned, herein above the polydT oligonucleotide may also comprise a barcoding sequence and additional sequences which aid in downstream sequencing reactions.

In another non-limiting example the kit comprises the following components, each component being in a suitable container: one or more adapter polynucleotide, a ligase enzyme and optionally reagents for additional reactions such as: (i) a reverse transcriptase comprising terminal Deoxynucleotidyl Transferase (TdT) activity; (ii) a polydT oligonucleotide; (iii) a DNA polymerase; (iv) MgCl₂ (v) a PCR primer; and/or (vi) RNAse H.

An exemplary kit for barcoding small amounts of RNA (for example single cells) may comprise at least:

(i) a first oligonucleotide comprising a polydT sequence at its terminal 3′ end, a RNA polymerase promoter sequence at its terminal 5′ end and a barcode sequence positioned between the polydT sequence and the RNA polymerase promoter sequence; and

(ii) a second oligonucleotide being a single stranded DNA having a free phosphate at its 5′ end;

Preferably, the kit also comprises a third oligonucleotide being a single stranded DNA which is fully complementary to the second oligonucleotide.

Each of these components has been described herein above.

Preferably, each of these components are packaged in separate packaging.

Such a kit may comprise additional components such as T4 RNA ligase, RNAseH, DNase and/or a reverse transcriptase.

The containers of the kits will generally include at least one vial, test tube, flask, bottle, syringe or other containers, into which a component may be placed, and preferably, suitably aliquoted. Where there is more than one component in the kit, the kit also will generally contain a second, third or other additional container into which the additional components may be separately placed. However, various combinations of components may be comprised in a container.

When the components of the kit are provided in one or more liquid solutions, the liquid solution can be an aqueous solution. However, the components of the kit may be provided as dried powder(s). When reagents and/or components are provided as a dry powder, the powder can be reconstituted by the addition of a suitable solvent.

A kit will preferably include instructions for employing, the kit components as well the use of any other reagent not included in the kit. Instructions may include variations that can be implemented.

The present inventors have further developed a novel highly accurate targeted single-cell RNA-seq protocol. This protocol amplifies a user-defined set of transcripts from tens of thousands of individual cells in a simple, accurate and rapid protocol. This method of amplification enables exact counting of the number of transcripts of each gene in each cell, giving high-accuracy, low cost and high throughput measurements.

Thus, according to another aspect of the present invention there is provided a method of amplifying a plurality of gene sequences in an RNA sample comprising:

(a) contacting the RNA sample with a polydT oligonucleotide and a reverse transcriptase under conditions that allow synthesis of single stranded DNA molecules from the RNA, wherein a 5′ end of the polydT oligonucleotide is coupled to a barcoding sequence which comprises a cell identifier and a unique molecular identifier, and wherein a 5′ end of the barcoding sequence is coupled to a predetermined DNA sequence; and

(b) performing a multiplex PCR reaction on the single stranded DNA molecules using primer pairs which amplify a plurality of sequences of interest, wherein a first primer of each of the primer pairs hybridizes to the single stranded DNA molecules at a position which corresponds to the predetermined DNA sequence and a second primer of each of the primer pairs hybridizes to the single stranded DNA molecules at a position which encodes a target gene of interest, wherein each of the second primers of the primer pairs are coupled to an identical DNA sequence, thereby amplifying the plurality of gene sequences in the RNA sample.

The RNA sample of this aspect of the present invention may be derived from a plurality of non-homologous cells. According to another embodiment, the RNA sample of this aspect of the present invention is derived from a plurality of homologous cells. According to still another embodiment, the RNA sample of this aspect of the present invention is derived from a single cell. Cell sorting may be effected by FACS or other methods known in the art.

According to a particular embodiment, droplet based microfluidics is used to separate single cells into droplets—see for example WO 2013134261, the contents of which are incorporated herein by reference.

Step (a) of this aspect of the present invention relates to a reverse transcription reaction, in which the reverse transcriptase primer (oligonucleotide) is made up of three components. The first component is a polydT oligonucleotide, the 5′ end of which is coupled to the second component—the barcoding sequence which comprises a cell identifier and a unique molecular identifier. The 5′ end of the second component is coupled to a predetermined DNA sequence. Barcoding sequences and unique molecular identifiers are described herein above. Typically, the unique molecular identifier comprises between 4-20 bases of a known sequence. The predetermined DNA sequence may be of any length—for example between 4-100 bases. According to a particular embodiment, the predetermined DNA sequence encodes an RNA polymerase (e.g. T7) promoter sequence. Other necessary components for reverse transcription include reverse transcriptase enzymes, dNTPs, a reducing agent such as Dithiothreitol (DTT) and MnC12, all of which have been described in detail herein above.

Once cDNA is generated, the cDNA may be pooled from cDNA generated from other cell populations (using the same method as described herein above).

Following synthesis of the labeled single stranded DNA (and optional pooling), the sample may optionally be treated with an enzyme to remove excess primers, such as exonuclease I. Other options of purifying the single stranded DNA are also contemplated including for example the use of paramagnetic microparticles.

The next step, step (b), is to perform a multiplex PCR reaction using at least two primer pairs. According to one embodiment, 2-50 primer pairs are used in a single reaction, 2-40 primer pairs are used in a single reaction, 2-30 primer pairs are used in a single reaction, or 2-25 primer pairs are used in a single reaction. Each primer pair is capable of amplifying a particular gene. It will be appreciated that since each cDNA molecule is transcribed with a predetermined sequence, one of each of the primer pairs is a primer that hybridizes to the predetermined sequence. The second of each of the primer pairs is specific to the target gene which is being amplified.

As used herein, the term “multiplex PCR” refers to the use of polymerase chain reaction to amplify several different DNA targets (genes) simultaneously (as if performing many separate PCR reactions all together in one reaction. PCR has been described in detail herein above.

Selection of primer pairs in the multiplex PCR reaction is within the capability of one of skill in the art. Care should be taken to ensure that the primers generate amplicons of similar lengths.

According to a particular embodiment, the primer which hybridizes to the gene of interest is attached (e.g. coupled) to a second predetermined sequence. This second predetermined sequence is identical for each of the primers. Thus, when performing a second round of amplification, a single amplification reaction may be performed using a first primer that hybridizes to the first predetermined sequence, and a second primer that hybridizes to the second (identical) predetermined sequence. The second round of amplification may also introduce other sequences necessary for sequencing (e.g. sequencing adaptors, such as Illumina adaptors).

Examples of genes of interest that may be amplified include, but are not limited to oncogenes, tumour suppressor genes, inflammatory response genes (e.g. TNF and IL1B) and “stemness” genes.

The components necessary to carry out the method described herein may be provided individually or may be comprised in a kit. Thus, for example, the present inventors contemplate a kit which comprises the reverse transcription primer, as described herein above and primers to carry out the first amplification reaction, and optionally primers to carry out the second amplification reaction. Alternatively, the kit may comprise the reverse transcription primer described herein above and a reverse transcriptase enzyme. Optionally, the kit may also comprise at least one primer necessary to carry out the next step of amplification (for example the primer that hybridizes to the predetermined sequence on the RT primer).

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Protocol for Generating Labeled cDNA from RNA Sample

Reagents:

All reagents must be nuclease-free. Unless indicated otherwise, all reagents should be stored at room temperature (20-25° C.).
Starting material: ˜100 ng of RNA sample for TRANS-seq or one cell (10-30 pg range) for the scTRANSeq protocol.
Dulbecco's phosphate buffered saline (PBS) without Ca²⁺, Mg²⁺, (Beit Haemek Biological Industries, cat.no. 02-023-1).
Water, molecular biology grade (Sigma, cat.no. W4502).
Tris buffer 1 M, pH 8.0, molecular biology grade (Calbiochem, cat.no. 648314).
TRITON X-100, molecular biology grade (Calbiochem, cat.no. 648466).
Lithium Chloride 8 M, molecular biology grade (Sigma, cat.no. L7026).
Tween 20, molecular biology grade (Calbiochem, cat.no. 655204).
RNA Fragmentation buffer (New England Biolabs). Store at −20° C.
Dynabeads oligodT kit (Invitrogen). Store at 4° C.
SMARTScribe reverse transcriptase (Clontech). Store at −20° C.
RNase I, DNase free 500 μg/ml, (New England Biolabs Store at −20° C.
Quick ligase (New England BioLabs, cat.no. M2200—part of the Quick Ligation Kit). Store at −20° C.
2× quick ligation buffer (New England BioLabs, cat.no. M2200—part of the Quick Ligation Kit). Store at −20° C.
Kapa HiFi PCR ready-mix (Kapa) Store at −20° C.
Quant iT 500 ds HS DNA kit (Invitrogen, cat.no. Q32854).
Tapestation (Agilent).
Agencourt AMPure XP (SPRI beads) (Beckman Coulter, cat.no. A63881).

Store at 4° C.
PEG-8,000 (Sigma, cat.no. P5413).
Ethanol 100%.
T4 DNA polymerase 3 u/μl (New England BioLabs, cat.no. M0203). Store at −20° C.
10×T4 ligase buffer (New England BioLabs, cat.no. B0202). Store at −20° C.
dNTP solution set (100 mM; 25 mM each) (New England BioLabs, cat.no. N0446). Aliquot and store at −20° C.
Illumina compatible 96 barcoded adaptors. Store at −20° C.
Indexed RT primer: (NNNNNNNN=barcode for multiplexing):

(SEQ ID NO: 12)
CAAGCAGAAGACGGCATACGAGATNNNNNNNNGTGACTGGAGTTC
AGACGTGTGCTCTTCCGATCTTTTTTTTTTTTTTTTTTTTN

Sense strand: /5SpC3/CTACACGACGCTCTTCCGATCTGGGNNN (SEQ ID NO: 13)

Antisense strand: /5Phos/AGATCGGAAGAGCGTCGTGTAG (SEQ ID NO: 14)

TLA is a double stranded oligo with a 3′ overhang. The sense strand contains a 5′ C3 cap * and a 3′ GGGNNN (SEQ ID NO: 1) overhang; the antisense contains a 5′ phosphate necessary for ligation to take place. Prepare 25 μM by combining 15 μl of 100 μM sense and 100 μM antisense oligos with 30 μl of NEB2×2 (make a 1:5 dilution from 10× stock). Run an annealing program in the PCR cycler: 95° C. 2 min.; decreasing temp from 95° C. down to 20° C. at a 1° C./2 min rate; 20° C. 2 min.; 4° C. forever (total time—2h 40 min). Prepare 1:5 for the ligation reaction.
* a C3 Spacer phosphoramidite reduces the sporadic incidence of adapter dimer formation during ligation from some 0.4% down to less about 0.05%.
Forward+reverse Amplification primers. Store at −20° C.

Forward
(SEQ ID NO: 15)
AATGATACGGCGACCACCGAGATCTACACTCTTTCCCTACACGACGCTCTT
CCGATCT
Reverse
(SEQ ID NO: 16)
CAAGCAGAAGACGGCATACGAGAT.

### Equipment:

DynaMag-2 magnet (Invitrogen, cat.no. 123-21D).
DynaMag-96 magnet (Invitrogen, cat.no. 120-27).
HulaMixer Sample Mixer (Invitrogen, cat.no. 159-20D).
Twin.Tec PCR Plate 96, skirted (Eppendorf, cat.no 0030128648).
Vacuboy Multichannel Vacuum Aspirator (Integra Biosciences, cat.no. 155500).
6 mL Disposable Reservoir Inserts (Labcyte, cat.no. ALL031-01).
3×6 mL Disposable Reservoir holder (Labcyte, cat.no. ALL032-01).
Thermal cycler (Eppendorf MasterCycler Pro, cat.no. 950040015).
Adhesive PCR film (ABgene, cat.no. AB-0558).
Filter tips 200-1200 μl (Rainin, cat.no. RT-L1200F).
Filter tips 20-200 μl (Rainin, cat.no. RT-L200F).
Filter tips 1-20 μl (Rainin, cat.no. RT-L10F).
Multichannel pipette 2-20 μl (PipetLiteXLS LTS, Rainin, cat.no. L12-20XLS).
Multichannel pipette 20-200 μl (PipetLiteXLS LTS, Rainin, cat.no. L12-200XLS).

### Protocol

### RNA Heat Fragmentation—15 Min

1 Preheat the thermal cycler at 94° C.
2 Add 2.5 μl RNA fragmentation buffer to 22.5 μl input RNA in an Eppendorf 96-well PCR plate.
3 Seal the plate with an adhesive PCR film and spindown the plate.
4 Incubate at 94° C. for exactly 5 minutes.
5 Transfer the plate immediately to a −20° C. block to cool down for 2 min.
6 Spindown the plate.
   Polyadenylated (polyA+) mRNA Selection—30 Min
7 During the 5 minutes fragmentation (Step 4) take 12.5 μl oligoT Dynabeads per sample plus some extra (to provide void volume for the multichannel pipettor reservoir) into a 1.7 mL Eppendorf tube.
8 Place the tube on the DynaMag-2, wait a few seconds until the liquid gets clear and the beads form a clear brown pellet at the tube wall and remove the storage buffer.
9 Wash—Remove the tube from the magnet and resuspend the beads in 1.5× volumes of lysis/binding buffer.
10 Repeat step 8 to remove the wash.
11 Remove the tube from the magnet and resuspend in 25 μl lysis/binding buffer per sample (2× the volume of beads taken in step 7) and move to a multichannel reservoir just before use.
12 Add 25 μl washed beads per sample (1:1 by volume) with a multichannel pipettor.
13 Mix 15 times by pipetting up and down and incubate 5 min on the bench.
14 Resuspend by pipetting and incubate another 5 min.
15 Place on the DynaMag-96 magnet, wait a few seconds and remove the supernatant.
16 Wash—Remove plate from magnet and add 100 μl wash buffer; mix thoroughly and place back on the magnet; wait a few seconds until clear and remove the liquid.
17 Repeat step 16 (second wash).

### To Elute the Samples:

18 Add 9.5 μl Tris buffer pH7.5 per well and resuspend the beads by pipetting up and down 15×.
19 Run the thermal cycler at 85° C. (set in advance) and incubate the samples for 2 min at 85° C.
20 Move samples immediately to a hot magnet (several minutes earlier, place a DynaMag-96 plate on a block set at 65° C.) and take 7.6 μl into a clean 96-well PCR plate sitting on ice or a −20° C. block (to avoid sample evaporation).
   Generation of Barcoded cDNA—1.5 Hours
21 Allow barcoded RT primer plate to thaw on the bench.
22 Record the barcode that will be used for each sample.
23 Add 4 μl of corresponding barcoded polydT(20)N RT primer to each well.
24 Mix well, seal the plate and spin down.
25 Heat 3 min at 72° C. (preheat the cycler) and place immediately on ice.
26 Prepare the end-repair reaction mix according to your sample number plus extra as multichannel reservoir void volume, as described in Table 2:

TABLE 2

| | Volume (μl) per | Final |
|---|---|---|
| Reagent | reaction | concentration |
| 5x SmartScribe buffer | 4 | 1X |
| 25 mM dNTP (6.25 mM | 2 | 2.5 mM |
| each) | | |
| 100 mM DTT | 1 | 5 mM |
| 100 mM MnCl2 | 0.6 | 3 mM |
| SmartScribe RT enzyme | 1 | |
| Mix Total Volume | 8.4 | |

27 Mix well by vortex, add to a multichannel reservoir and add 8.4 μl of mix to each well (total reaction volume is 20 μl). Mix well by pipetting.
28 Seal the plate with an adhesive PCR film, spin down the plate and incubate 1 h at 42° C., then 15 min at 70° C. in a thermal cycler.

### Multiplexing (Sample Pooling)—10 Min

29 Pool the samples by transferring 19 μl of each sample with a multichannel pipettor into a clean reservoir.
30 Transfer into an Eppendorf low-binding 1.7 mL tube.
   RNase H Treatment (RNA Strand Removal from RNA:cDNA Hybrid to Allow Adapter Annealing During Ligation)—45 Min
31 Add 1 μl of RNase H and incubate in a block set at 37° C. for 20 min.
32 Transfer to another block at 65° C. for 20 min (RNase H inactivation).

### RT Reaction Clean Up—20 Min

33 Add 0.9× of SPRI beads. This ratio of beads to sample should result in a cutoff to remove excess RT primers.
34 Mix well by pipetting 15 times and incubate RT for 2 minutes.
35 Place on the DynaMag-2 magnet for 4 minutes and remove the supernatant.
36 Wash—Add just enough 70% ethanol to cover the beads without removing the tube from the magnet and wait 30 sec.
37 Perform a second wash (repeat step 36).
38 Move the tube off the magnet and leave open for 4 minutes to allow full ethanol evaporation.
39 Add 21 μl of elution buffer (10 mM Tris-HCl pH8.0) and mix well by pipetting 25 times.
40 Let stand for 2 minutes.
41 Place on the magnet for 4 minutes.
42 Transfer 19 μl to a clean PCR well.

### Adapter Ligation—20 Min

43 To the 19 μl cDNA add 29 μl 2× Quick DNA Ligase buffer, 5 μl 5 μM TLA and 5 μl NEB Quick DNA ligase.
44 Close the tube, mix vortex, spindown and transfer to a thermal cycler at 25° C. for 15 minutes.

### Adaptor Ligation Cleanup—20 Min

45 Add 70 μl SPRI beads for 1.5× (ligation buffer already contains PEG) cleanup. With this cutoff the ligation product is retained and free adapter oligo removed.
46 Resuspend beads thoroughly and incubate RT for 2 minutes.
47 Place on the magnet for 4 minutes and remove the supernatant.
48 Wash—Add 100 μl 70% ethanol without removing from the magnet and wait 30 seconds.
49 Perform a second wash (repeat step 48).
50 Move the tube off the magnet and leave open for 4 minutes to allow full ethanol evaporation.
51 Add 13 μl of elution buffer and mix well by pipetting 25 times.
52 Let stand for 2 minutes.
53 Place the tube back on the magnet for 4 minutes.
54 Transfer 11.5 μl to a new PCR well.
   PCR for Library Completion and Enrichment—about 1 h
55 Add 12.5 μl Kapa HiFi ready-mix and 1 μl of 25 μM primer mix (12.5 μM each primer).
56 Close the tube, mix by short vortexing, spindown and incubate in a thermal cycler using the program described in Table 3:

TABLE 3

| Cycle number | Denature | Anneal | Extend |
|---|---|---|---|
| 1 | 98° C., 2 min | | |
| 12-15 | 98° C., 20 sec | 55° C., 30 sec | 72° C., 1 min |
| 16 | | | 72° C., 10 min |

### PCR Reaction Clean Up—20 Min

57 Add 25 μl EB and 40 μl of SPRI beads (0.8×SPRI). Mix 15 times and let stand for 2 minutes.
58 Continue with the SPRI cleanup as described and elute the pooled library with 20 μl EB.
59 Resuspend beads thoroughly and incubate RT for 2 minutes.
60 Place on the magnet for 4 minutes and remove the supernatant.
61 Wash—Add 100 μl 70% ethanol without removing from the magnet and wait 30 seconds.
62 Perform a second wash (repeat step 48).
63 Move the tube off the magnet and leave open for 4 minutes to allow full ethanol evaporation.
64 Add 20 μl of elution buffer and mix well by pipetting 25 times.
65 Let stand for 2 minutes.
66 Place the tube back on the magnet for 4 minutes.
67 Transfer 18 μl of the pool of libraries to a fresh 1.7 mL tube.

### Library Concentration and Size Estimation—15 Min

68 Calculate concentration by Qubit DNA HS.
69 Assess mean library size (in base pairs) by tapestation (average size in bp).

### Example 2

### Comparing the Transeq Method with Template Switch Method

To assess the efficiency of each method, the following parameters were measured:

1. library concentration;
2. Library size and distribution*: a sample of the library was run in a tapestation, a device that replaces bioanalyzer;
3. Efficiency (QC) by measuring the levels of gene expression and how much it is amplified by the PCR step. The expression of the gene Actb (which encodes for beta-actin) was measured.

*to assess size of the library, i.e. the DNA fragment length distribution, which also serves to detect possible unexpected products.

Results

For template switch (TS), 500 ng of total RNA extracted from mouse tissue was used. After 18 cycles of PCR library amplification, a library concentration between 18 and 19 ng per μl in 20 μl library was obtained, and an Actb gene signal enriched by PCR corresponding to 5 to 6 PCR cycles (this corresponds to a 32 to 64× amplification) with respect to the Actb signal after the RT/TS reaction.

For testing Transeq, 200 ng of total RNA from the same sample was used. After 15 cycles of library amplification, ˜9 ng per μl library in 20 μl library was obtained, with an Actb enriched by ˜8.5 cycles=360× amplification.

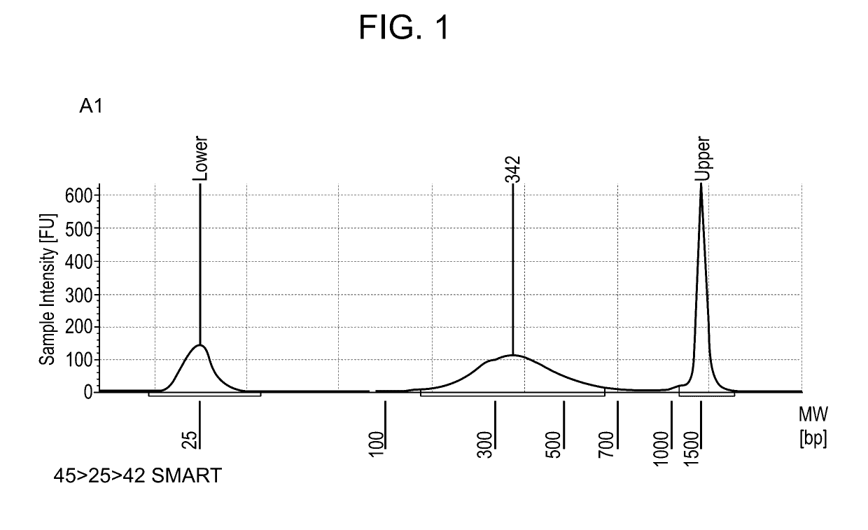

An exemplary library produced by the template switch method is illustrated in FIG. 1 (TapeStation™ profile).

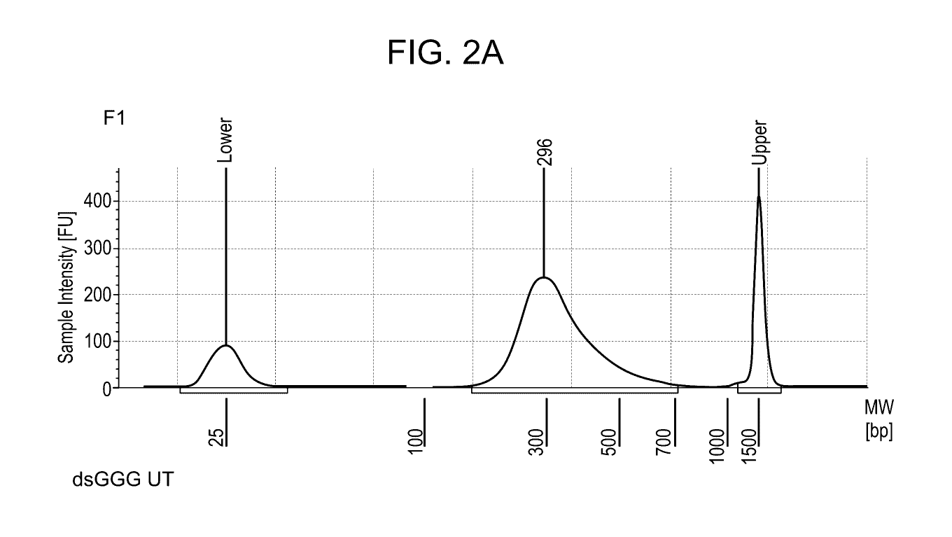

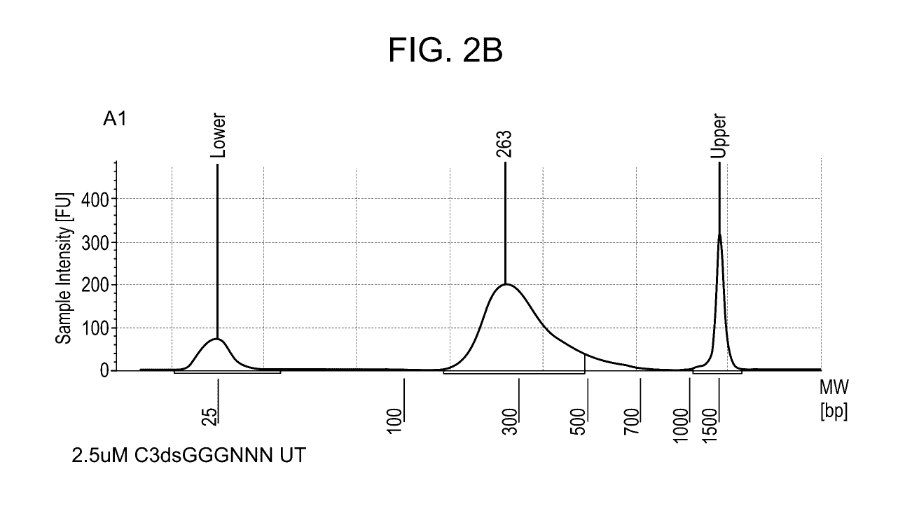

Two exemplary libraries produced by the Transeq method are illustrated in FIGS. 2A-B. As can be seen from FIGS. 2A-B, the peaks are much narrower than those in FIG. 1. Further, the library size distribution is more uniform. Lower and Upper indicate lower and upper internal markers in the TapeStation™ lane.

### Example 3

### Single Cell Transeq (scTRANSEQ)

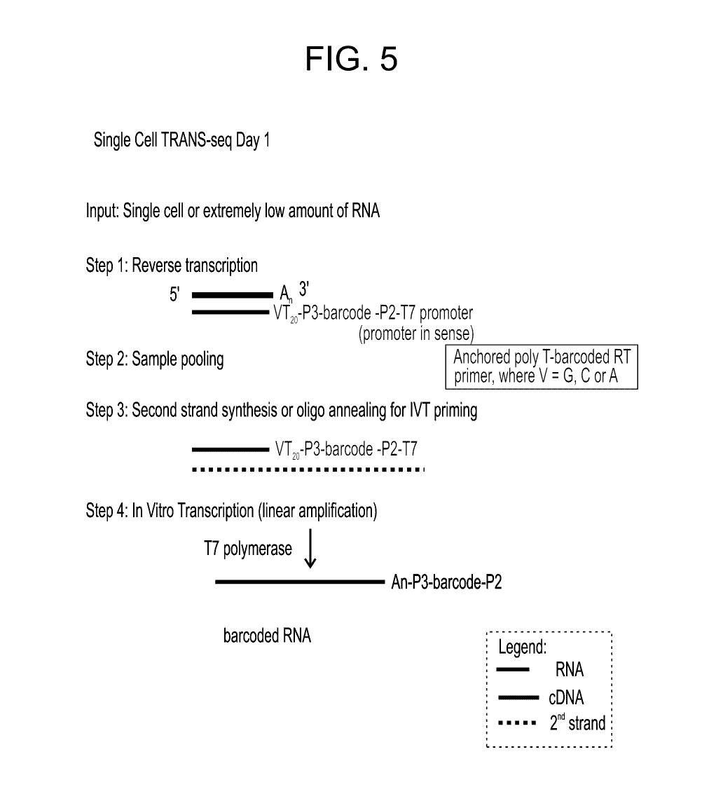

Amplification of samples is represented in FIG. 5.

For single cell transcription profiling, individual cells are first collected, for example by FACS sorting, into a 96-well PCR plate, which contains a mild lysis buffer and the scTRANSEQ reverse transcription (RT) barcoded primer. This primer begins with a T7 RNA polymerase promoter sequence, and contains also adapter sequences required for sequencing.

After collection cells are immediately frozen at −80° C. to enhance cell lysis by a freeze/thaw cycle.

After thawing, lysed cells are heated to open secondary RNA structures, allowing annealing of the RT primer. Next, an RT reaction mix is added to each well. This first RT reaction, RT #1, is performed with a RT enzyme devoid of Tdt activity, and will synthesize cDNA from mRNA ending with a polyA tail. Note that the RNA is not previously fragmented and full mRNA molecules are expected to be reverse transcribed.

Following RT #1, samples are pooled together using a multichannel reservoir, and the cDNA is purified and concentrated using magnetic beads. Next, the second strand is synthesized in one reaction (pooled sample). Then the pooled sample is in-vitro transcribed (IVT), a linear amplification step that generates several copies of RNA per dsDNA molecule using the T7 promoter and the T7 polymerase enzyme. In this way, the low amount of mRNA transcripts per individual cell is highly amplified and reconverted to RNA with the addition of sequencing adapters, a sample barcode to identify the cell (same as in regular TRANSEQ), and a molecular barcode to identify each original molecule in the cell.

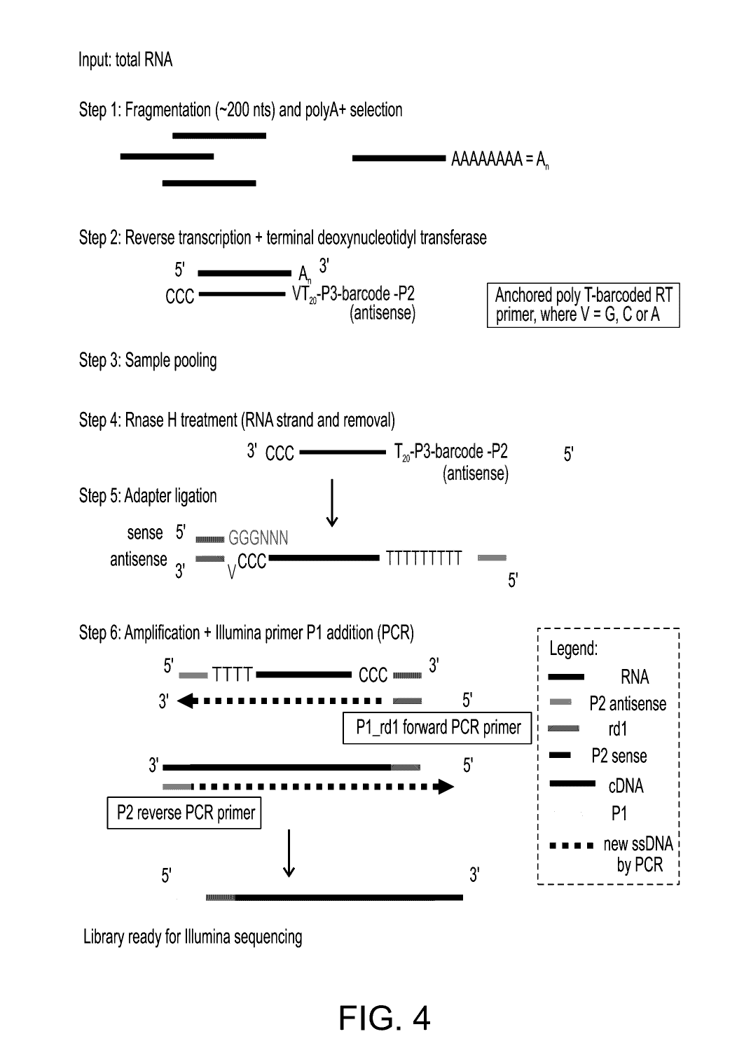

From this stage on, the protocol resembles the regular TRANSEQ scheme as described in Example 1 and illustrated in FIG. 4: the RNA resulting from IVT is fragmented and fragments containing the Illumina adapter and barcodes are newly selected using the polyA selection magnetic beads system. The following step is a second RT reaction (RT #2) using an MMLV RT enzyme with Tdt activity. In contrast to regular TRANSEQ, in this case the RT primer is common to all samples (which are already barcoded). The remaining enzymatic steps are the same as in TRANSEQ, i.e. RNase H treatment, ligation and PCR.

### Example 4

### Comparing the Transeq Method with DGE Method

The present inventors compared the Transeq described herein with standard DGE on different samples.

The DGE method outline: Day 1: create cDNA from RNA, then dsDNA; Day 2: polish the dsDNA to blunt ends, add tailing for ligation, ligate indexed Illumina adapters, PCR).

Results

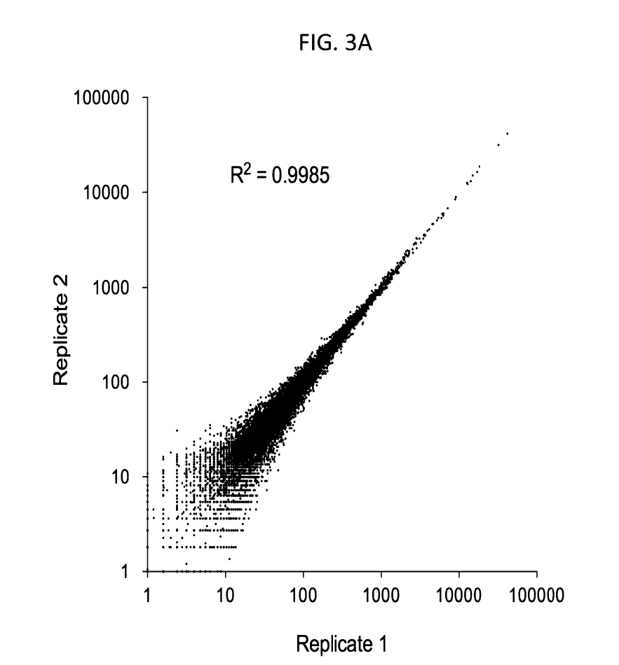

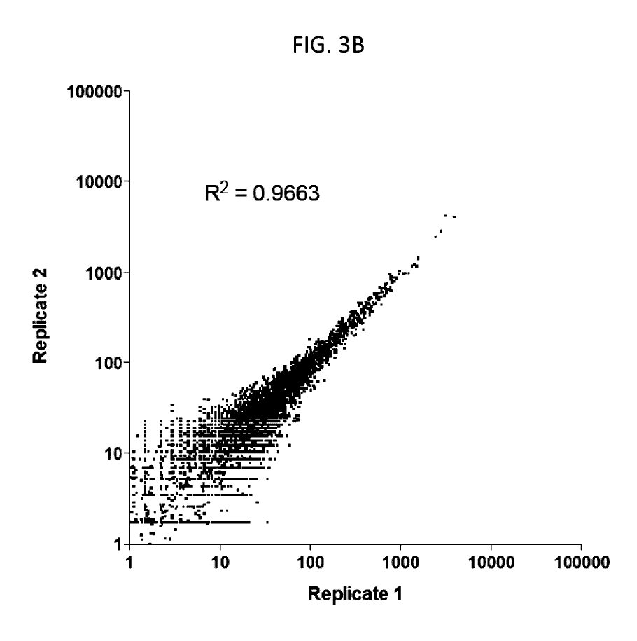

The dynamic range was analyzed for the Transeq method (FIG. 3A) vs. DGE (FIG. 3B). It will be appreciated that for the Transeq method a longer dynamic range is obtained. Also, the R̂2 of the linear fit is higher in Transeq. The higher R̂2 in Transeq may be due to (1) the multiplexing and pooling of samples takes place already at the first enzymatic step and/or (2) the lower number of steps in the protocol both reduce the overall technical error.

### Example 5

### Single Cell Transcriptional Profiling of Splenic Tissue

Materials and Methods

Isolation of Splenic CD11c⁺ Cell Suspension:

Spleens were extracted from C57BL/6J female mice (8 to 12 weeks old), dissociated into single splenocytes with a gentleMACS Dissociator (Miltenyi Biotec) and incubated for 5 minutes in red blood cell lysis solution (Sigma). Cells were then washed and resuspended in MACS buffer (2% FBS and 1 mM EDTA in phosphate-buffered saline), and filtered through a 70-μm strainer. A CD11c⁺ fraction was obtained through two rounds (double-enrichment) of separation with monoclonal anti-mouse CD11c antibodies coupled to magnetic beads using a MACS cell separator system (Miltenyi Biotec).

Single Cell Capture:

Single cells were sorted into cell capture plates, containing 5 μl cell lysis solution for 96-well plates, or 2 μl for 384-well PCR plates. Capture plates were prepared with a Bravo automated liquid handling platform (Agilent). Sorting was performed using a FACSAria III cell sorter (BD Biosciences) and gating in SSC-A vs. FSC-A to collect live cells, and then in FSC-W vs. FSC-A to sort only singlets. Immediately after sorting, plates were spun down to ensure cell immersion into the lysis solution, snap frozen on dry ice and stored at −80° C. until further processing.

Single-Cell/Single-Molecule Barcoding and IVT Amplification (See FIG. 10):

Single cells were collected into a hypotonic cell lysis solution consisting of 0.2% Triton X-100 (a robust splenic lysis solution compatible with our cell direct RT reaction) supplemented with 0.4 U/μl RNasin Plus RNase inhibitor (Promega) and a barcoded RT primer. The RT primer included a T7 RNA polymerase promoter, a partial Illumina paired-end primer sequence, a cell barcode followed by a unique molecular identifier (Kivioja et al., Nat Methods 9, 72 (January, 2012)), and an anchored polydT: CGATTGAGGCCGGTAATACGACTCACTATAGGGGCGACGTGTGCTCTTCC GATCTXXXXXXNNNNTTTTTTTTTTTTTTTTTTTTV (SEQ ID NO: 114, where XXXXXX=cell barcode, NNNN=UMI and V=A, G or C. After thawing, the cell capture plate was incubated at 72° C. for 3 min to open secondary RNA structures and allow annealing of the RT primer. Next, 5 μl or 2 μl Superscript III (Invitrogen) RT reaction mix (10 mM DTT, 4 mM dNTP, 5 U/μl RT enzyme in 50 mM Tris-HCl (pH 8.3), 75 mM KCl, 3 mM MgCl2) were added to each well of the 96-well or 384-well plate, respectively. The RT reaction mix was supplemented with ERCC (Baker et al., 2005, Nat Methods 2, 731). RNA Spike-In mix (Ambion), containing polyadenylated RNA molecules of known length and concentration, at a final 1:40×10⁷ dilution per cell, following the manufacturer guidelines to yield ˜5% of the single cell mRNA content. The plate was incubated 2 min at 42° C., 50 min at 50° C. and finally 5 min at 85° C., after which samples were pooled together into a 1.7 ml low DNA bound microcentrifuge tube (Eppendorf). From this step on all 96/384 samples are treated in a single tube. To remove RT primer leftovers, 1 μl exonuclease I (New England Biolabs) was added to the pool and incubated 30 min at 37° C. and then 20 min at 80° C. for inactivation. The cDNA was purified using paramagnetic SPRI beads (Agencourt AMPure XP, Beckman Coulter) at a 1.2× ratio (to further remove primer traces) and eluted in 17 μl Tris HCl pH7.5. Next, the cDNA was converted to double stranded DNA using a second strand synthesis kit (New England Biolabs) in a 20 μl reaction, incubating for 2 hours at 16° C. The product was purified with 1.4 volumes of SPRI beads, eluted in 8 μl and in-vitro transcribed (with the beads) at 37° C. overnight for linear amplification using the T7 High Yield RNA polymerase IVT kit (New England Biolabs). Finally, the DNA template was removed with Turbo DNase I (Ambion) 15 min at 37° C. and the amplified RNA (aRNA) was purified with 1.2 volumes of SPRI beads.

Single Cell Library Preparation for High-Throughput Sequencing (See FIG. 10):

The aRNA was chemically fragmented into short molecules (median size ˜200 nucleotides) by incubating 2.5 min at 70° C. in Zn²⁺ RNA fragmentation solution (Ambion) and purified with two volumes of SPRI beads. Next, a partial Illumina Read1 sequencing adapter was single strand ligated to the fragmented RNA using a T4 RNA ligase I (New England Biolabs). The aRNA (5 μl) was preincubated 3 min at 70° C. with 1 μl of 100 μM ligation adapter; then, 14 μl of a mix containing 9.5% DMSO, 1 mM ATP, 20% PEG8000 and 1 U/μl T4 ligase in 50 mM Tris HCl pH7.5, 10 mM MgCl2 and 1 mM DTT was added. The ligated primer sequence is: AGATCGGAAGAGCGTCGTGTAG (SEQ ID NO: 115), modified with a phosphate group at 5′ and a 3′ blocker (C3 spacer). The ligated product was reverse transcribed with Superscript III (Invitrogen) and a primer complementary to the ligated adapter (TCTAGCCTTCTCGCAGCACATC; SEQ ID NO: 116). The library was completed and amplified through a nested PCR reaction with 0.5 μM of each primer and PCR ready mix (Kapa Biosystems). The forward primer contained the Illumina P5-Read1 sequences (AATGATACGGCGACCACCGAGATCTACACTCTTTCCCTACACGACGCTCT TCCGATCT—SEQ ID NO: 117) and the reverse primer contained the P7-Read2 sequences (CAAGCAGAAGACGGCATACGAGATGTGACTGGAGTTCAGACGTGTGCTC TTCCGATCT—SEQ ID NO: 118). The amplified pooled single cell library was purified with 0.7 volumes of SPRI beads to remove primer leftovers. Concentration was measured with a Qubit fluorometer (Life Technologies) and mean molecule size was determined with a 2200 TapeStation instrument (Agilent Technologies). Libraries where sequenced using an Illumina HiSeq 2000/2500, 100-1000 samples per lane.

Isolation of DC Subpopulations by Fluorescence-Activated Cell Sorting:

For sorting DC subpopulations, MACS-based CD11c-enriched mouse splenocytes were stained and sorted on a FACSAria III cell sorter (BD Biosciences) in two rounds, using fluorophore-conjugated antibodies (BioLegend). First, cells were stained with FITC-conjugated anti-CD8a antibodies (clone 53-6.7) and sorted into CD8a positive and negative fractions. The CD8⁺ fraction was then stained with APC anti-CD11c (clone N418), Pacific Blue anti-MHCII (clone AF6-120.1), Alexa 700 anti-CD4 (clone GK1.5), PE-Cy7 anti-CD86 (clone GL-1), and PE-conjugated anti-PDCA1. The CD8⁻ fraction was stained for CD11c, MHCII, and with PerCP-Cy5.5 anti-CD11b, PE-Cy7 anti-CD4, FITC anti-PDCA1, and PE-conjugated anti-ESAM (clone 1G8). The DC cells were identified as: cDC CD8⁺ (CD11c^{high} MHCII⁺ CD8a^{high} CD86⁺); cDC CD86⁻ (CD11c^{high} MHCII⁺ CD8a^{inter} CD86⁻); CD8⁺ pDC (CD11c^{inter} CD8a⁺ PDCA1⁺); cDC CD4⁺ ESAM⁺ (CD8⁻ MHCII⁺ CB11b⁺ CD4⁺ ESAM⁺); CD8⁻ pDC (CD11c^{inter} CD8a⁻ PDCA1⁺). For single cell sequencing, single cells were sorted into 96/384 well single cell capture plates as described above.

Isolation of Different Hematopoietic Cell Types:

To obtain B cells, NK cells and monocytes, a splenocyte suspension was stained with, PE-Cy7-conjugated CD19, eFluor 450-conjugated NK-1.1, PerCP Cy5.5 Gr1, FITC TCR-β, APC CD11b and PE B220 (CD45R). B220⁺ and B220^{neg} (germinal center) B cells were collected by gating for CD19⁺ (TCR-β^{neg}) cells and then by B220 against the CD19 marker. NK single cells were collected from the CD19^{neg}/TCR-β^{neg} events by gating for NK-1.1 positive events in NK-1.1 vs. Gr1. Finally single monocytes were collected by gating for Gr1⁺ CD11b⁺ events. The B cell and pDC content in the CD11c-enriched sample was estimated by staining with PE-Cy7 CD19, PE PDCA-1 (CD317, Bst2) and APC CD11c and gating in CD19 vs. CD11c and PDCA-1 vs. CD11c, respectively. For single cell sequencing, single cells were sorted into 96/384 well single cell capture plates as described above.

Single-Cell Real Time PCR:

B, NK and monocyte single cells were sorted by FACS into individual wells of a 96 well plate containing 5 μl of 0.2% Triton X-100 and RNase inhibitor as described above. RT pre-amplification was performed on 24 single cells of each type similarly to Dalerba, et al.(36). After thawing, each well was supplemented with 0.1 μl of SuperScript III RT/Platinum Taq (Invitrogen), 6 μl of 2× reaction mix and a mixture of primer pairs for CD37 (B cell marker), Ly6A (B cell marker), NKg7 (NK marker) and Ccl4 (NK cell marker) genes (100 nM final concentration; primer sequences will be provided upon request). Single-cell mRNA was directly reverse transcribed into cDNA (50° C. for 15 min, 95° C. for 2 min), pre-amplified for 14 cycles (each cycle 95° C. for 15 sec, 60° C. for 1 min) and cooled at 4° C. for 15 min. Samples were then diluted 1:40 with 10 mM Tris-HCl, pH 8. Real-Time PCR analysis was performed for each gene separately with the same set of primers used in the RT pre-amplification stage (400 nM final concentration) using SYBR green Master (Roche) on a LightCycler 480 System instrument (Roche). Quantification was performed as relative to the average of all cells for a given gene (n=72), using the formula 2^{(Ct-mean (Ct))}, where Ct is the mean qPCR cycle threshold signal of two replicate qPCR reactions per cell.

Structure of Valid Library Products and their Expected Distributions:

Following final amplification, single cell RNA-seq sequenced products are structured in two parts. At one end (R1) the present inventors read a 50 bp sequence that should map onto a fragment within some transcribed poly-A gene. For valid library products, this fragment is expected to map at some typical (short) offset from the genes' 3′ UTR, depending on the randomized fragmentation of the initial IVT products during our protocol. The other sequence end (R2) contains a 10-14 bp tag that is engineered to include a 6 bp cell-specific (or well-specific) label, followed by 4-8 bp random molecular tag (RMT). Importantly:

1. Groups of reads that share a cellular tag and RMT are assumed to be representing the same initial RNA molecule and are counted only once. Typically such reads will map to several positions around the 3′UTR of the gene, since following IVT, multiple products sharing the same tag are fragmented variably.

2. The present cell-specific labels are designed to be well separated (in terms of edit distance), reducing the probability of inter-cell contamination through sequencing errors. RMTs, on the other hand, are distributed randomly (and unevenly) over all possible DNA k-mers, making sequencing errors difficult to detect or correct (see below).

3. When deep-sequencing a single cell library, a variable number of reads are expected to cover each RMT. The sequencing depth per molecule mostly depends on its ligation yield and PCR efficiency, which are expected to be similar between molecules that map to the same genomic position. It is therefore expected that molecules representing the same gene and same offset to be covered relatively uniformly and can use such uniformity assumption for normalization.

4. RMTs mark unique molecules with high probability. However the probability of observing two distinct molecules labeled by the same RMT is not zero, especially for genes that are highly expressed. 8 bp RMTs reduce this effect considerably.

Initial Filtering, Tag Extraction and mRNA Sequence Mapping:

Given raw sequenced reads, the present inventors first extract cell-specific tags and RMTs and eliminate reads with ambiguous cell-specific tag. Following this initial filtering the present inventors map R1 reads to the mouse mm9 assembly using the Bowtie program and the standard parameters “-m 1 -t --best --chunkmbs 64-strata”.

They defined a set of transcription termination sites (TTS) by downloading chromosomal coordinates from the UCSC genome browser (mm9) (Meyer et al., 2013, Nucleic Acids Res 41, D64). Sequence reads mapping to a range of −1000 to +200 bp from a known TSS are considered for further analysis. This leaves out of the analysis less than 20% of the sequenced products, likely representing non-classical genes, alternative 3′UTRs, or spurious transcripts.

Following this procedure, they generate a table containing for each cell and each gene, the number of reads covering each of the RMTs in each of the observed mapping offsets. This table is then further processed to eliminate biases and errors.

Filtering RMT Sequencing Errors:

As outlined above, sequencing errors introduced when reading the random Molecular Tags (RMT) in the present library products may undermine the tag-counting approach by creating spuriously identified molecules from real molecules. The number of such spurious RMTs is expected to scale linearly with the number of times each real RMT is sequenced. However, RMT sequencing errors are incapable of changing the offset of the mapped read relative to the TTS, and for each spurious RMT they expect to identify the source RMT as a highly covered tag sharing all the offsets of the spurious RMT.

Based on these assumptions the present inventors developed the following greedy filtering procedure, applied separately for the set of reads assigned to a certain gene/cell pair:

Sort the RMTs given their number of unique mapping offsets.

Repeatedly selecting the RMT T observed at the fewest offsets, and testing if there exist a source RMT S, which is a) observed at all the offsets of T and b) has an edit distance of 1 from T. If such a source RMT exists, T and its associated reads are eliminated.

Identifying and Filtering Skewed Offsets and Cross-Cell Contaminations:

Minimizing cross-cell contamination is important for any single cell RNA-seq pipeline, but is becoming particularly critical when scaling up the protocol to a large number of cells and when applying it to a heterogeneous sample. Even relatively small levels of read to cell association errors can create a strong background and batch effect, increase spurious correlations between cells and reduce the capability of the approach to detect small coherent subpopulations. In theory, contamination is prevented by well-specific labeling, since the latter is retained following pooling of material from single cells and throughout the different stages of the protocol. Nevertheless, the extensive PCR amplification performed during library construction, and the existence of common (poly-T) sequences at one end of the library products may give rise to unexpected scenarios of “tag-switching” and read mislabeling. The present inventors therefore studied the complex distributions of reads over cells, genes, 3′UTR offsets, and RMTs in our data, aiming to identify and eliminate such potential noise factors.

Given data on a group of cells c=1 . . . m from the same amplification batch, read counts r(c, o, T) are defined at each offset o=−1000 . . . +200 and RMT T. Naively, each RMT/cell pair represents a distinct molecule, which may be observed at several offsets. They denote the number of offsets at which at least one read was observed for a pair c,T as n(c,t). They also compute the set of presumed molecules c,T that are sequenced at least once in an offset o, denoted M(o). It can be expected that whenever M(o) represent many real molecules that were pre-amplified through IVT and then fragmented, these molecules (indicated by their cell tag and RMT) will also be occurring at other offsets. Specifically, the distribution of n(c,T) for pairs (c,T) in M(o) is expected to scale with ∥M(o)∥. They defined the offset skew for any value of o on a certain gene as the ratio between ∥M(o)∥ and the median of n(c|T) values across M(o).

Empirically it was observed that the offset skew is rarely bigger than 2. On the other hand it was observed that for specific offsets and genes, the number of molecules is very high, although almost all occurrences of the molecule are observed only at that offset, generating a very high offset skew. Importantly, it was noted that this effect is highly specific to amplification batches, suggesting it is indeed an amplification artifact. Since offset skew increase coverage on specific genes for specific amplification batches they studied the correlation between single cell RNA-seq profile over different batches to establish a filtering heuristic that minimize batch-dependent expression. Following optimization over 4 different CD11c⁺ amplification batches, the policy was set to filter reads on offsets with skew bigger than 1.8 and ∥M(o)∥>=3. In addition, all offsets for which the median number of reads per RMT was 1 over five or more molecules were filtered. It was noted that filtering reads on a particular offset does not imply that the RMTs on that offsets are all discarded, since their appearance on other, non-skewed offsets, is expected for valid molecules.

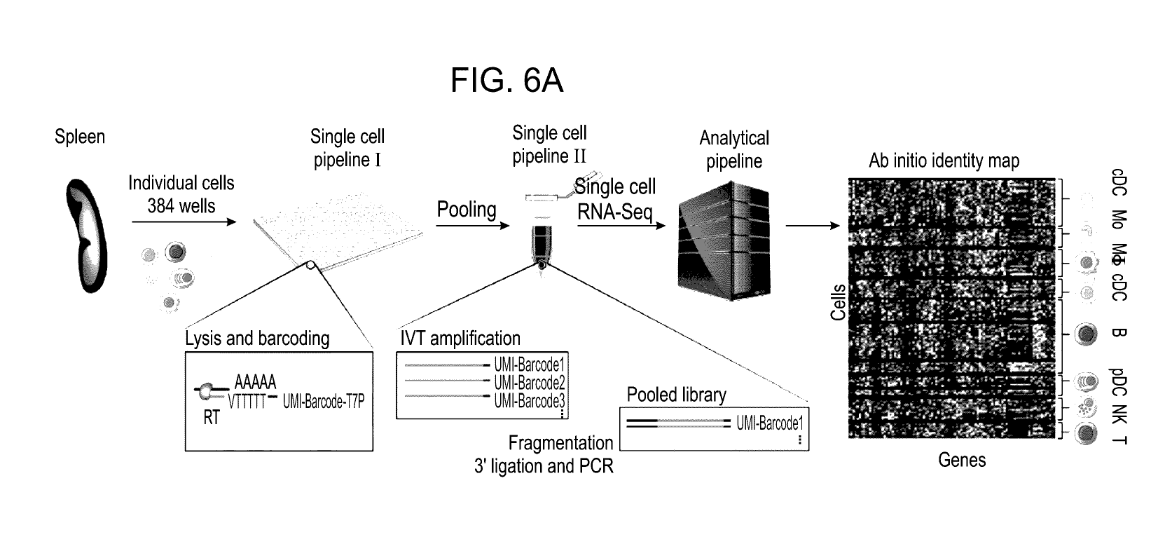

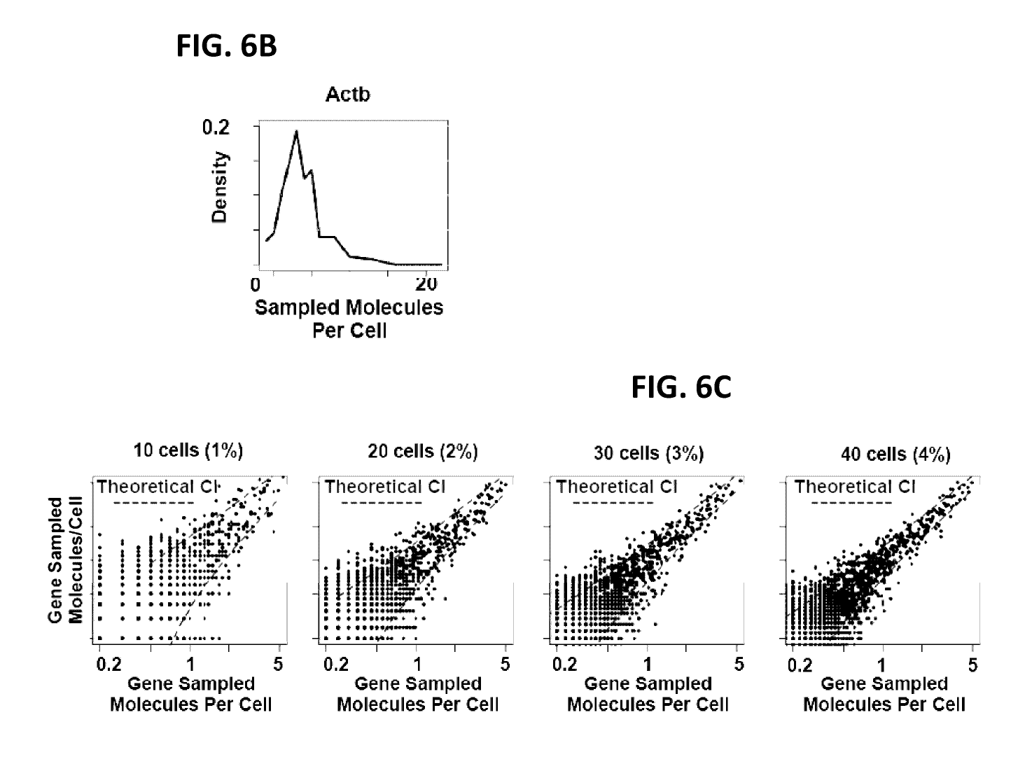

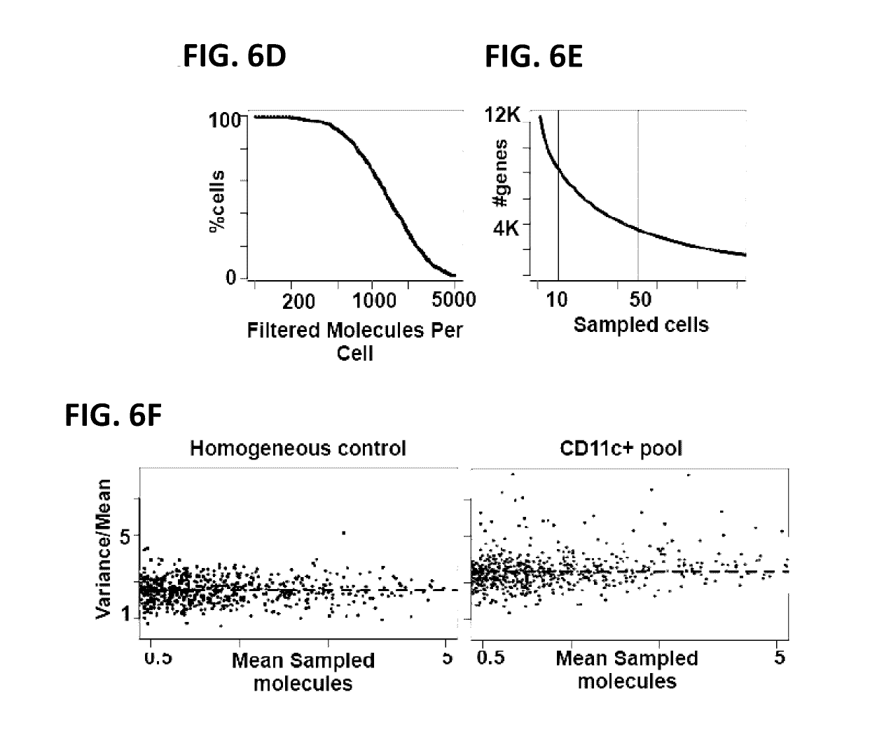

Following filtering of RMT sequencing errors and offset skews the data is relatively free of batch specific effects, and provides very predictable distributions of coverage and reproducibility (as shown in FIGS. 6A-F and the subpopulations results).

Down-Sampling Normalization:

Unlike any other gene expression datasets, the present single cell profiles are inherently discrete, representing samples from the pools of RNA molecules within each cell. The number of trustworthy sampled molecules per cell is variable and in order to compare profiles between cells, some normalization must be performed. Since the present samples can be truly considered as multinomial samples from each cell, the only appropriate normalization scheme is probabilistic—they define a target number of molecules N, and then sample from each cell having m>=N molecules precisely N molecules without replacement. Cells with m<N are not used for comparisons at this level. This down-sampling approach ensures that all normalized cells should reflect the same multinomial distribution and can be compared robustly. It should be noted that common practices in normalization of gene expression data (e.g. dividing by mean or median) must be avoided in single cell RNA-seq datasets as they introduce severe coverage biases to the analysis.

A Multinomial Mixture Model for Single Cell RNA-Seq Data:

To model a single cell dataset, the following simple multinomial mixture model is introduced. The model probabilistically generates vectors of mRNA molecule counts over some space of genes G. Some number of classes K (analogues to the number of clusters, currently estimated manually) are assumed. Each class is defining a multinomial distribution over G, pᵢⱼ=Pr(gⱼ|class=i) (the probability of sampling gene j given we are in mixture i) and a mixture coefficient aᵢ. The probability of a single cell mRNA sample that is defined by a vector nⱼ (number of molecules observed for gene j) is defined by summation over all multinomial probabilities (Πpᵢⱼ^{nj}) weighted by the mixture coefficients.

To initialize the model parameters the following procedure is performed:

Hierarchical clustering on downsampled single cell profiles with coverage>=1500.

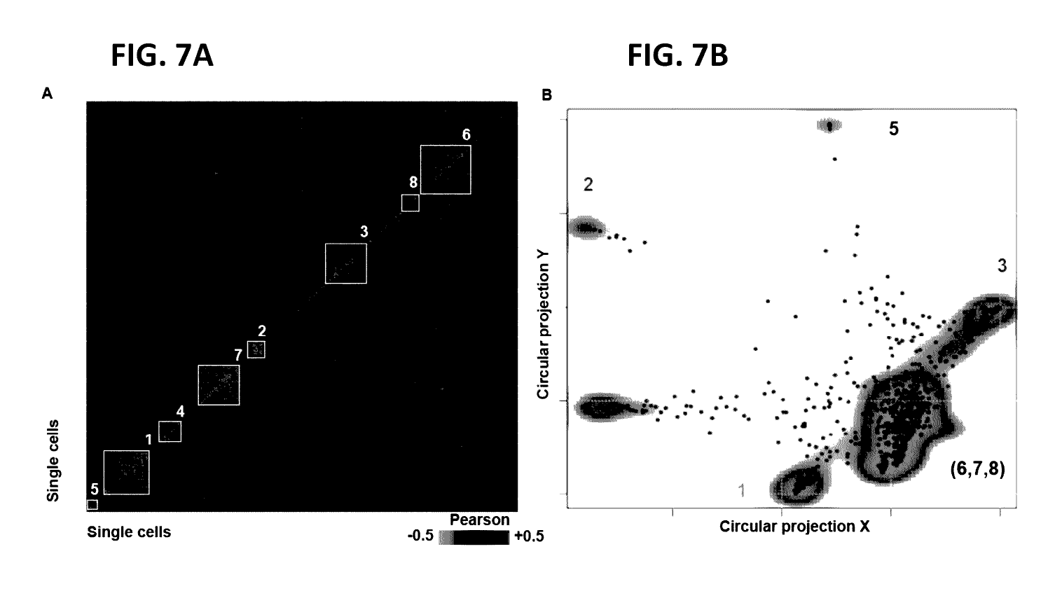

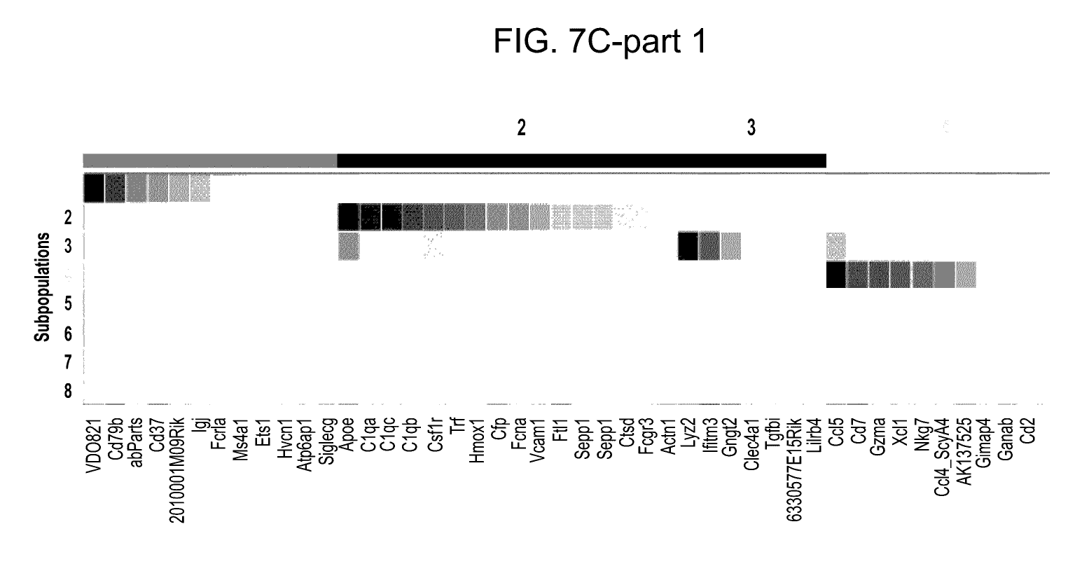

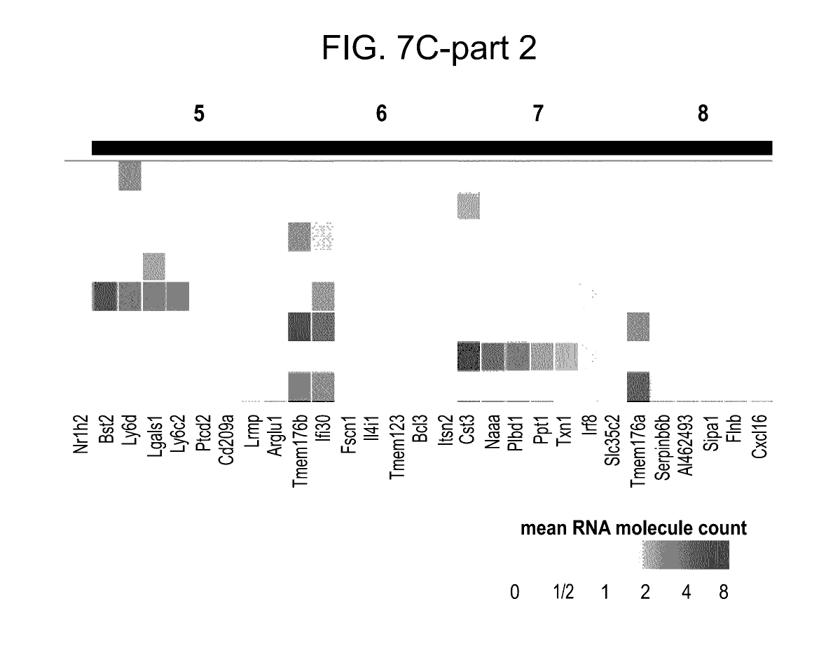

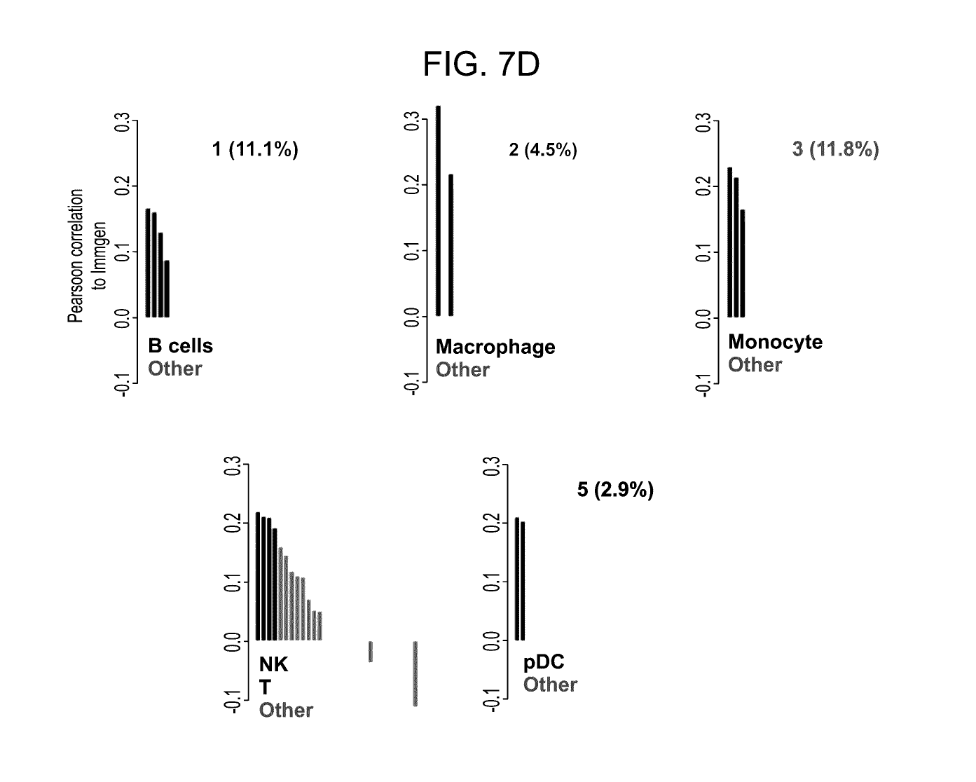

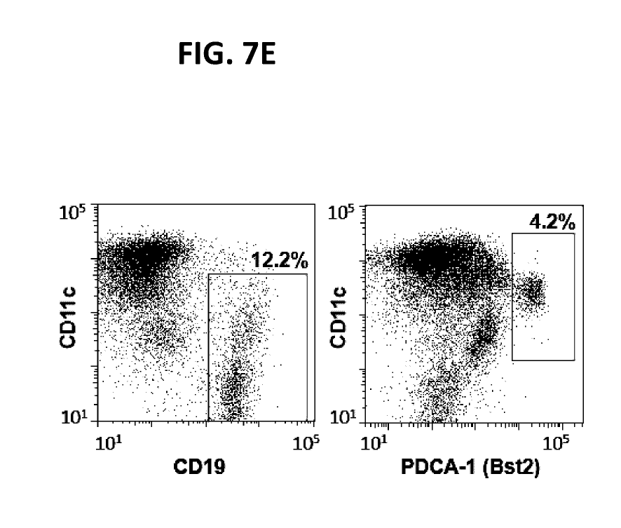

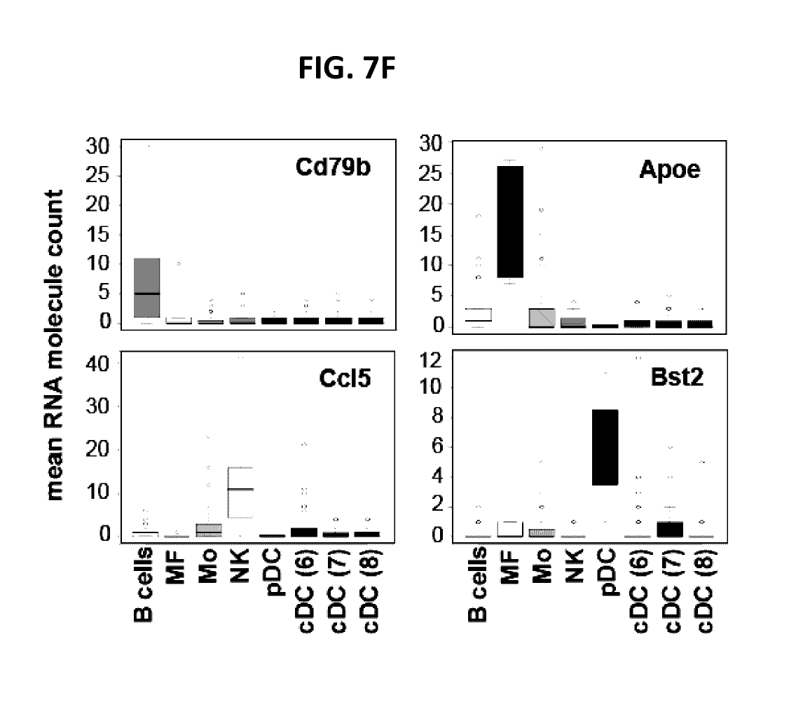

Identification of high correlation sub-trees, i.e. seeds. For the analysis of FIGS. 7A-F the present inventors have manually selected only three of the potential DC seeds.

Initializing pᵢⱼ by pooling together all molecules from cells in the j seed, followed by down-sampling of a fixed number of molecules (9600 for the CD11c⁺) without replacement, and estimation of the multinomial probabilities from the resulted set, adding regularization counts of 1 molecule for all genes and estimating the multinomial probabilities from the resulted set.

The present inventors currently substantiate selection of the parameter K of our class seeds by further comparison of the model to sorted libraries and extensive gene expression datasets.

They suggest that intrinsic, machine-learning theoretic validation of the model is currently premature as are more sophisticated learning approaches, given that the data generation and filtering procedures are still not sufficiently mature.

Circular a-Posteriori Projection (CAP-) Visualization:

While the above probabilistic model is initialized from down-sampled single cell profiles, the model is applicable to any set of molecules. Given a collection of (non down-sampled) single cell profiles nᵢ^{k} (specifying the number of molecules for gene i in cell k), standard probabilities for each class uⱼₖ=Pr(n^{k}|class j) may be computed. These values are standardized to avoid introducing a coverage bias and then normalized over all class j to generate a corrected posterior probability: u′jₖ=exp((100/sumⱼ(nⱼ^{k})*log(uⱼₖ))/Zₖ (where Zₖ is the normalization factor). To visualize this high dimensional data a circular projection is defined by assigning each class with a radial position αⱼ on the unit circle, and assigning each cell with a coordinate x=sumⱼ (u′jₖ cos(αⱼ)), y=sumⱼ (u′jₖ sin(αⱼ)).
Radial positions are selected to minimize the inconsistencies for cells with ambiguous class posteriors. Specifically, pairs of classes with many cells mapping ambiguously to them should be positioned on proximal radial positions. To find an assignment of radial positions, a complete graph over the cells is constructed, and traveling salesman problem is solved over this graph with distances that represent the inverse number of cells with strong joint posterior probability for each pair of classes. Specifically they compute the joint posterior matrix by multiplying the posterior matrices V=U′U′^{T}, normalize the product v′_{jj′}=v_{jj′}*(1/sumₗ(uⱼₗ)*sumₗ(u_{j′l})) and generate a distance dᵢⱼ=exp(−10vᵢⱼ).

Pooling Subpopulation RNAs and Testing Differential Expression:

To test differential gene expression between groups of single cells, the present inventors performed a standard chi-square based proportion test on cases for which at least 6 molecules are observed for cells within the class. They corrected p-values for multiple testing using Benjamini Hochberg procedure (FDR<0.05).

To compare the present data to previously established microarray based gene expression signatures from the ImmGen project, normalized expression vectors of the following representative ImmGen classes were used: SC.LTSL.BM, SC.STSL.BM, PROB.FRBC.FL, PREB.FRD.FL, B.FO.SP, B.GC.SP, B.MZ.SP, B1A.PC, DC.8+.TH, DC.4+.SP, DC.8+.SP, DC.PDC.8+.SP, DC.PDC.8-.SP, DC.LC.SK, MF.RP.SP, MF.THIO5.11-480HI.PC, MO.6C+II-.BL, MO.6C+II+.BL, MO.6C+II-.LN, GN.BM, NK.SP, NK.49CI-.SP, NK.49CI+.SP, NK.MCMV1.SP, T.DPSM.TH, T.4NVE.SP, T.4MEM.SP, T.4FP3+25+.SP, T.8NVE.SP, T.8MEM.SP.OT1.D45.VSVOVA, T.8MEM.SP.OT1.D106.VSVOVA, TGD.TH, TGD.SP, TGD.VG5+.ACT.IEL and compute correlated to the present cells over genes with mean mRNA count per cell >0.1.

Results

Massively parallel single cell RNA-seq to sample cells from a mouse spleen was applied. Cells were coarsely enriched for DCs using a cell surface marker (CD11c⁺) antibody coupled to MACS magnetic beads. Through this semi-biased approach the present inventors interrogate a heterogeneous sample, while maintaining a focus on the splenic DC population whose internal structure and functional compositions are still not fully understood. 891 CD11c⁺ cells were assayed, deriving between 500-4000 distinct molecules per cell (FIG. 6D), and RNA from 8000 genes in 10 different cells or more and 4000 genes in 50 cells or more was recovered (FIG. 6E). Importantly, it was observed that the variance of mRNA counts in a control set of biologically homogeneous cells (FACS-sorted pDC) is scaling tightly with the mean expression (FIG. 6F). In contrast, data for the biologically more heterogeneous CD11c⁺ population, showed a globally higher variance vs. mean trend, as well as the existence of many individual high variance genes. This suggested that groups of variably expressed (or potentially co-varied) genes within the CD11c⁺ population can be used to de-novo identify its functional cell type composition based solely on the mRNA counts of single cells and using no prior assumptions.

Unsupervised clustering of the cell's filtered and standardized expression counts (FIG. 7A) showed that indeed, and as suggested by the existence of high-variance genes, the population is structured into groups of highly correlated transcriptional states. To characterize these states in a principled fashion that is compatible with the experiment, the present inventors employed a probabilistic mixture model emitting discrete vectors of molecular counts. This approach naturally models the present experimental process (i.e. sampling labeled molecules from single cell RNA pools) and allows inference of the mixture model parameters directly from the data. The population was visualized by circular a-posteriori projection (CAP) of the model classification predictions onto a two-dimensional space (Methods and FIG. 7B). Remarkably, the CD11c⁺-enriched splenic population is dissected by this approach into 8 subpopulations of varying sizes and mRNA count profiles.

To further explore the functional cell states detected by this completely unbiased bottom-up approach, the mean mRNA count profile of each subpopulation was studied (selected genes are shown in FIG. 7C), and compared to previously established gene expression profiles of marker-based sorted hematopoietic cells from diverse lineages (FIG. 7D). This led to unequivocal association of four subpopulations with B cells (11.1%), macrophages (4.5%), monocytes (11.8%) and pDC (2.9%), and an additional subpopulation with strong association to NK and T cell markers (5.8%). The remaining three single cell subpopulations defined a transcriptional state correlated with the mean DC behavior. The subpopulation frequencies estimated from the single cell RNA-Seq data were validated using measurements obtained from FACS sorting of the CD11c⁺ MACS enriched population using the relevant marker for each predicted population (FIG. 7E). It was also confirmed that known lineage specific genes (e.g. CD79b, ApoE, Csf1r, Cc15, NKg7, Bst2/PDCA-1), are robustly enriched in their relevant subpopulation (FIG. 7F) and further validated this data using single cell qPCR. Together, analysis of the CD11c enriched cell population demonstrates that single cell RNA-seq can be used to classify a heterogeneous cell sample into functionally coherent groups without prior marker selection and based on de novo characterization of subpopulations with gene rich transcriptional profiles.

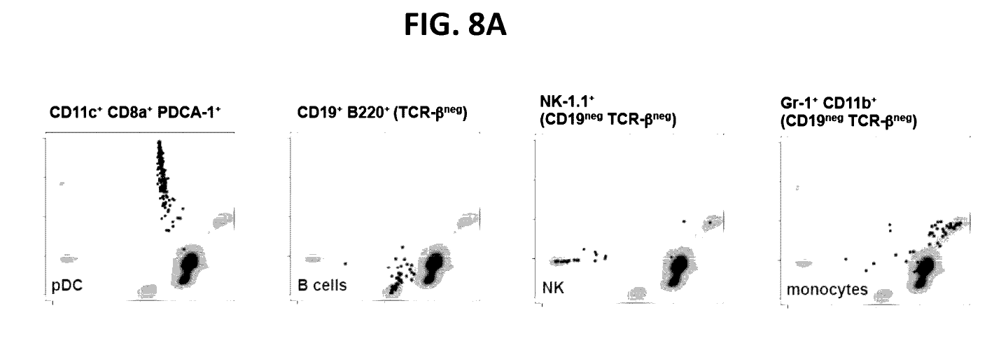

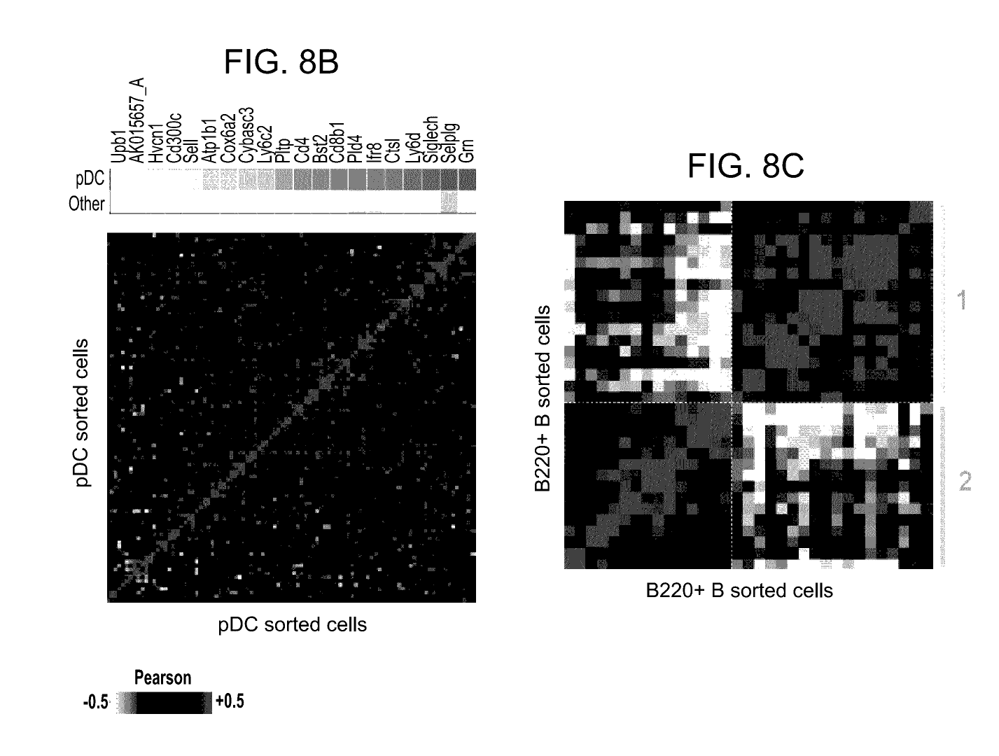

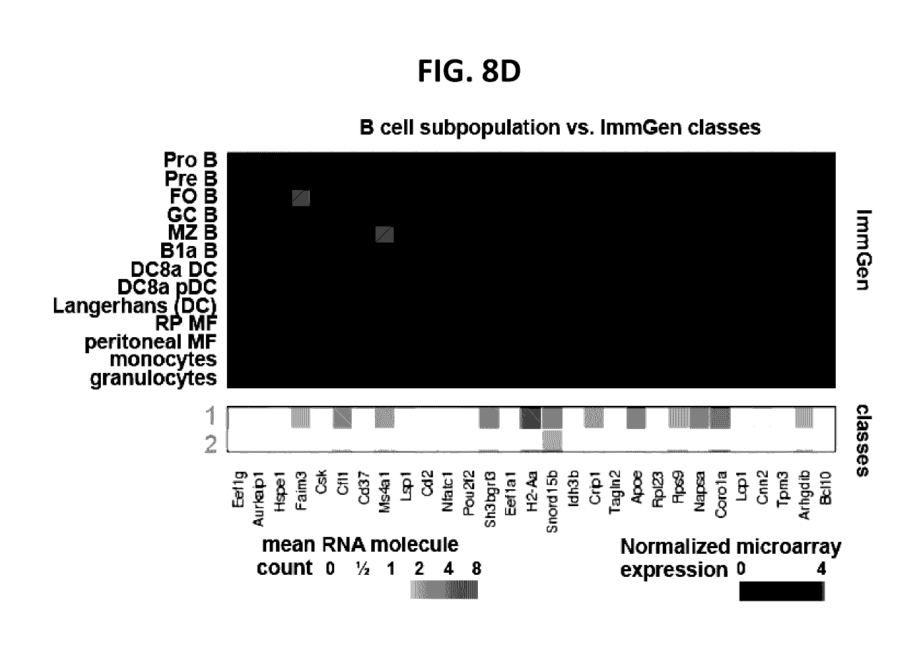

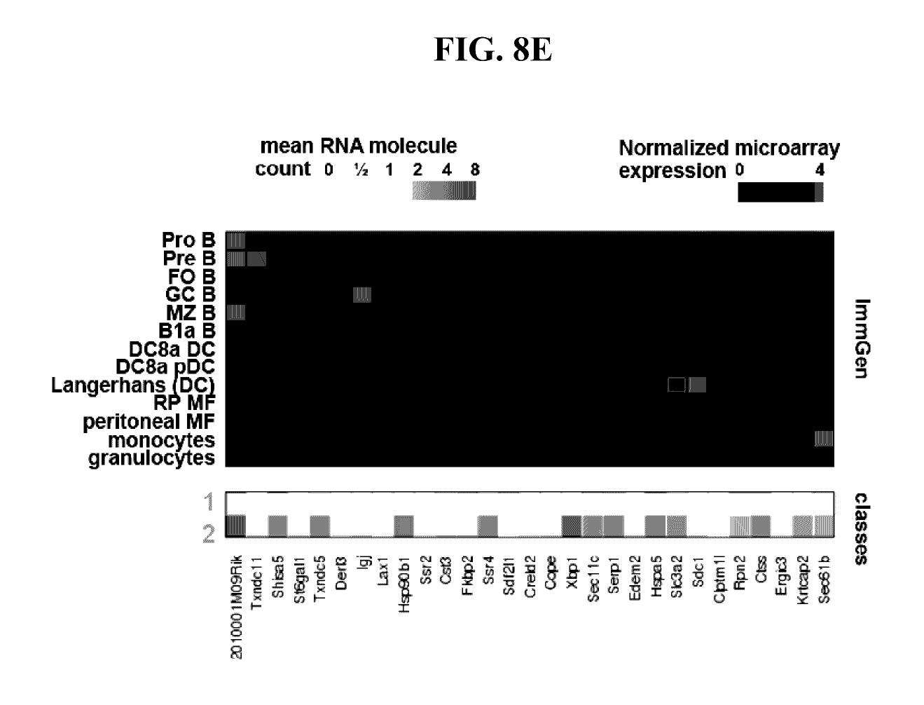

To confirm the present results and better understand how immune cell populations at different levels of homogeneity would look at the single cell level, the present inventors generated additional single cell panels from conventionally FACS sorted populations of NK cells, pDCs, monocytes and B cells. Projection of the FACS sorted single cells onto the CD11c⁺ mixture model reconfirmed the identity of its subpopulations, while suggesting different degree of heterogeneity within them (FIG. 8A). As it was clear that the large but limited sample that was used for analyzing the CD11c⁺ population covers only a small fraction of the functional diversity within the immune system, the present inventors further studied the substructure of the FACS sorted subpopulations. Importantly, the pDC FACS-sorted population (FIG. 8B) showed lack of significant internal correlation structure, despite being distinguished from other populations by multiple pDC specific genes (FIG. 8B). This high degree of homogeneity in the pDC functional state provides an interesting observation on the coherence of the pDC gene regulation program. In addition these data serve as an important negative-control for the present assay, showing it is not enforcing subpopulation structure in populations that are as homogeneous as pDCs. In marked contrast to pDCs, functionally rich and diverse populations such as NK cells or B cells (FIG. 8C) are significantly sub-structured. For such rich populations it is possible to identify multiple co-expressed genes that characterize the emerging subpopulations functionally. In the B cell data, the present inventors distinguished a subpopulation of cells expressing genes like Faim3, ApoE, and Pou2f2, from cells expressing Igj, Xbp1 and proliferation related genes (FIGS. 8D-E). Interestingly, comparison of the transcriptional profile to ImmGen cell types (FIGS. 8D-E) show that many of the genes separating the B cells subpopulations are not necessarily B-cell specific. This indicates that functional sub-class definition can be achieved through combinations of multiple low specificity genes rather than by separation using specific markers. In summary, sequencing of FACS sorted single cell populations validates our functional sorting paradigm, and confirm the expectation that deeper sampling of specific immunological niches as B cells will lead to characterization of finer substructure within them that are difficult to separate using marker based approaches.

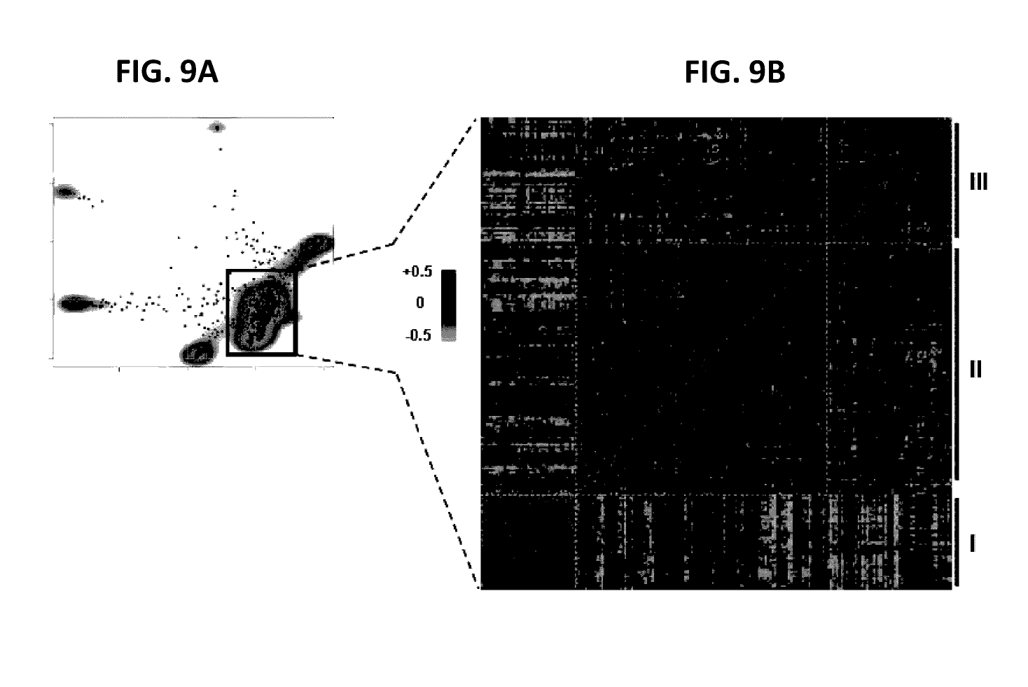

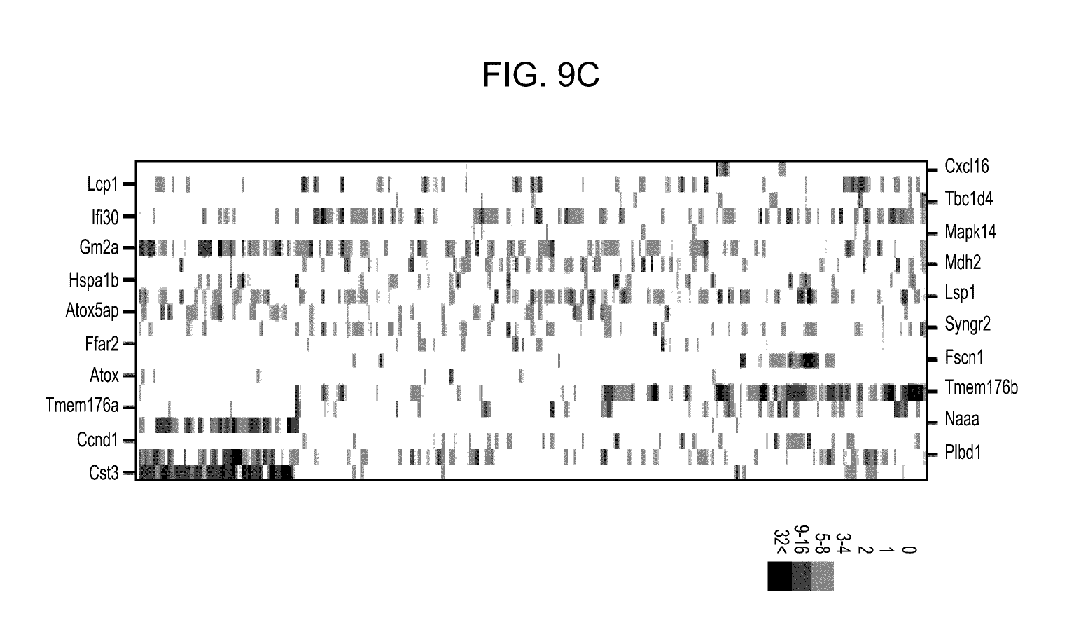

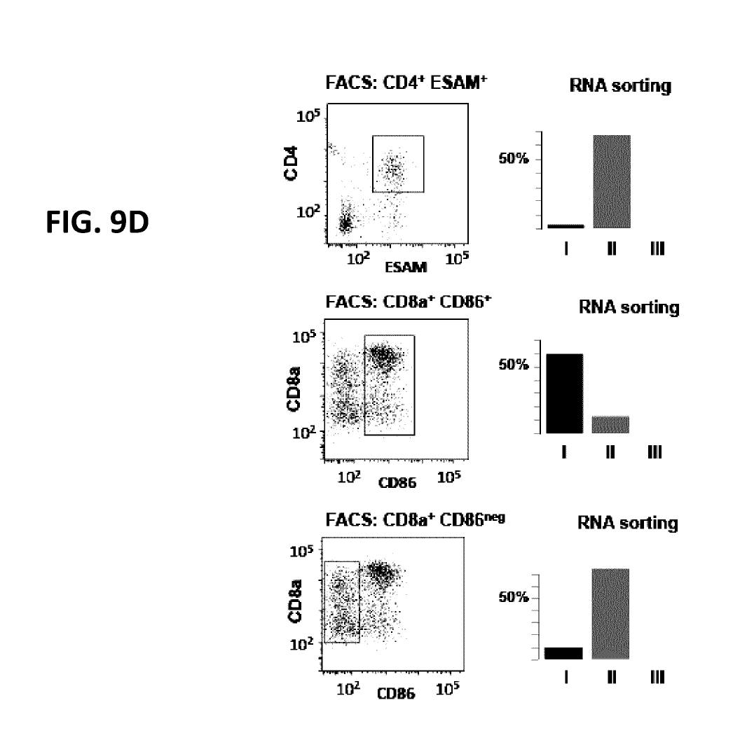

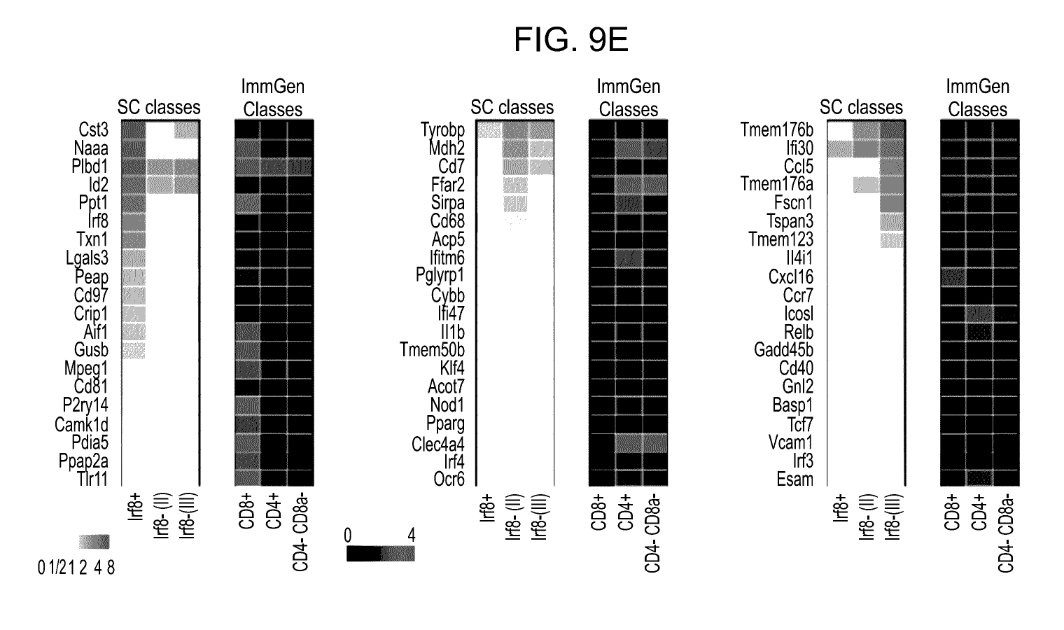

After validating that the present functional sorting approach is compatible with conventional FACS when applied to cell populations originating from distinct and well-separated hematopoietic lineages, the more challenging population of DCs was analyzed. DCs are extensively studied using multiple cell surface markers and reporter assays, but current data provide variable, and sometime contradicting models for the activity and differentiation states of the splenic DC reservoir. Functional re-sorting of all CD11c⁺ single cell profiles that were associated with classical DC (cDC) classes (FIG. 9A) led to identification of three broad cDC functional groups (class I-III, FIG. 9B). To rule out the possibility that this analysis identified non-DC subgroups, the present inventors confirmed that genes that were identified as related to the cDCs classes in the global analysis of the CD11c⁺ pool (e.g. Itgax, Plbd1, Cst3, Flt3) are consistently expressed in cells from all three classes (FIG. 9C), and that non-DC marker genes detected in the CD11c⁺ pool are not significantly enriched in these classes. To understand these classes in the context of previously gold-standard marker based sorted DC cell populations, three additional single cell RNA-seq datasets were generated from a CD8^{high} CD86⁺ population, a CD8^{inter} (intermediate) CD86⁻ population and a CD4⁺ ESAM⁺ population. Mapping the resulted single cell profiles onto the DC mixture model showed clearly that the CD8^{high} CD86⁺ pool is enriched for class I states (˜60%), but also contain significant representation of class III (˜24%) and class II (16%) states (FIG. 9D). Conversely, the CD4+ population was highly enriched for class II (71%), with significant class III representation (˜28%) and low representation of class I (FIG. 9D). These observations associated class I with previously defined CD8⁺ DC and class II with previously defined CD4⁺ DC. Interestingly, the CD8^{inter} CD86⁻ population showed strong class II enrichment (73%) with residual class I representation (12%), suggesting that in fact CD8^{inter} CD86⁻ DC are significantly different from their CD8^{high} CD86⁺ DC counterpart as reported previously. These analyses are underlining the power of the unbiased approach facilitated by single cell RNA-sequencing. Using no prior assumptions or thresholds, the single cell profiles define functional states based on hundreds of genes in a way that is fully comparable between experiments and batches. No marker selection is performed, and therefore the derived functional states that can be readily overlaid over one universal functional landscape. It is possible that additional subpopulation structure is hidden inside the classes described above, but such structure must involve correlations among transcriptional states that are weaker than those defined by the primary classification described here.

By pooling together sampled single cell transcriptional profiles of the three broad DC subpopulations defined above, the present inventors were next able to study unprecedentedly pure and rich transcriptional programs in DCs. They identified 767 genes that were differentially expressed between the three classes (at FDR<0.05), including numerous regulators, surface markers, cytokines and more. Class I cells (FIG. 9E) are defined by co-expression of Irf8 (known to regulate the CD8⁺ DC state (26)) and Id2, together with a large set of genes including many signaling molecules (e.g. Tlr11) and surface markers (CD8a, Cd24a and Cd81). Class II cells are defined by weak enrichment of Irf4 and Klf4 expression (26, 29) (FIG. 9E) and a second distinct set of signaling molecules and surface markers (Sirpa, Clec4a, Ccr6). These two classes provide a de novo unbiased characterization of the CD8⁺ and CD4⁺ DC transcriptional states, which can be assumed to be more accurate than FACS-based profiles averaging a transcriptionally mixed cell pool (FIG. 9D). Class III cells (which as noted above are mixed within the CD8⁺ and CD4⁺ sorted DC), are low in Irf8 and Irf4 levels (FIG. 9E). On the other hand, cells within this group express strongly the NfKB members RelB and Nfkb1 as well as the NfKB inhibitor Nfkbia (the latter two are also common to class II cells). Expression of Irf2, Irf3 and Irf5 in this group was also detected, as well as a signature of Ccr7 and CD83. Importantly, the distinct characteristics of each of the three cDC classes described herein do not necessarily imply their homogeneity. In specific, class II and class III cells are relatively weakly defined, showing a remarkably diverse spectrum of transcriptional states. This suggests a high degree of functional plasticity within Irf8⁻ DCs, implying that rigid classification hierarchies within this population may be hard to define in principle. In contrast, the Irf8⁺ populations of cDC and pDC (FIGS. 8B and 9B) appears transcriptionally more homogeneous, raising questions as to the mechanisms regulating such homogeneity and why these are not affecting the Irf8⁻ DCs. These observations set the stage for a new framework for studying DC biology, and are suggesting that mechanisms for controlling the plasticity of the DC transcriptional state may be a regulated process that is controlled differently between DC subtypes.

The present inventors present a new methodology for microscopic analysis of the transcriptional programs in heterogeneous mammalian tissues. Using broad sampling of single cell transcriptional states from multi-cellular tissues they can reconstruct biological function in a bottom-up fashion, starting from its most basic building block—the cell. The present technique is applicable immediately in any molecular biology lab, requires no specific equipment or setup and can provide data on RNAs from hundreds of single cells at the cost of one standard average gene expression profile. This approach overcomes the shortcomings of top-down marker based approaches, circumventing the need to find suitable markers and ensure their robustness across experiments. The method can also resolve the difficulties in adapting cell surface markers and cell types definitions from model organisms to human cells. As demonstrated above, the present inventors have used their framework as a tool that combines cell sorting and functional characterization modalities into one. When applied to hematopoietic subpopulations that are extremely well defined and lineage-separated, this technique replaces laborious, biased and delicate marker-based sorting and gene expression profiling by a process that characterizes eight or possibly more subpopulations in one experiment. In the more challenging setting of exploring the functional structure within complex and multi-faceted DC population, the present methodology leads to clear and unambiguous separation of the DC subpopulations. Marked differences in the heterogeneity of different DC subtypes were observed ranging from the highly homogenous pDC population to the extreme gene expression heterogeneity in the two Irf8⁻ cDC classes. It may be hypothesized that these different levels of gene expression plasticity in DC subpopulations can serve as a key functional feature of these cells, which must respond and adapt to variable environments and challenges and interact extensively with multiple other types of immune cells.

Functional sorting using single cell RNA-seq can be readily applied to numerous tissues and organs. The data emerging for this new microscopic device is likely to challenge present working models of development, differentiation and functional plasticity in health and disease. Extensive unbiased sampling of the transcriptional states of cells in vivo can lead to a real breakthrough in our understanding of multi-cellular biological function. Such function may soon be studied as an emergent property of a complex and stochastic mixture of microscopic states rather than the outcome of a system that is engineered deterministically from relatively few precise functional building blocks. Single cell transcriptional sampling can contribute greatly to narrowing the gap between experimental modeling in vitro and the phenotype in vivo by allowing measurements of cells directly from their in vivo contexts. This aspect of the technology can help building a vital link between modern systematic approaches to biology that deepen our mechanistic understanding toward genome function and regulation, and the highly specific, individualized and complex biological phenomena that are driven by such mechanisms within cells, tissues and organs. With many thousands of single cell functional profiles within easy reach, the stage is set for this long sought-after development.

### Example 6

### Massively Parallel RNA Single Cell Sequencing Method (MARS-Seq)—Automation Set-Up

Automated single cell RNA-Seq library production is performed on the Bravo automated liquid handling platform (Agilent) using 384-filtered tip (Axygen, catalog #302-82-101). The Bravo Single Cell RNA-Seq scripts are available upon request and can be implanted on other liquid handling robots.

384-Well Cell Capture Plates Preparation Protocol

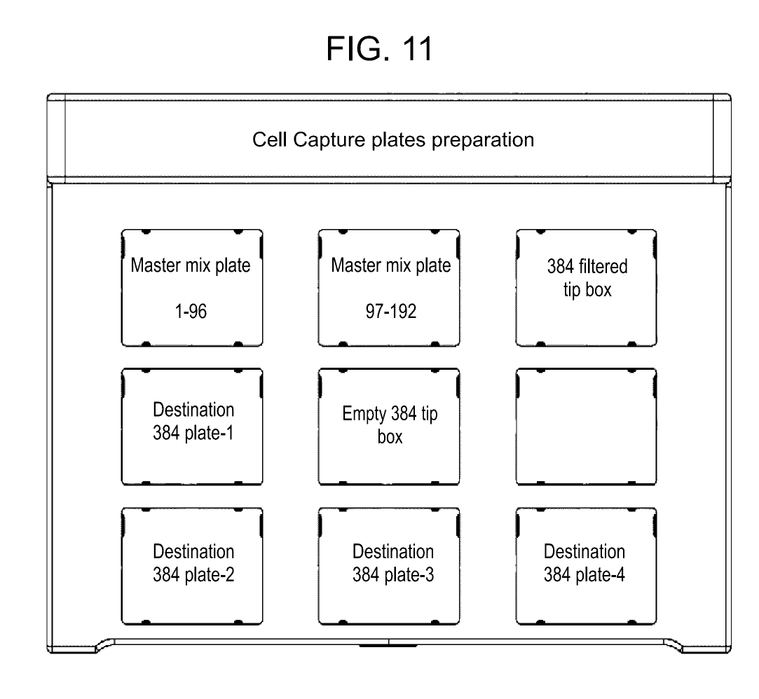

1. 96-well master mix plates contain lysis buffer (triton 0.2% in molecular biology water) supplemented with 0.4 U/μl RNase inhibitor and 400 nM of RT1 primer from group 1 (1-96 barcodes) or group 2 (97-192 barcodes). To prepare 12 384-well plates, 57.5 μl lysis buffer are mixed with 5 μl 5 μM RT1 primer stock per well.
2. The cell capture plate preparation script mixes group 1 master mix plate (barcodes 1-96), aspirates 2 μl from it and dispenses it in destination 384-well plate-1 in two adjacent positions (see below). Then, 2 μl are again aspirated from master mix plate 1-96 to be dispensed in the other destination 384-well plates. If more than four cell capture plates are needed, filled destination plates should be replaced with new plates. Once all desired plates are added with group 1 master mix, tips are replaced and the cell capture plate preparation script mixes group 2 master plate (barcode 97-192), aspirates 2 μl from it and dispenses it in the destination 384-well plates—see FIG. 11. The entire process takes about 30 min per 12 plates. A single cell is then sorted into each well using FACS.

Barcoding and RT Reaction

1. RT reaction mix (10 mM DTT, 4 mM dNTP, 2.5 U/μl RT enzyme in 50 mM Tris-HCl (pH 8.3), 75 mM KCl, 3 mM MgCl₂, ERCC RNA Spike-In mix) is prepared as a mix of 440 reactions (sufficient for 384 wells). The RT mix is divided into two 8 well strips, 54 μl per well, placed in a 4° C. 96-well Inheco stand (below).

2. The RT reaction mix addition script adds 2 μl from the RT reaction mix into the 2 μl of lysis buffer that includes a unique primer and a single cell in the 384-well plate (placed in a 4° C. 384-well Inheco stand) and mixes the reaction one time. Tips are replaced and the process is repeated until all wells in the entire 384-well plate are supplemented with RT reaction mix.

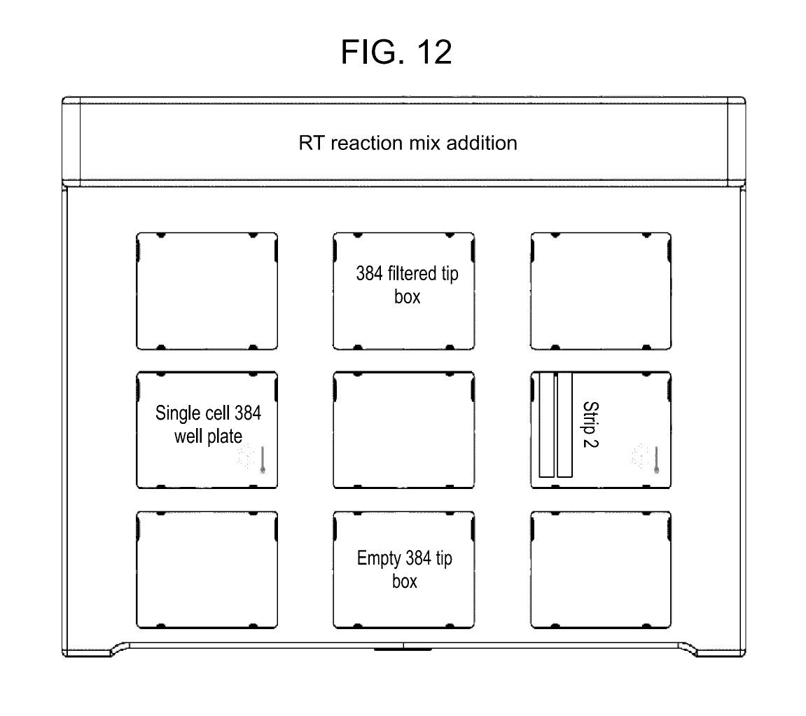

3. The 384-well plate is then spun down and moved into a 384 cycler (Eppendorf) for the RT program (2 min at 42° C., 50 min at 50° C., 5 min at 85° C.,). The entire process takes 23 min per 384-well plate—see FIG. 12.

Pooling of Barcoded 384 Single Cell Samples:

1. Tips pre-wash and blocking: prepare 1 ml triton 0.2%+40 ng yeast tRNA. Dispense 50 μl in each well in row D of a clean 96-well plate (destination plate for pooling).

2. 4 μl of the barcoded cDNA sample from the 384-well plate (placed in a 4° C. Inheco stand) are pooled into two rows (24 wells) in a 96-well destination plate (placed in a 4° C. Inheco stand). The entire process takes 10 min per plate.

3. 1 μl of exonuclease I (NEB) is added into each well in the 2 rows of the 96-well plate and the plate is incubated at 37° C. for 30 min and then 10 min at 80° C. for inactivation.

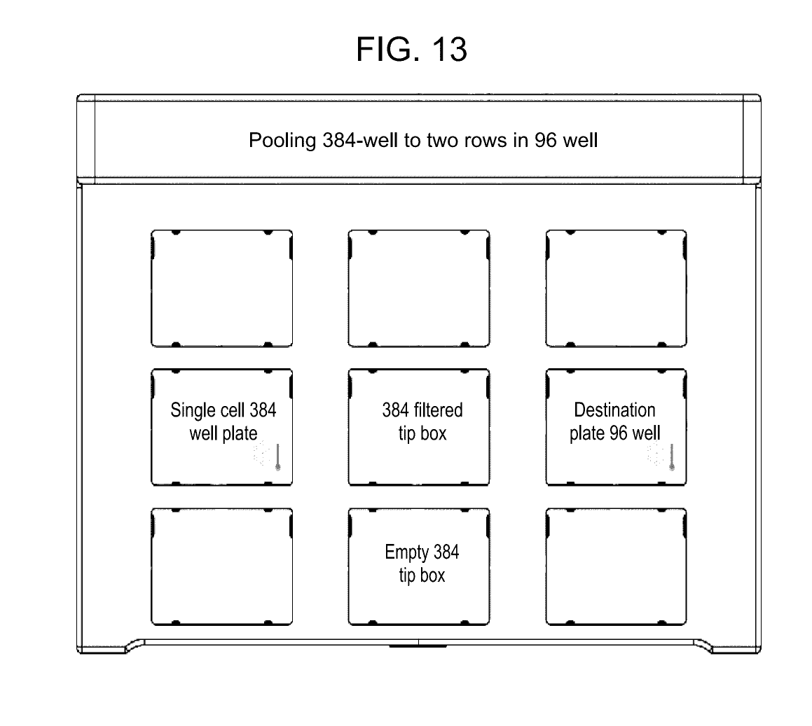

4. 1.2 volumes of SPRI beads are added into each well and the contents of each row (containing 192 barcoded single cells) are pooled into a single DNA non-binding eppendorf tube and purified. These two groups of 192 samples will be pooled together after addition of plate barcode in the RNA-DNA ligation step (below)—see FIG. 13.

### Example 7

Recent years have seen a revolution in genome-wide single-cell transcription technologies, improving sensitivity, throughput and accuracy. In contrast, development of similar technology for meso-scale measurement of a targeted set of tens to hundreds of markers is greatly lacking. The present inventors have developed a novel highly accurate targeted single-cell RNA-seq protocol. This protocol amplifies a user-defined set of transcripts from tens of thousands of individual cells in a simple, accurate and rapid protocol. The method of amplification used enables exact counting of the number of transcripts of each gene in each cell, giving high-accuracy, low cost and high throughput measurements.

Methods

Sorting:

Cell sorting was performed as described [Jaitin, D. A. et al. Massively Parallel Single-Cell RNA-Seq for Marker-Free Decomposition of Tissues into Cell Types. Science 343, 776-779 (2014)]. Spleens from female C57BL/6 mice were extracted and passed through a strainer. Red blood cells were lysed and cells were stained with target antibodies. FACS gates were determined based on unstained controls and the literature.

Primer Design:

Reverse transcription (RT) primers consist of a polyT-anchoring region, a random 4 or 8 bp nucleotide sequence (the UMI), a cellular barcode and adjacent sequences required for in vitro transcription.

Primers for gene multiplexing consist of (from 5′ to 3′) 10 bp of an Illumina adapter (rd1), 10 bp of a universal sequence, 20 bp gene-specific sequence. Candidates for gene specific sequences were designed with primer-blast to fit to a region 200-800 bp upstream of the polyA tail and a Tm of 62±3. Primers were designed so that the lengths of the amplicons would be as similar as possible; this prevents bias due to shorter amplicons being amplified more efficiently.

The reverse primer is common to all genes and is identical to a sequence found on the RT primer.

Molecular Biology:

RT reaction, exonuclease 1 and SPRI cleanups were performed as described previously [Jaitin, D. A. et al. Massively Parallel Single-Cell RNA-Seq for Marker-Free Decomposition of Tissues into Cell Types. Science 343, 776-779 (2014)] with the adjustment of SPRI 1.2× and not SPRI 0.8× after pooling. The multiplex PCR reaction was carried out in 60 μl with 30 μl 2× Kappa HiFi mix, 19 μl template, 11 μl primer mix such that all gene-specific primers have a final concentration of 50-100 nM and the reverse primer has concentration equal to the sum of all gene-specific primer concentrations. PCR program was 98° C. 2 min, [98° C. 20 s, 60° C. 20 s, 72° C. 30 s]×20, 72° C. 5 min. This was followed by SPRI 0.9× and elution in 16 μl. 3 μl were taken into another PCR reaction with 50 μl 2× Kappa HiFi mix and 2 μl enrichment primers (500 nM final each). PCR program was 98° C. 2 min, [98° C. 20 s, 63° C. 20 s, 72° C. 30 s]×10-13, 72° C. 5 min.

Alignment and Filtering:

All reads which started with a perfect match to the universal 10 bp sequence were extracted from the Illumina output files. Reads with a phred score<20 in any of the UMI bases were dumped prior to analysis. Reads were aligned by comparing bases 20-40 to a sequence of 10 bp primer and 10 bp predicted mRNA sequence extracted from the UCSC database. 87±5% of the reads had an exact match, 8±3% had 1 bp difference and <2% had 2-4 bp difference. Reads with more than 4 bp mismatch to a primer-predicted pair but with an exact match to the primer were defined as misaligned. All other reads were defined as unidentified.

After gene-alignment reads were separated into separate cells based on an exact match or a match with 1 error to known cell barcodes. 97% of all aligned reads were successfully separated into their respective cells. UMI filtering was carried out separately for each gene: all UMIs aligned to a given gene were arranged in a list by readcount and a threshold was passed at 16-fold below the second-highest UMI. Alternatively, all UMIs at hamming distance <=2 from a UMI with higher readcount were discarded.

Protocol Design:

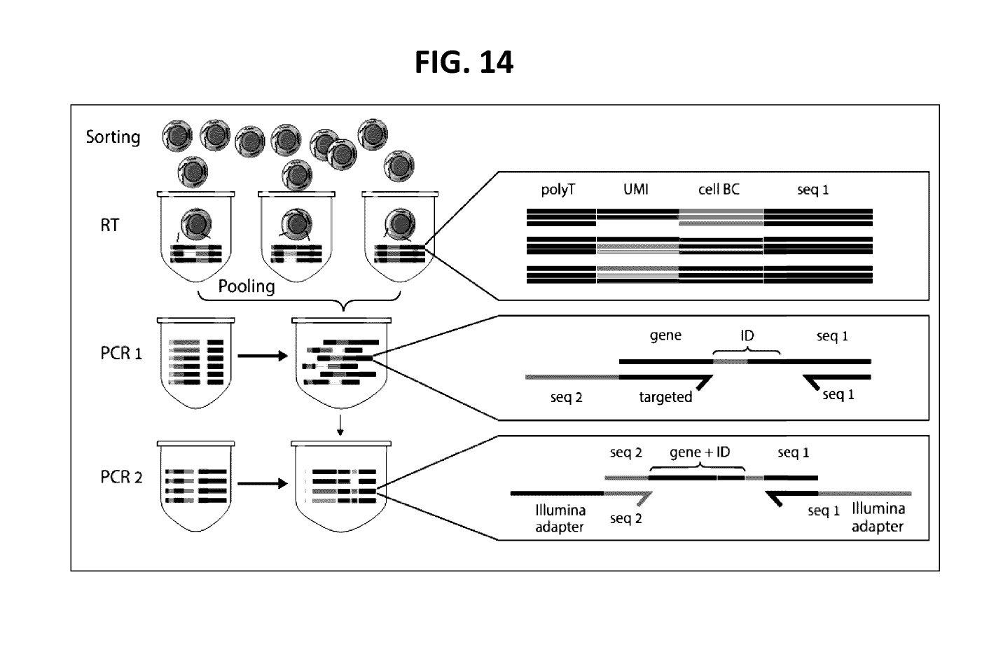

The general design of the protocol is as follows and can be seen in FIG. 14:

1. Reverse transcription with polyT primers

2. Exonuclease1

3. SPRI cleanup

4. Enrichment of targets with gene-specific primers

5. SPRI cleanup

6. Enrichment and addition of Illumina adapters

Results

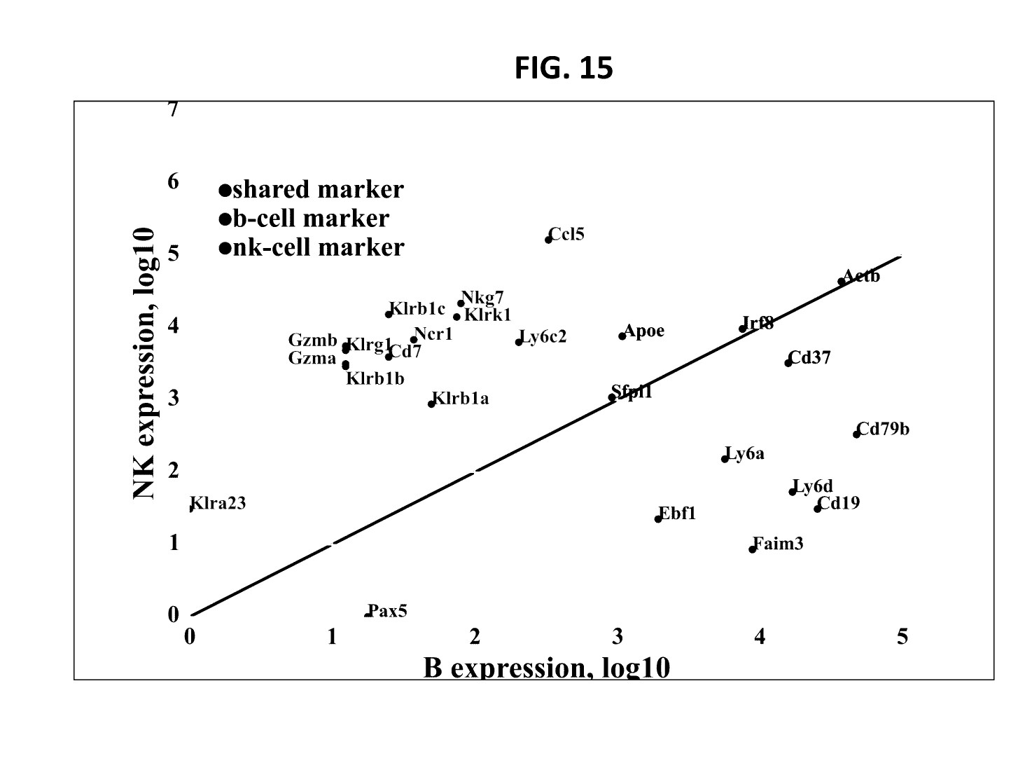

B and NK cells were sorted and a library was created consisting of 24 replicates each of 10 pg and 100 pg from both cell types. From this pool a panel of 25 genes including both cell-specific and common markers from highly expressing and lowly expressing genes was amplified (FIG. 15). Fold-change analysis shows that the protocol can capture at least a 10-fold difference between the same gene in different samples and at least a 100-fold difference between different genes in the same sample. Reproducibility conforms to that predicted by a simple stochastic model.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A kit for transcriptome analysis comprising:
(i) a first oligonucleotide comprising a polydT sequence at its terminal 3′ end, a RNA polymerase promoter sequence at its terminal 5′ end and a barcode sequence positioned between said polydT sequence and said RNA polymerase promoter sequence, wherein said barcode sequence comprises a cell barcode and a molecular identifier;
(ii) a second oligonucleotide being a single stranded DNA having a free phosphate at its 5′end;
(iii) a third oligonucleotide being a single stranded DNA which is fully complementary to said second oligonucleotide.

2. A method of preparing a cell for transcriptome sequencing comprising:
(a) incubating a plurality of RNA molecules with a reverse transcriptase enzyme and a first oligonucleotide comprising a polydT sequence at its terminal 3′ end, a RNA polymerase promoter sequence at its terminal 5′ end and a barcode sequence positioned between said polydT sequence and said RNA polymerase promoter sequence under conditions that allow synthesis of a single stranded DNA molecule from said RNA, wherein said barcode sequence comprises a cell barcode and a molecular identifier;
(b) synthesizing a complementary sequence to said single stranded DNA molecule so as to generate a double stranded DNA molecule;
(c) incubating said double stranded DNA molecule with a T7 RNA polymerase under conditions which allow synthesis of amplified RNA from said double stranded DNA molecule;
(d) fragmenting said amplified RNA into fragmented RNA molecules of about 200 nucleotides;
(e) incubating said fragmented RNA molecules with a ligase enzyme and a second oligonucleotide being a single stranded DNA and having a free phosphate at its 5′end under conditions that allow ligation of said second oligonucleotide to said fragmented RNA molecules so as to generate extended RNA molecules; and
(f) incubating said extended RNA molecules with a third oligonucleotide being a single stranded DNA and which is complementary to said second oligonucleotide, thereby preparing the cell for transcriptome sequencing.

3. The kit of claim 1, further comprising a T4 RNA ligase and/or a reverse transcriptase.

4. The kit of claim 1, wherein said first, said second and said third oligonucleotide are each packaged in a separate container.

5. The kit of claim 1, wherein said second oligonucleotide has a blocking group at its 3′ end.

6. The kit of claim 1, wherein said second and said third oligonucleotide are between 10-50 nucleotides in length.

7. The kit of claim 1, wherein said second and said third oligonucleotide are between 15 and 25 nucleotides in length.

8. The kit of claim 1, wherein said first oligonucleotide is no longer than 100 nucleotides.

9. The method of claim 2, being performed on a plurality of cells within a tissue, wherein said barcode sequence indicates the identity of the cells of the tissue.

10. The method of claim 9, further comprising pooling said single stranded DNA molecules synthesized in step (a), said pooling being effected prior to step (b).

11. A kit for synthesizing cDNA from an RNA sample comprising a library of adapter polynucleotides, a polydT oligonucleotide which is coupled to a barcoding sequence and a reverse transcriptase comprising terminal Deoxynucleotidyl Transferase (TdT) activity, wherein each member of the library comprises a double-stranded DNA portion with a 3′ single stranded overhang, wherein said double-stranded DNA portion comprises 15 base pairs and no more than 100 base pairs, wherein said 3′ single stranded overhang comprises at least 3 bases and no more than 10 bases, wherein said double stranded DNA portion is at the 5′ end of the polynucleotide, wherein the 5′ end of the strand of said double-stranded DNA which is devoid of said 3′ single stranded overhang comprises a free phosphate, wherein the sequence of said double stranded portion of said each member of the library is identical, wherein said sequence of said 3′ single stranded overhang of each member of the library is non-identical.

12. The kit of claim 11, wherein said reverse transcriptase comprises Moloney Murine Leukemia Virus Reverse Transcriptase (MMLV-RT).

13. The kit of claim 11, further comprising at least one of the following components: (i) a ligase; (ii) a DNA polymerase; (iii) MgCl₂ (iv) a PCR primer; and (v) RNase H.

14. The kit of claim 13, wherein said polydT oligonucleotide is attached to a solid support.

15. The kit of claim 11, wherein the 5′ terminus of said polydT oligonucleotide comprises an RNA polymerase promoter sequence.

16. A method for generating cDNA, comprising the steps of:
(a) combining an RNA sample with a polydT oligonucleotide under conditions sufficient to allow annealing of said polydT oligonucleotide to mRNA in said RNA sample to produce a polydT-mRNA complex, wherein a 5′ end of said polydT oligonucleotide is coupled to a barcoding sequence;
(b) incubating said polydT-mRNA complex with a reverse transcriptase comprising terminal Deoxynucleotidyl Transferase (TdT) activity under conditions which permit template-dependent extension of said polydT to generate an mRNA-cDNA hybrid;
(c) contacting said mRNA-cDNA hybrid with Rnase H under conditions which allow generation of a single stranded cDNA molecule; and
(d) incubating said single stranded cDNA molecule with:
(i) an adapter polynucleotide comprising a double-stranded DNA portion with a 3′single stranded overhang, wherein said double-stranded DNA portion comprises 15 base pairs and no more than 100 base pairs, wherein said 3′single stranded overhang comprises at least 3 bases and no more than 10 bases, wherein said double stranded DNA portion is at the 5′ end of the polynucleotide, wherein the 5′ end of the strand of said double-stranded DNA which is devoid of said 3′single stranded overhang comprises a free phosphate and wherein the sequence of said 3′ single stranded overhang is selected such that it is capable of hybridizing to the 3′ end of said single stranded DNA molecule; and
(ii) a ligase enzyme, under conditions which permit ligation of said adapter polynucleotide to said single stranded cDNA molecule, thereby generating the cDNA.

17. The method of claim 16, wherein said polydT oligonucleotide is attached to a solid support.

18. The method of claim 16, further comprising amplifying the quantity of RNA in said RNA sample prior to step (a).

19. The method of claim 18, wherein said amplifying is effected by:
(a) contacting said RNA with a polydT oligonucleotide having a RNA polymerase promoter sequence at its terminal 5′ end under conditions sufficient to allow annealing of said polydT oligonucleotide to said RNA to produce a polydT-mRNA complex;
(b) incubating said polydT-mRNA complex with a reverse transcriptase devoid of terminal Deoxynucleotidyl Transferase (TdT) activity under conditions which permit template-dependent extension of said polydT to generate an mRNA-cDNA hybrid;
(c) synthesizing a double stranded DNA molecule from said mRNA-cDNA hybrid; and
(d) transcribing RNA from said double stranded DNA molecule.


SEQUENCE LISTING (118 sequences)

SEQ ID NO: 1 - DNA, 6 bp, Artificial sequence
  Exemplary contemplated sequence of the 3′ single stranded overhang of the adapter polynucleotide
gggnnn

SEQ ID NO: 2 - DNA, 3 bp, Artificial sequence
  Exemplary contemplated sequence of the 3′ single stranded overhang of the adapter polynucleotide
ttt

SEQ ID NO: 3 - DNA, 6 bp, Artificial sequence
  Exemplary contemplated sequence of the 3′ single stranded overhang of the adapter polynucleotide
tttnnn

SEQ ID NO: 4 - DNA, 3 bp, Artificial sequence
  Exemplary contemplated sequence of the 3′ single stranded overhang of the adapter polynucleotide
ngg

SEQ ID NO: 5 - DNA, 6 bp, Artificial sequence
  Exemplary contemplated sequence of the 3′ single stranded overhang of the adapter polynucleotide
nggnnn

SEQ ID NO: 6 - DNA, 3 bp, Artificial sequence
  Exemplary contemplated sequence of the 3′ single stranded overhang of the adapter polynucleotide
ntt

SEQ ID NO: 7 - DNA, 6 bp, Artificial sequence
  Exemplary contemplated sequence of the 3′ single stranded overhang of the adapter polynucleotide
nttnnn

SEQ ID NO: 8 - DNA, 3 bp, Artificial sequence
  Exemplary contemplated sequence of the 3′ single stranded overhang of the adapter polynucleotide
tgg

SEQ ID NO: 9 - DNA, 6 bp, Artificial sequence
  Exemplary contemplated sequence of the 3′ single stranded overhang of the adapter polynucleotide
tggnnn

SEQ ID NO: 10 - DNA, 6 bp, Artificial sequence
  Barcode sequence
tgcggc

SEQ ID NO: 11 - DNA, 6 bp, Artificial sequence
  Barcode sequence
atataa

SEQ ID NO: 12 - DNA, 86 bp, Artificial sequence
  Indexed RT primer
caagcagaagacggcatacgagatnnnnnnnngtgactggagttcagacgtgtgctcttccgatcttttttttttttttttttttn

SEQ ID NO: 13 - DNA, 28 bp, Artificial sequence
  TRANSeq ligation adapter (Sense strand)
ctacacgacgctcttccgatctgggnnn

SEQ ID NO: 14 - DNA, 22 bp, Artificial sequence
  TRANSeq ligation adapter (Antisense strand)
agatcggaagagcgtcgtgtag

SEQ ID NO: 15 - DNA, 58 bp, Artificial sequence
  Single strand DNA oligonucleotide
aatgatacggcgaccaccgagatctacactctttccctacacgacgctcttccgatct

SEQ ID NO: 16 - DNA, 24 bp, Artificial sequence
  Single strand DNA oligonucleotide
caagcagaagacggcatacgagat

SEQ ID NO: 17 - DNA, 6 bp, Artificial sequence
  Barcode sequence
tggtag

SEQ ID NO: 18 - DNA, 6 bp, Artificial sequence
  Barcode sequence
agcatg

SEQ ID NO: 19 - DNA, 6 bp, Artificial sequence
  Barcode sequence
atgtgc

SEQ ID NO: 20 - DNA, 6 bp, Artificial sequence
  Barcode sequence
cgagca

SEQ ID NO: 21 - DNA, 6 bp, Artificial sequence
  Barcode sequence
attgct

SEQ ID NO: 22 - DNA, 6 bp, Artificial sequence
  Barcode sequence
gcaact

SEQ ID NO: 23 - DNA, 6 bp, Artificial sequence
  Barcode sequence
aactgg

SEQ ID NO: 24 - DNA, 6 bp, Artificial sequence
  Barcode sequence
gctcaa

SEQ ID NO: 25 - DNA, 6 bp, Artificial sequence
  Barcode sequence
gttggt

SEQ ID NO: 26 - DNA, 6 bp, Artificial sequence
  Barcode sequence
tggacc

SEQ ID NO: 27 - DNA, 6 bp, Artificial sequence
  Barcode sequence
ttatac

SEQ ID NO: 28 - DNA, 6 bp, Artificial sequence
  Barcode sequence
agcgaa

SEQ ID NO: 29 - DNA, 6 bp, Artificial sequence
  Barcode sequence
caagtt

SEQ ID NO: 30 - DNA, 6 bp, Artificial sequence
  Barcode sequence
gatgtg

SEQ ID NO: 31 - DNA, 6 bp, Artificial sequence
  Barcode sequence
ttccga

SEQ ID NO: 32 - DNA, 6 bp, Artificial sequence
  Barcode sequence
atcctt

SEQ ID NO: 33 - DNA, 6 bp, Artificial sequence
  Barcode sequence
gtgtgc

SEQ ID NO: 34 - DNA, 6 bp, Artificial sequence
  Barcode sequence
gccaga

SEQ ID NO: 35 - DNA, 6 bp, Artificial sequence
  Barcode sequence
gctatg

SEQ ID NO: 36 - DNA, 6 bp, Artificial sequence
  Barcode sequence
ctcctg

SEQ ID NO: 37 - DNA, 6 bp, Artificial sequence
  Barcode sequence
ccgaca

SEQ ID NO: 38 - DNA, 6 bp, Artificial sequence
  Barcode sequence
cataat

SEQ ID NO: 39 - DNA, 6 bp, Artificial sequence
  Barcode sequence
ggtagg

SEQ ID NO: 40 - DNA, 6 bp, Artificial sequence
  Barcode sequence
taagta

SEQ ID NO: 41 - DNA, 6 bp, Artificial sequence
  Barcode sequence
ttcttc

SEQ ID NO: 42 - DNA, 6 bp, Artificial sequence
  Barcode sequence
gatcct

SEQ ID NO: 43 - DNA, 6 bp, Artificial sequence
  Barcode sequence
actgtc

SEQ ID NO: 44 - DNA, 6 bp, Artificial sequence
  Barcode sequence
catagg

SEQ ID NO: 45 - DNA, 6 bp, Artificial sequence
  Barcode sequence
aggcga

SEQ ID NO: 46 - DNA, 6 bp, Artificial sequence
  Barcode sequence
cgctat

SEQ ID NO: 47 - DNA, 6 bp, Artificial sequence
  Barcode sequence
ggcgac

SEQ ID NO: 48 - DNA, 6 bp, Artificial sequence
  Barcode sequence
tagaat

SEQ ID NO: 49 - DNA, 6 bp, Artificial sequence
  Barcode sequence
gtaacg

SEQ ID NO: 50 - DNA, 6 bp, Artificial sequence
  Barcode sequence
tcagac

SEQ ID NO: 51 - DNA, 6 bp, Artificial sequence
  Barcode sequence
gcgtaa

SEQ ID NO: 52 - DNA, 6 bp, Artificial sequence
  Barcode sequence
attcaa

SEQ ID NO: 53 - DNA, 6 bp, Artificial sequence
  Barcode sequence
tgtctt

SEQ ID NO: 54 - DNA, 6 bp, Artificial sequence
  Barcode sequence
ttgctg

SEQ ID NO: 55 - DNA, 6 bp, Artificial sequence
  Barcode sequence
gctgga

SEQ ID NO: 56 - DNA, 6 bp, Artificial sequence
  Barcode sequence
ctctgg

SEQ ID NO: 57 - DNA, 6 bp, Artificial sequence
  Barcode sequence
caagga

SEQ ID NO: 58 - DNA, 6 bp, Artificial sequence
  Barcode sequence
taacct

SEQ ID NO: 59 - DNA, 6 bp, Artificial sequence
  Barcode sequence
gatgac

SEQ ID NO: 60 - DNA, 6 bp, Artificial sequence
  Barcode sequence
cgaagg

SEQ ID NO: 61 - DNA, 6 bp, Artificial sequence
  Barcode sequence
ccgaga

SEQ ID NO: 62 - DNA, 6 bp, Artificial sequence
  Barcode sequence
gacaat

SEQ ID NO: 63 - DNA, 6 bp, Artificial sequence
  Barcode sequence
aggttc

SEQ ID NO: 64 - DNA, 6 bp, Artificial sequence
  Barcode sequence
tcatta

SEQ ID NO: 65 - DNA, 6 bp, Artificial sequence
  Barcode sequence
tgcgtt

SEQ ID NO: 66 - DNA, 6 bp, Artificial sequence
  Barcode sequence
gagttg

SEQ ID NO: 67 - DNA, 6 bp, Artificial sequence
  Barcode sequence
ttacag

SEQ ID NO: 68 - DNA, 6 bp, Artificial sequence
  Barcode sequence
tgctta

SEQ ID NO: 69 - DNA, 6 bp, Artificial sequence
  Barcode sequence
aacatt

SEQ ID NO: 70 - DNA, 6 bp, Artificial sequence
  Barcode sequence
ccgctg

SEQ ID NO: 71 - DNA, 6 bp, Artificial sequence
  Barcode sequence
ctggtc

SEQ ID NO: 72 - DNA, 6 bp, Artificial sequence
  Barcode sequence
tggata

SEQ ID NO: 73 - DNA, 6 bp, Artificial sequence
  Barcode sequence
acctgt

SEQ ID NO: 74 - DNA, 6 bp, Artificial sequence
  Barcode sequence
tgttgg

SEQ ID NO: 75 - DNA, 6 bp, Artificial sequence
  Barcode sequence
gacggc

SEQ ID NO: 76 - DNA, 6 bp, Artificial sequence
  Barcode sequence
cagata

SEQ ID NO: 77 - DNA, 6 bp, Artificial sequence
  Barcode sequence
cttagt

SEQ ID NO: 78 - DNA, 6 bp, Artificial sequence
  Barcode sequence
aaggcg

SEQ ID NO: 79 - DNA, 6 bp, Artificial sequence
  Barcode sequence
ctaggc

SEQ ID NO: 80 - DNA, 6 bp, Artificial sequence
  Barcode sequence
gcagca

SEQ ID NO: 81 - DNA, 6 bp, Artificial sequence
  Barcode sequence
ttacct

SEQ ID NO: 82 - DNA, 6 bp, Artificial sequence
  Barcode sequence
agttag

SEQ ID NO: 83 - DNA, 6 bp, Artificial sequence
  Barcode sequence
tgttac

SEQ ID NO: 84 - DNA, 6 bp, Artificial sequence
  Barcode sequence
attaca

SEQ ID NO: 85 - DNA, 6 bp, Artificial sequence
  Barcode sequence
gataat

SEQ ID NO: 86 - DNA, 6 bp, Artificial sequence
  Barcode sequence
gcatag

SEQ ID NO: 87 - DNA, 6 bp, Artificial sequence
  Barcode sequence
gtggac

SEQ ID NO: 88 - DNA, 6 bp, Artificial sequence
  Barcode sequence
agacaa

SEQ ID NO: 89 - DNA, 6 bp, Artificial sequence
  Barcode sequence
attgtt

SEQ ID NO: 90 - DNA, 6 bp, Artificial sequence
  Barcode sequence
aggaat

SEQ ID NO: 91 - DNA, 6 bp, Artificial sequence
  Barcode sequence
tccttc

SEQ ID NO: 92 - DNA, 6 bp, Artificial sequence
  Barcode sequence
tagcga

SEQ ID NO: 93 - DNA, 6 bp, Artificial sequence
  Barcode sequence
aactgt

SEQ ID NO: 94 - DNA, 6 bp, Artificial sequence
  Barcode sequence
ctattg

SEQ ID NO: 95 - DNA, 6 bp, Artificial sequence
  Barcode sequence
acggtc

SEQ ID NO: 96 - DNA, 6 bp, Artificial sequence
  Barcode sequence
tgcaga

SEQ ID NO: 97 - DNA, 6 bp, Artificial sequence
  Barcode sequence
tacagt

SEQ ID NO: 98 - DNA, 6 bp, Artificial sequence
  Barcode sequence
tgctgg

SEQ ID NO: 99 - DNA, 6 bp, Artificial sequence
  Barcode sequence
taggtc

SEQ ID NO: 100 - DNA, 6 bp, Artificial sequence
  Barcode sequence
cttgca

SEQ ID NO: 101 - DNA, 6 bp, Artificial sequence
  Barcode sequence
catgct

SEQ ID NO: 102 - DNA, 6 bp, Artificial sequence
  Barcode sequence
atagcg

SEQ ID NO: 103 - DNA, 6 bp, Artificial sequence
  Barcode sequence
gatatc

SEQ ID NO: 104 - DNA, 6 bp, Artificial sequence
  Barcode sequence
gttaca

SEQ ID NO: 105 - DNA, 6 bp, Artificial sequence
  Barcode sequence
cgacct

SEQ ID NO: 106 - DNA, 6 bp, Artificial sequence
  Barcode sequence
ccgcag

SEQ ID NO: 107 - DNA, 6 bp, Artificial sequence
  Barcode sequence
ggctgc

SEQ ID NO: 108 - DNA, 6 bp, Artificial sequence
  Barcode sequence
gattaa

SEQ ID NO: 109 - DNA, 6 bp, Artificial sequence
  Barcode sequence
gcacct

SEQ ID NO: 110 - DNA, 6 bp, Artificial sequence
  Barcode sequence
ccacag

SEQ ID NO: 111 - DNA, 25 bp, Artificial sequence
  P5 adapter sequence
aatgatacggcgaccaccgagatct

SEQ ID NO: 112 - DNA, 24 bp, Artificial sequence
  P7 adapter sequence
atctcgtatgccgtcttctgcttg

SEQ ID NO: 113 - DNA, 35 bp, Artificial sequence
  T7 RNA polymerase promoter sequence
cgattgaggccggtaatacgactcactataggggc

SEQ ID NO: 114 - DNA, 86 bp, Artificial Sequence
  Single strand DNA oligonucleotide
cgattgaggccggtaatacgactcactataggggcgacgtgtgctcttccgatctnnnnnnnnnnttttttttttttttttttttv

SEQ ID NO: 115 - DNA, 22 bp, Artificial Sequence
  Single strand DNA oligonucleotide
agatcggaagagcgtcgtgtag

SEQ ID NO: 116 - DNA, 22 bp, Artificial Sequence
  Single strand DNA oligonucleotide
tctagccttctcgcagcacatc

SEQ ID NO: 117 - DNA, 58 bp, Artificial Sequence; identical to SEQ ID NO: 15
  Single strand DNA oligonucleotide

SEQ ID NO: 118 - DNA, 58 bp, Artificial Sequence
  Single strand DNA oligonucleotide
caagcagaagacggcatacgagatgtgactggagttcagacgtgtgctcttccgatct